(19) United States
(12) Patent Application Publication
(10) Pub. No.: US 2003/0087246 A1
(43) Pub. Date: May 8, 2003

(54) Nucleic acids of the human ABCA12 gene, vectors containing such nucleic acids and uses thereof

(76) Inventors: Isabelle Arnould-Reguigne, Chennevieres Sur Marne (FR); Catherine Prades, Thiais (FR); Laurent Naudin, Etampes (FR); Cendrine Lemoine, Massy (FR); Michael Dean, Frederick, MD (US); Patrice Denefle, Saint Maur (FR); Marie-Francoise Rosier-Montus, Antony (FR)

Correspondence Address: Finnegan, Henderson, Farabow,, Garrett & Dunner, L.L.P., 1300 I Street, N.W., Washington, DC, 20005-3315, US

(21) Appl. No.: 10/072,900

(22) Filed: Feb. 12, 2002

Related U.S. Application Data
(60) Provisional application No. 60/267,715, filed on Feb. 12, 2001.

(51) Int. Cl. C12Q 1/68; C07H 21/04; C12P 19/34; C12P 21/02; C12N 5/06; C07K 14/435

(52) U.S. Cl. 435/6; 435/69.1; 435/320.1; 435/325; 530/350; 435/91.2; 536/23.5

(57) ABSTRACT

The present invention relates to a novel human ABCA12 gene as well as cDNAs encoding the novel full and short length ABCA12 proteins. The invention also relates to vectors and recombinants host cells comprising such nucleic acids, nucleotide probes and primers, and means for the detection of polymorphisms and mutations in the ABCA12 gene or in the corresponding protein produced by the allelic form of the ABCA12 gene.

The present invention relates to a novel ABCA gene, designated ABCA12, nucleic acids and cDNAs encoding novel ABCA12 proteins. The invention also relates to vectors and recombinant host cells, nucleotide probes and primers, as well as means for the detection of polymorphisms in general, and mutations in particular in the ABCA12 gene or corresponding proteins produced by the allelic forms of the ABCA12 gene.

The ABC (ATP-binding cassette transporter) gene superfamily encodes active transporter proteins and constitutes a family of proteins that are extremely well conserved during evolution, from bacteria to humans (Ames and Lecar, FASEB J., 1992, 6, 2660-2666). The ABC proteins are involved in extra- and intracellular membrane transport of various substrates, for example ions, metals amino acids, peptides, sugars, vitamins or steroid hormones. More precisely, some ABC transporters identified in mammals have function of chloride channel, multidrug resistance, bile salt transporter, glutathione conjugate transporter, HLA class I antigen transporter, sulfonylurea receptor, oligo A binding protein, or lipidic derivate (cholesterol, phosphatidylserine, . . . ) transporter. Among the 40 characterized humans members, 11 members have been described as associated with human disease, such as inter alia ABCA1, ABCA4 (ABCR) and ABCC7 (CFTR) which are thought to be involved in Tangier disease (Bodzioch M et al., Nat. Genet., 1999, 22(4); 347-351; Brooks-Wilson et al., Nat Genet, 1999, 22(4), 336-345; Rust S et al., Nat. Genet., 1999, 22, 352-355; Remaley A T et al., ), the Stargardt disease (Lewis R A et al., Am. J. Hum. Genet., 1999, 64, 422-434), and the Cystic Fibrosis (Riordan J M et al., Science, 1989, 245, 1066-1073), respectively. These implications reveal the importance of the functional role of the ABC gene family and the discovery of new family gene members should provide new insights into the physiopathology of human diseases.

The prototype ABC protein binds ATP and uses the energy from ATP hydrolysis to drive the transport of various molecules across cell membranes. The functional protein contains two ATP-binding domains (nucleotide binding fold, NBF) and two transmembrane (TM) domains. The genes are typically organized as full transporters containing two of each domain, or half transporters with only one of each domain. Most full transporters are arranged in a TM-NBF-TM-NBF fashion (Dean et al., Curr Opin Genet, 1995, 5, 79-785).

Analysis of amino acids sequence alignments of the ATP-binding domains has allowed the ABC genes to be separated into sub-families (Allikmets et al., Hum Mol Genet, 1996, 5, 1649-1655). Currently, according to the recent HUGO classification, seven ABC gene sub-families named ABC (A to G) have been described in the human genome (ABC1, CFTR/MRP, MDR, ABC8, ALD, GCN20, OABP) with all except one (OABP) containing multiple members. For the most part these sub-families contain genes that also display considerable conservation in the transmembrane domain sequences and have similar gene organization. However, ABC proteins transport very various substrates, and some members of different sub-families have been shown to share more similarity in substrate recognition than do proteins within same sub-family. Five of the sub-families are also represented in the yeast genome, indicating that these groups have been and retained early in the evolution of eukaryotes (Decottignies et al., Nat Genet, 1997, 137-45; Michaelis et al., 1995, Cold Spring Harbor Laboratory Press).

Several ABC transport proteins that have been identified in humans are associated with various diseases. For example, cystic fibrosis is caused by mutations in the ABCC7 gene or CFTR (cystic fibrosis transmembrane conductance regulator) gene (Riordan J M et al., Science, 1989, 245, 1066-1073). Also, mutations in the coding sequence of another gene belonging to the ABC gene sub-family “C”, the ABCC6 gene, have been recently identified as responsible of the phenotype of Pseudoxanthoma Elasticum (Le Saux et al., (2000), Nat.Genet 25(2), 223-7; Bergen et al. (2000) Nat Genet, 25(2):228-31). Pseudoxanthoma Elasticum is a genetic disorder of the connective tissue which is characterized by calcification of elastic fibers in skin, arteries and retina resulting in dermal and ocular lesions and arterial insufficiency. Likewise, a receptor for sulfonylureas, ABCC8 or SUR1, appears to be involved in type-I diabete insulin-dependent (IDDM). Moreover, some multiple drug resistance phenotypes in tumor cells have been associated with the gene encoding the MDR (multi-drug resistance) protein, which also has an ABC transporter structure (Anticancer Drug Des. April 1999;14(2):115-31). Other ABC transporters have been associated with neuronal and tumor conditions (U.S. Pat. No. 5,858,719) or potentially involved in diseases caused by impairment of the homeostasis of metals, such as ABC-3 protein. Likewise, another transport ABC, designated PFIC2, appears to be involved in a progressive familial intrahepatic choslestasia form, this protein being potentially responsible, in human, for the export of bile salts.

Among the ABC sub-families, the ABCA gene subfamily is probably the most evolutionary complex. The ABCA genes and OABP represent the only two sub-families of ABC genes that do not have identifiable orthologs in the yeast genome. There is, however, at least one ABCA-related gene in C. elegans (ced-7) and several in Drosophila. Thus the ABCA genes appear to have diverged after eukaryotes became multicellular and developed more sophisticated transport requirements. To date eleven members of the human ABCA sub-family have been described, making it the largest such group.

Full sequences of four genes of the ABCA sub-family have been described revealing a complex exon-intron structure. Best characterized ABCA genes are ABCA4, and ABCA1. In mammals the ABCA1 gene is highly expressed in macrophages and monocytes and is associated with the engulfinent of apoptotic cells (Luciani et al, Genomics (1994) 21, 150-9; Moynault et al., Biochem Soc Trans (1998) 26, 629-35; Wu et al., Cell (1998) 93, 951-60). The ced-7 gene, ortholog of ABCA1 in C. elegans, plays a role in the recognition and engulfinent of apoptotic cells suggesting a conserved function. Recently ABCA1 was demonstrated to be the gene responsible for Tangier disease, a disorder characterized by high levels of cholesterol in peripheral tissues, and a very low level of HDLs, and familial hypoalphalipoproteinemia (FHD) (Bodzioch et al., Nat Genet (1999) 22, 347-51; Brooks-Wilson et al., Nat Genet (1999) 336-45; Rust et al., Nat Genet (1999) 22, 352-5; Marcil et al., The Lancet (1999) 354, 1341-46). The ABCA1 protein is proposed to function in the reverse transport of cholesterol from peripheral tissues via an interaction with the apolipoprotein 1 (ApoA-l) of HDL tissues (Wang et al., JBC (2000). The ABCA2 gene is highly expressed in the brain and ABCA3 in the lung but no function has been ascribed to their respective chromosomal loci. The ABCA4 gene is exclusively expressed in the rod photoreceptors of the retina and mutations thereof are responsible for several pathologies of human eyes, such as retinal degenerative disorders retinoids (Allikmets et al., Science (1997) 277, 1805-1807; Allikmets et al., Nat Genet (1997) 15, 236-246; Sun et al., J Biol Chem (1999) 8269-81; Weng et al., Cell (1999) 98, 13-23; Cremers et al., Hum Mol Genet (1998) 7, 355-362; Martinez-Mir et al., Genomics (1997) 40, 142-146). ABCA4 is believed to transport retinal and/or retinal-phospholipid complexes from the rod photoreceptor outer segment disks to the cytoplasm, facilitating phototransduction.

Therefore, characterization of new genes from the ABCA subfamily is likely to yield biologically important transporters that may have an translocase activity for membrane lipid transport and may play a major role in human pathologies.

Lipids are water-insoluble organic biomolecules, which are essential components of diverse biological functions, including the storage, transport, and metabolism of energy, and membrane structure and fluidity. Lipids are derived from two sources in humans and other animals: some lipids are ingested as dietary fats and oils and other lipids are biosynthesized by the human or animal. In mammals at least 10% of the body weight is lipid, the bulk of which is in the form of triacylglycerols.

Triacylglycerols, also known as triglycerides and triacylglycerides, are made up of three fatty acids esterified to glycerol. Dietary triacylglycerols are stored in adipose tissues as a source of energy, or hydrolyzed in the digestive tract by triacylglycerol lipases, the most important of which is pancreatic lipase. Triacylglycerols are transported between tissues in the form of lipoproteins.

Lipoproteins are micelle-like assemblies found in plasma and contain varying proportions of different types of lipids and proteins (called apoproteins). There are five main classes of plasma lipoproteins, the major function of which is lipid transport. These classes are, in order of increasing density, chylomicrons, very low density lipoproteins (VLDL), intermediate-density lipoproteins (IDL), low density lipoproteins (LDL), and high density lipoproteins (HDL). Although many types of lipids are found associated with each lipoprotein class, each class transports predominantly one type of lipid: triacylglycerols are transported in chylomicrons, VLDL, and IDL; while phospholipids and cholesterol esters are transported in HDL and LDL respectively.

Phospholipids are di-fatty acid esters of glycerol phosphate, also containing a polar group coupled to the phosphate. Phospholipids are important structural components of cellular membranes. Phospholipids are hydrolyzed by enzymes called phospholipases. Phosphatidylcholine, an exemplary phospholipid, is a major component of most eukaryotic cell membranes.

Cholesterol is the metabolic precursor of steroid hormones and bile acids as well as an essential constituent of cell membranes. In humans and other animals, cholesterol is ingested in the diet and also synthesized by the liver and other tissues. Cholesterol is transported between tissues in the form of cholesteryl esters in LDLs and other lipoproteins.

Membranes surround every living cell, and serve as a barrier between the intracellular and extracellular compartments. Membranes also enclose the eukaryotic nucleus, make up the endoplasmic reticulum, and serve specialized functions such as in the myelin sheath that surrounds axons. A typical membrane contains about 40% lipid and 60% protein, but there is considerable variation. The major lipid components are phospholipids, specifically phosphatidylcholine and phosphatidylethanolamine, and cholesterol. The physicochemical properties of membranes, such as fluidity, can be changed by modification of either the fatty acid profiles of the phospholipids or the cholesterol content. Modulating the composition and organization of membrane lipids also modulates membrane-dependent cellular functions, such as receptor activity, endocytosis, and cholesterol flux.

High-density lipoproteins (HDL) are one of the five major classes of lipoproteins circulating in blood plasma. These lipoproteins are involved in various metabolic pathways such as lipid transport, the formation of bile acids, steroidogenesis, cell proliferation and, in addition, interfere with the plasma proteinase systems.

HDLs are perfect free cholesterol acceptors and, in combination with enzymatic activities such as that of the cholesterol ester transfer protein (CETP), the lipoprotein lipase (LPL), the hepatic lipase (HL) and the lecithin:cholesterol acyltransferase (LCAT), play a major role in the reverse transport of cholesterol, that is to say the transport of excess cholesterol in the peripheral cells to the liver for its elimination from the body in the form of bile acid. It has been demonstrated that the HDLs play a central role in the transport of cholesterol from the peripheral tissues to the liver.

Various diseases linked to an HDL deficiency have been described, including Tangier, FHD disease, and LCAT deficiency. In addition, HDL-cholesterol deficiencies have been observed in patients suffering from malaria and diabetes (Nilsson et al., 1990, J. Intern. Med., 227:151-5; Djoumessi, 1989, Pathol Biol., 37 :909-11; Mohanty et al., 1992. Ann Trop Med Parasitol., 86 :601-6; Maurois et al., 1985, Biochimie, 67 :227-39; Grellier et al., 1997, Vox Sang, 72 :211-20; Agbedana et al., 1990, Ann Trop Med Parasitol., 84 :529-30; Cuisinier et al., 1990, Med Trop, 50 :91-5; Davis et al., 1993, J. Infect. 26 :279-85; Davis et al., 1995, J. Infect. 31:181-8; Pirich et al., 1993, Semin Thromb Hemost., 19:138-43; Tomlinson and Raper, 1996, Nat. Biotechnol., 14:717-21; Hager and Hajduk, 1997, Nature 385:823-6; Kwiterovich, 1995, Ann NY Acad Sci., 748 :313-30; Syvanne et al. 1995, Circulation, 92:364-70; and Syvanne et al., 1995, J.Lipid Res., 36:573-82). The deficiency involved in Tangier and/or FHD disease is linked to a cellular defect in the translocation of cellular cholesterol which causes a degradation of the HDLs and leads to a disruption in the lipoprotein metabolism.

Atherosclerosis is defined in histological terms by deposits (lipid or fibrolipid plaques) of lipids and of other blood derivatives in blood vessel walls, especially the large arteries (aorta, coronary arteries, carotid). These plaques, which are more or less calcified according to the degree of progression of the atherosclerosis process, may be coupled with lesions and are associated with the accumulation in the vessels of fatty deposits consisting essentially of cholesterol esters. These plaques are accompanied by a thickening of the vessel wall, hypertrophy of the smooth muscle, appearance of foam cells (lipid-laden cells resulting from uncontrolled uptake of cholesterol by recruited macrophages) and accumulation of fibrous tissue. The atheromatous plaque protrudes markedly from the wall, endowing it with a stenosing character responsible for vascular occlusions by atheroma, thrombosis or embolism, which occur in those patients who are most affected. These lesions can lead to serious cardiovascular pathologies such as myocardial infarction, sudden death, cardiac insufficiency, and stroke.

Mutations within genes that play a role in lipoprotein metabolism have been identified. Specifically, several mutations in the apolipoprotein apoA-I gene have been characterized. These mutations are rare and may lead to a lack of production of apoA-I. Mutations in the genes encoding LPL or its activator apoC-II are associated with severe hypertriglyceridemias and substantially reduced HDL-C levels. Mutations in the gene encoding the enzyme LCAT are also associated with a severe HDL deficiency.

In addition, dysfunctions in the reverse transport of cholesterol may be induced by physiological deficiencies affecting one or more of the steps in the transport of stored cholesterol, from the intracellular vesicles to the membrane surface where it is accepted by the HDLs.

Diabete is defined as a disorder of carbohydrate metabolism caused by absence or deficiency of insulin, insulin resistance, or both, ultimately leading to hyperglycemia. Diabete mellitus is typically classified into two main subtypes: type-I or insulin-dependent diabetes (IDDM), and type-II or non-insulin-dependent diabetes (NIDDM). A more accurate way to differentiate the two would be to classify the insulin dependent diabetic as ketoacidosis-prone, and the non-insulin-dependent diabetic as ketoacidosis-resistant. Type-I and II would be differentiated on immunological-etiological grounds with type-I referring to an immune-mediated condition, whereas type-II is non-immune-mediated (Foster et al., Diabetes Mellitus. In: Braunwald E, Isselbacher K J, Petersdorf R G, et al, eds. Harrison's Principles of Internal Medicine. 11th ed. New York: McGraw-Hill; 1988:1778-1781). Diabetes mellitus markedly increases the risk of death and disability from the various complications of atherosclerosis. In effect, about 80% of adult diabetic patients die from coronary heart disease (CHD), cerebrovascular disease, and/or peripheral vascular disease. Elevated LDL cholesterol, reduced HDL cholesterol, and hypertriglyceridemia are frequently found in insulin-dependent diabetes mellitus (IDDM) and non-insulin-dependent diabetes mellitus (NIDDM). There is considerable evidence that higher blood triglycerides and lower HDL cholesterol may be intrinsically related to the abnormal physiology produced by insulin resistance or inadequate insulin action, with the concomitant metabolic disturbances. It is believed that type-I diabetes has a genetic component which must be present for susceptibility to occur, and such an IDDM susceptiblity gene has been mapped to chromosome 2q34.

Lamellar Ichthyosis is an inherited autosomal recessive disorder of cornification. It can be life-threatening soon after bearth, since the neonate skin is covered by a thick collodion-like membrane, exposing the infant to sepsis and dramatic dehydration. It is also variously accompanied by palmoplantar keratoderma, alopecia and erythema. Type 1 lamellar ichthyosis maps to chromosome 14q11 and it was recently demonstrated to result from deleterious mutations in the transglutaminase 1 (TGM1) gene (Parmentier et al., Hum Mol Genet (1995) 4: 1391-1395; Huber et al., Science (1995) 267: 525-538; Russell et al., Nat Genet (1995) 9: 279-283; Laiho et al., Am J Hum Genet (1997) 61: 529-538; Huber et al., J Biol Chem (1997) 272: 21018-21026; Petit et al., Eur J Hum Genet (1997) 5: 218-228). This gene directs the construction of the cornified envelope, a protein structure underneath the plasma membrane of keratinocytes which forms during their late-stage terminal differentiation. Another form, designated type 2 lamellar ichthyosis, was mapped to chromosome 2q33-q35 (ICR2B locus; Parmentier et al., Hum Molec Genet (1996) 5: 555-559) but the causative gene is yet unknown. This region has been narrowed to a roughly 2 Mb region flanked by D2S143 and D2S137 markers (Parmentier et al., Eur J Hum Genet (1999) 7: 77-87).

Cataract is one of the major causes of blindness in humans. Genetic linkage analysis performed with families with polymorphic congenital cataract evidenced linkage for chromosome 2q33-35, more precisely near D2S72 and TNP1 (Rogaev et al., Hum Mol Genet (1996) 5: 699-703). Many forms of hereditary congenital human cataracts have been described as isolated abnormalities. The opacities of the lens leading to broad variability in cataracts may be caused by different mechanisms. Therefore, crystallin genes or genes encoding enzymes modifying the crystallin proteins are candidates. Crystallin genes and pseudogenes have been mapped to various regions of the genome, among which 2q33-q36 region for the gamma-crystallins (Shiloh et al., Hum Genet (1986) 73: 17-19).

The applicant have discovered and characterized a new gene belonging to the ABC transporter superfamily and more precisely belonging to the ABCA protein sub-family, and it has been designated ABCA12. Different transcripts isoforms have been identified since the ABCA12 gene has two different polyadenylation sites, and two splicing forms. Consequently, four different mRNA ABCA12 were found to be expressed in humans. The two messengers which result of alternative splicing encode two putative ABCA12 proteins having different lengths, a full length ABCA12 protein as well as a shorter isoform. Both the full length ABCA12 proteins show considerable conservation of the amino acid sequences, particularly within the transmembrane region (TM) and the ATP-binding regions 1 and 2 (NBD1 and NBD2), and have a similar gene organization.

Further, we have mapped the novel ABCA12 gene in a region located in the 2q34 locus of human chromosome 2, which is statistically linked with pathologies such as lamellar Ichthyosis (Parmentier et al., Europ J Hum Genet (1999) 7:77-87; Parmentier et al, Hum Mol Genet (1996) 5(4) 555-9), polymorphic congenital cataract, and insulin dependant diabete mellitus (IDDM13) (Morahan et al., Science (1996) 272 (5269) 1811-1813). This result supports the hypothesis that ABCA12 is a positional candidate for these three disorders that the novel ABCA12 gene may be one causing gene for the phenotype of these pathologies.

Furthermore, an electronic analysis of tissue distribution has been performed, and sequence of the ABCA12 transcript has been shown to match with various ESTs generated by skin/epithelial cell cDNA library sequencing, suggesting a preferential tissue expression in the skin/epithelium. This reinforces the hypothesis of involvement of ABCA12 in Ichthyosis for instance as this is factually a positional and regional candidate, based on genome mapping and tissue distribution data.

### SUMMARY OF THE INVENTION

The present invention relates to nucleic acids corresponding to the human ABCA12 gene, cDNAs and protein isoforms, which are likely to be involved in the transport of various substrates comprising sugars, metals, aminoacids, or vitamins. More precisely, they function in mammals as chloride channel, multidrug resistance, bile salt transporter, glutathione conjugate transporter, HLA class I antigen transporter, sulfonylurea receptor, or lipidic derivate transporter, in particular substances such as cholesterol, phosphaditylserine, or in any pathology whose candidate chromosomal region is situated on chromosome 2, more precisely on the 2q arm and still more precisely in the 2q34 locus.

Thus, a first subject of the invention is a nucleic acid comprising a nucleotide sequence of any one of SEQ ID NOs: 1-4, or a complementary nucleotide sequence thereof.

The invention also relates to a nucleic acid comprising at least 8 consecutive nucleotides of a nucleotide sequence of any one of SEQ ID NOs: 1-4 or a complementary nucleotide sequence thereof.

The invention also relates to a nucleic acid having at least 80% nucleotide identity with a nucleic acid comprising a nucleotide sequence of any one of SEQ ID NOs: 1-4, or a complementary nucleotide sequence thereof.

The invention also relates to a nucleic acid having at least 85%, preferably 90%, more preferably 95% and still more preferably 98% nucleotide identity with a nucleic acid comprising a nucleotide sequence of any one of SEQ ID NOs: 1-4, or a complementary nucleotide sequence thereof.

The invention also relates to a nucleic acid hybridizing, under high stringency conditions, with a nucleotide sequence of any one of SEQ ID NOs: 1-4, or a complementary nucleotide sequence thereof.

The invention also relates to nucleic acids, particularly cDNA molecules, which encode the full length human ABCA12 proteins isoforms. Thus, the invention relates to a nucleic acid comprising a nucleotide sequence of any one of SEQ ID NO: 1-4, or a complementary nucleotide sequence.

The invention also relates to a nucleic acid comprising a nucleotide sequence as depicted in SEQ ID NO: 1-4, or a complementary nucleotide sequence.

According to the invention, a nucleic acid comprising a nucleotide sequence of SEQ ID NO: 1 or 3, which encodes a full length ABCA12 polypeptide of 2595 amino acids comprising the amino acid sequence of SEQ ID NO: 5.

According to the invention, a nucleic acid comprising a nucleotide sequence of SEQ ID NO: 2 or 4, which encodes a full length ABCA12 polypeptide of 2516 amino acids comprising the amino acid sequence of SEQ ID NO: 6.

Thus, the invention also relates to a nucleic acid encoding a polypeptide comprising an amino acid sequence of any one of SEQ ID NO: 5 or 6.

Thus, the invention also relates to a polypeptide comprising an amino acid sequence of any one of SEQ ID NO: 5 or 6.

The invention also relates to a polypeptide comprising an amino acid sequence as depicted in any one of SEQ ID NO: 5 or 6.

The invention further relates to a means for detecting polymorphisms in general, and mutations in particular, in the ABCA12 gene or corresponding proteins produced by the allelic forms of this gene.

According to another aspect, the invention also relates to the nucleotide sequences of ABCA12 gene comprising at least one biallelic polymorphism such as for example a substitution, addition or deletion of one or more nucleotides.

Nucleotide probes and primers hybridizing with a nucleic acid sequence located in the region of the ABCA12 nucleic acid (genomic DNA, messenger RNA, cDNA), in particular, a nucleic acid sequence comprising any one of the mutations or polymorphisms.

The nucleotide probes or primers according to the invention comprise at least 8 consecutive nucleotides of a nucleic acid comprising any one of SEQ ID NOs: 1-4 or a complementary nucleotide sequence thereof.

Preferably, nucleotide probes or primers according to the invention will have a length of 10, 12, 15, 18 or 20 to 25, 35, 40, 50, 70, 80, 100, 200, 500, 1000, 1500 consecutive nucleotides of a nucleic acid according to the invention, in particular of a nucleic acid comprising any one of SEQ ID NOs: 1-4, or a complementary nucleotide sequence thereof.

Alternatively, a nucleotide probe or primer according to the invention will consist of and/or comprise fragments having a length of 12, 15, 18, 20, 25, 35, 40, 50, 100, 200, 500, 1000, 1500 consecutive nucleotides of a nucleic acid according to the invention, more particularly of a nucleic acid comprising any one of SEQ ID NOs: 1-4, or a complementary nucleotide sequence thereof.

The definition of a nucleotide probe or primer according to the invention therefore covers oligonucleotides which hybridize, under the high stringency hybridization conditions defined above, with a nucleic acid comprising any one of SEQ ID NOs: 1-4, or a complementary nucleotide sequence thereof.

The preferred probes and primers according to the invention comprise all or part of a nucleotide sequence comprising any one of SEQ ID NOs: 7-38, or a complementary nucleotide sequence thereof.

The nucleotide primers according to the invention may be used to amplify any one of the nucleic acids according to the invention, and more particularly a nucleic acid comprising a nucleotide sequence of any one of SEQ ID NOs: 1-4, or a complementary nucleotide sequence thereof.

According to the invention, some nucleotide primers specific for an ABCA12 gene, may be used to amplify a nucleic acid comprising a SEQ ID NOs: 1-4, and comprise a nucleotide sequence of any one of SEQ ID NOs:7-38, or a complementary nucleotide sequence thereof.

Another subject of the invention relates to a method of amplifying a nucleic acid according to the invention, and more particularly a nucleic acid comprising a) any one of SEQ ID NOs: 1-4, a complementary nucleotide sequence thereof, or b) as depicted in any one of SEQ ID NOs: 1-4, or a complementary nucleotide sequence thereof, contained in a sample, said method comprising the steps of:

a) bringing the sample in which the presence of the target nucleic acid is suspected into contact with a pair of nucleotide primers whose hybridization position is located respectively on the 5′ side and on the 3′ side of the region of the target nucleic acid whose amplification is sought, in the presence of the reagents necessary for the amplification reaction; and

b) detecting the amplified nucleic acids.

The present invention also relates to a method of detecting the presence of a nucleic acid comprising a nucleotide sequence of any one of SEQ ID NOs: 1-4, or a complementary nucleotide sequence, or a nucleic acid fragment or variant of any one of SEQ ID NOs: 1-4, or a complementary nucleotide sequence in a sample, said method comprising the steps of:

1) bringing one or more nucleotide probes according to the invention into contact with the sample to be tested;

2) detecting the complex which may have formed between the probe(s) and the nucleic acid present in the sample.

According to a specific embodiment of the method of detection according to the invention, the oligonucleotide probes are immobilized on a support.

According to another aspect, the oligonucleotide probes comprise a detectable marker.

Another subject of the invention is a box or kit for amplifying all or part of a nucleic acid comprising a) any one of SEQ ID NOs: 1-4, or a complementary nucleotide sequence thereof, or b) as depicted in any one of SEQ ID NOs: 1-4 or of a complementary nucleotide sequence thereof, said box or kit comprising:

1) a pair of nucleotide primers in accordance with the invention, whose hybridization position is located respectively on the 5′ side and 3′ side of the target nucleic acid whose amplification is sought; and optionally,

2) reagents necessary for an amplification reaction.

Such an amplification box or kit will preferably comprise at least one pair of nucleotide primers as described above.

The invention also relates to a box or kit for detecting the presence of a nucleic acid according to the invention in a sample, said box or kit comprising:

a) one or more nucleotide probes according to the invention;

b) appropriate reagents necessary for a hybridisation reaction.

According to a first aspect, the detection box or kit is characterized in that the nucleotide probe(s) and primer(s)are immobilized on a support.

According to a second aspect, the detection box or kit is characterized in that the nucleotide probe(s) and primer(s) comprise a detectable marker.

According to a specific embodiment of the detection kit described above, such a kit will comprise a plurality of oligonucleotide probes and/or primers in accordance with the invention which may be used to detect target nucleic acids of interest or alternatively to detect mutations in the coding regions or the non-coding regions of the nucleic acids according to the invention. According to preferred embodiment of the invention, the target nucleic acid comprises a nucleotide sequence of any one of SEQ ID NOs: 1-4, or of a complementary nucleic acid sequence. Alternatively, the target nucleic acid is a nucleic acid fragment or variant of a nucleic acid comprising any one of SEQ ID NOs: 1-4, or of a complementary nucleotide sequence.

According to another preferred embodiment, a primer according to the invention comprises, generally, all or part of any one of SEQ ID NOs: 1-4, or a complementary sequence.

The invention also relates to a recombinant vector comprising a nucleic acid according to the invention. Preferably, such a recombinant vector will comprise a nucleic acid selected from the group consisting of

a) a nucleic acid comprising a nucleotide sequence of any one of SEQ ID NOs: 1-4, or a complementary nucleotide sequence thereof,

b) a nucleic acid comprising a nucleotide sequence as depicted in any one of SEQ ID NOs: 1-4, or a complementary nucleotide sequence thereof,

c) a nucleic acid having at least eight consecutive nucleotides of a nucleic acid comprising a nucleotide sequence of any one of SEQ ID NOs: 1-4, or a complementary nucleotide sequence thereof;

d) a nucleic acid having at least 80% nucleotide identity with a nucleic acid comprising a nucleotide sequence of any one of SEQ ID NOs: 1-4, or a complementary nucleotide sequence thereof;

e) a nucleic acid having 85%, 90%, 95%, or 98% nucleotide identity with a nucleic acid comprising a nucleotide sequence of any one of SEQ ID NOs: 1-4, or a complementary nucleotide sequence thereof;

f) a nucleic acid hybridizing, under high stringency hybridization conditions, with a nucleic acid comprising a nucleotide sequence of any one of SEQ ID NOs: 1-4, or a complementary nucleotide sequence; and

g) a nucleic acid encoding a polypeptide comprising an amino acid sequence of SEQ ID NO: 5-6.

According to a first embodiment, a recombinant vector according to the invention is used to amplify a nucleic acid inserted therein, following transformation or transfection of a desired cellular host.

According to a second embodiment, a recombinant vector according to the invention corresponds to an expression vector comprising, in addition to a nucleic acid in accordance with the invention, a regulatory signal or nucleotide sequence that directs or controls transcription and/or translation of the nucleic acid and its encoded mRNA.

According to a preferred embodiment, a recombinant vector according to the invention will comprise in particular the following components:

(1) an element or signal for regulating the expression of the nucleic acid to be inserted, such as a promoter and/or enhancer sequence;

(2) a nucleotide coding region comprised within the nucleic acid in accordance with the invention to be inserted into such a vector, said coding region being placed in phase with the regulatory element or signal described in (1); and

(3) an appropriate nucleic acid for initiation and termination of transcription of the nucleotide coding region of the nucleic acid described in (2).

The present invention also relates to a defective recombinant virus comprising a cDNA nucleic acid encoding any one of short or full length ABCA12 polypeptide involved in the transport of lipophilic substances, or in any pathology whose candidate chromosomal region is located on chromosome 2, more precisely on the 2q arm and still more precisely in the 2q34 locus.

In another preferred embodiment of the invention, the defective recombinant virus comprises a gDNA nucleic acid encoding any one of ABCA12 polypeptides isoforms involved in the transport of lipophilic substances. Preferably, the ABCA12 polypeptides isoforms comprise amino acid sequences selected from SEQ ID NO: 5-6, respectively.

In another preferred embodiment, the invention relates to a defective recombinant virus comprising a nucleic acid encoding the full length or short ABCA12 polypeptide under the control of a promoter chosen from RSV-LTR or the CMV early promoter.

According to a specific embodiment, a method of introducing a nucleic acid according to the invention into a host cell, in particular a host cell obtained from a mammal, in vivo, comprises a step during which a preparation comprising a pharmaceutically compatible vector and a “naked” nucleic acid according to the invention, placed under the control of appropriate regulatory sequences, is introduced by local injection at the level of the chosen tissue, for example a smooth muscle tissue, the “naked” nucleic acid being absorbed by the cells of this tissue.

According to a specific embodiment of the invention, a composition is provided for the in vivo production of any one of the ABCA12 proteins isoforms. This composition comprises a nucleic acid encoding the ABCA12 polypeptides placed under the control of appropriate regulatory sequences, in solution in a physiologically acceptable vehicle and/or excipient.

Therefore, the present invention also relates to a composition comprising a nucleic acid encoding the short or full length ABCA12 polypeptide comprising an amino acid sequence selected from SEQ ID NO: 5 or 6, wherein the nucleic acid is placed under the control of appropriate regulatory elements.

Consequently, the invention also relates to a pharmaceutical composition intended for the prevention of or treatment of a patient or subject affected by a lamellar ichthyosis comprising a nucleic acid encoding any one of the short or full lenth ABCA12 protein, in combination with one or more physiologically compatible excipients.

The invention further relates to a pharmaceutical composition intended for the prevention of or treatment of a patient or subject affected by an insulin dependant diabete mellitus (IDDM13) comprising a nucleic acid encoding the short or full length ABCA12 protein, in combination with one or more physiologically compatible excipients.

The invention further relates to a pharmaceutical composition intended for the prevention of or treatment of a patient or subject affected by a polymorphic congenital cataract comprising a nucleic acid encoding the short or full length ABCA12 protein, in combination with one or more physiologically compatible excipients.

Preferably, such a composition will comprise a nucleic acid comprising a nucleotide sequence of any one of SEQ ID NO:1-4, wherein the nucleic acid is placed under the control of an appropriate regulatory element or signal.

In addition, the present invention is directed to a pharmaceutical composition intended for the prevention of or treatment of a patient or a subject affected by a pathology located on the chromosome locus 2q34, such as IDDM, the ichthyosis lamellar, the polymorphic congenital cataract, comprising a recombinant vector according to the invention, in combination with one or more physiologically compatible excipients.

The invention also relates to the use of a nucleic acid according to the invention encoding the short or full length ABCA12 protein for the manufacture of a medicament intended for the prevention or treatment of subject affected by a dysfunction of transport of lipophilic substances.

The invention also relates to the use of a recombinant vector according to the invention comprising a nucleic acid encoding any one of ABCA12 proteins isoforms for the manufacture of a medicament intended for the prevention or the treatment of a subject affected by a dysfunction of transport of lipophilic substances, or by a pathology located on the chromosome locus 2q34 such as for example the lamellar ichthyosis, the polymorphic congenital cataract, or insulin-dependant diabete mellitus.

The subject of the invention is therefore also a recombinant vector comprising a nucleic acid according to the invention that encodes any one of ABCA12 proteins or polypeptides isoforms involved in the transport of liphophilic substances, or in a pathology located on the chromosome locus 2q34 such as for example the lamellar ichthyosis, the polymorphic congenital cataract, or insulin-dependant diabete mellitus.

The invention also relates to the use of such a recombinant vector for the preparation of a pharmaceutical composition intended for the treatment and/or for the prevention of diseases or conditions associated with deficiency of lipophilic substances or with a pathology located on the chromosome locus 2q34 such as for example the lamellar ichthyosis, the polymorphic congenital cataract, or insulin-dependant diabete mellitus.

The present invention also relates to the use of cells genetically modified ex vivo with such a recombinant vector according to the invention, or cells producing a recombinant vector, wherein the cells are implanted in the body, to allow a prolonged and effective expression in vivo of any one biologically active ABCA12 polypeptides isoforms.

The invention also relates to the use of a nucleic acid according to the invention encoding any one of ABCA12 protein isoforms for the manufacture of a medicament intended for the prevention and/or the treatment of subjects affected by a dysfunction of lipophilic substances transport or by a pathology located on the chromosome locus 2q34 such as for example the lamellar ichthyosis, the polymorphic congenital cataract, or insulin-dependant diabete mellitus.

The invention also relates to the use of a recombinant vector according to the invention comprising a nucleic acid encoding any one of ABCA12 polypeptide isoforms according to the invention for the manufacture of a medicament intended for the prevention and/or the treatment of subjects affected by a dysfunction of lipophilic substances transport or by a pathology located on the chromosome locus 2q34 such as for example the lamellar ichthyosis, the polymorphic congenital cataract, or insulin-dependant diabete mellitus.

The invention also relates to the use of a recombinant host cell according to the invention, comprising a nucleic acid encoding any one of ABCA12 polypeptide isoforms according to the invention for the manufacture of a medicament intended for the prevention and/or the treatment of subjects affected by a dysfunction of lipophilic transport or by a pathology located on the chromosome locus 2q34 such as for example the lamellar ichthyosis, the polymorphic congenital cataract, or insulin-dependant diabete mellitus.

The present invention also relates to the use of a recombinant vector according to the invention, preferably a defective recombinant virus, for the preparation of a pharmaceutical composition for the treatment and/or prevention of pathologies linked to the dysfunction of lipophilic substances transport or located on the chromosome locus 2q34 such as for example the lamellar ichthyosis, the polymorphic congenital cataract, or insulin-dependant diabete mellitus.

The invention relates to the use of such a recombinant vector or defective recombinant virus for the preparation of a pharmaceutical composition intended for the treatment and/or for the prevention of cardiovascular disease linked to a deficiency in the transport of lipophilic substances or of a pathology located on the chromosome locus 2q34 such as for example the lamellar ichthyosis, the polymorphic congenital cataract, or insulin-dependant diabete mellitus. Thus, the present invention also relates to a pharmaceutical composition comprising one or more recombinant vectors or defective recombinant viruses according to the invention.

The present invention also relates to the use of cells genetically modified ex vivo with a virus according to the invention, or of cells producing such viruses, implanted in the body, allowing a prolonged and effective expression in vivo of any one biologically active of ABCA12 proteins.

The present invention shows that it is possible to incorporate a nucleic acid encoding an ABCA12 polypeptide isoform according to the invention into a viral vector, and that these vectors make it possible to effectively express a biologically active, mature polypeptide. More particularly, the invention shows that the in vivo expression of one isoform of ABCA12 proteins may be obtained by direct administration of an adenovirus or by implantation of a producing cell or of a cell genetically modified by an adenovirus or by a retrovirus incorporating such a nucleic acid.

In this regard, another subject of the invention relates to any mammalian cell infected with one or more defective recombinant viruses according to the invention. More particularly, the invention relates to any population of human cells infected with these viruses. These may be in particular cells of blood origin (totipotent stem cells or precursors), fibroblasts, myoblasts, hepatocytes, keratinocytes, smooth muscle and endothelial cells, glial cells and the like.

Another subject of the invention relates to an implant comprising mammalian cells infected with one or more defective recombinant viruses according to the invention or cells producing recombinant viruses, and an extracellular matrix. Preferably, the implants according to the invention comprise 10⁵ to 10¹⁰ cells. More preferably, they comprise 10⁶ to 10⁸ cells.

More particularly, in the implants of the invention, the extracellular matrix comprises a gelling compound and optionally, a support allowing the anchorage of the cells.

The invention also relates to a recombinant host cell comprising a nucleic acid of the invention, and more particularly, a nucleic acid comprising any one of SEQ ID NO: 1-4, or a complementary nucleotide sequence thereof.

The invention also relates to a recombinant host cell comprising a nucleic acid of the invention, and more particularly a nucleic acid comprising a nucleotide sequence as depicted in any one SEQ ID NO: 1-4, or a complementary nucleotide sequence thereof.

According to another aspect, the invention also relates to a recombinant host cell comprising a recombinant vector according to the invention. Therefore, the invention also relates to a recombinant host cell comprising a recombinant vector comprising any of the nucleic acids of the invention, and more particularly a nucleic acid comprising any one nucleotide sequence of SEQ ID NO: 1-4, or a complementary nucleotide sequence thereof.

Specifically, the invention relates to a recombinant host cell comprising a recombinant vector comprising a nucleic acid comprising any one of SEQ ID NOs: 1-4, or a complementary nucleotide sequence thereof.

The invention also relates to a recombinant host cell comprising a recombinant vector comprising a nucleic acid comprising a nucleotide sequence as depicted in any one of SEQ ID NOs: 1-4, or of a complementary nucleotide sequence thereof.

The invention also relates to a recombinant host cell comprising a recombinant vector comprising a nucleic acid encoding a polypeptide comprising any one amino acid sequence of SEQ ID NO:5 or 6.

The invention also relates to a method for the production of a polypeptide comprising an amino acid sequence of any one of SEQ ID NOs: 5 or 6, or of a peptide fragment or a variant thereof, said method comprising the steps of:

a) inserting a nucleic acid encoding said polypeptide into an appropriate vector;

b) culturing, in an appropriate culture medium, a previously transformed host cell or transfecting a host cell with the recombinant vector of step a);

c) recovering the conditioned culture medium or lysing the host cell, for example by sonication or by osmotic shock;

d) separating and purifying said polypeptide from said culture medium or alternatively from the cell lysates obtained in step c); and

e) where appropriate, characterizing the recombinant polypeptide produced.

A polypeptide termed “homologous” to a polypeptide having an amino acid sequence selected from SEQ ID NO: 5 or 6 also forms part of the invention. Such a homologous polypeptide comprises an amino acid sequence possessing one or more substitutions of an amino acid by an equivalent amino acid.

The ABCA12 polypeptides isoforms according to the invention, in particular 1) a polypeptide comprising an amino acid sequence of any one of SEQ ID NOs: 5 or 6, 2) a polypeptide fragment or variant of a polypeptide comprising an amino acid sequence of any one of SEQ ID NOs: 5 or 6, or 3) a polypeptide termed “homologous” to a polypeptide comprising amino acid sequence selected from SEQ ID NO: 5 or 6.

In a specific embodiment, an antibody according to the invention is directed against 1) a polypeptide comprising an amino acid sequence of any one of SEQ ID NOs: 5 or 6, 2) a polypeptide fragment or variant of a polypeptide comprising an amino acid sequence selected from SEQ ID NOs: 5 or 6, or 3) a polypeptide termed “homologous” to a polypeptide comprising amino acid sequence selected from SEQ ID NO: 5 or 6. Such antibody is produced by using the trioma technique or the hybridoma technique described by Kozbor et al. (Immunology Today, (1983) 4:72).

Thus, the subject of the invention is, in addition, a method of detecting the presence of any one of the polypeptides according to the invention in a sample, said method comprising the steps of:

a) bringing the sample to be tested into contact with an antibody directed against 1) a polypeptide comprising an amino acid sequence of any one of SEQ ID NOs: 5 or 6, 2) a polypeptide fragment or variant of a polypeptide comprising an amino acid sequence selected from SEQ ID NOs: 5 or 6, 3) a polypeptide termed “homologous” to a polypeptide comprising amino acid sequence of any one of SEQ ID NO: 5 or 6, and

b) detecting the antigen/antibody complex formed.

The invention also relates to a box or kit for diagnosis or for detecting the presence of any one of polypeptide in accordance with the invention in a sample, said box comprising:

a) an antibody directed against 1) a peptide having an amino acid sequence of any one of SEQ ID NOs: 5 or 6, 2) a polypeptide fragment or variant of a polypeptide comprising an amino acid sequence of any one of SEQ ID NOs: 5 or 6, or 3) a polypeptide “homologous” to a polypeptide comprising amino acid sequence of SEQ ID NO: 5 or 6, and

b) a reagent allowing the detection of the antigen/antibody complexes formed.

The invention also relates to a pharmaceutical composition comprising a nucleic acid according to the invention.

The invention also provides pharmaceutical compositions comprising a nucleic acid encoding any one of ABCA12 polypeptide isoforms according to the invention and pharmaceutical compositions comprising any one of ABCA12 polypeptides according to the invention intended for the prevention or treatment of diseases linked to a deficiency of lipophilic substances transport or a pathology located on the chromosome locus 2q34 such as for example the lamellar ichthyosis, the polymorphic congenital cataract, or insulin-dependant diabete mellitus.

The present invention also relates to a new therapeutic approach for the treatment of pathologies linked to deficiency of the ABC A12 gene or lipophilic substances transport, comprising transferring and expressing in vivo nucleic acids encoding any one of ABCA12 protein isoforms according to the invention.

Thus, the present invention offers a new approach for the treatment and/or prevention of pathologies linked to deficiencies of the ABC A12 gene or abnormalities of transport of lipophilic substances or any pathology located on the chromosome locus 2q34 such as for example the lamellar ichthyosis, the polymorphic congenital cataract, or insulin-dependant diabete mellitus. Specifically, the present invention provides methods to restore or promote improved lipophilic substances transport in a patient or subject.

Consequently, the invention also relates to a pharmaceutical composition intended for the prevention and/or treatment of subjects affected by a dysfunction of lipophilic substances transport, comprising a nucleic acid encoding any one of the ABCA12 proteins isoforms, in combination with one or more physiologically compatible vehicle and/or excipient.

According to a specific embodiment of the invention, a composition is provided for the in vivo production of any one of the ABCA12 proteins. This composition comprises a nucleic acid encoding any one of the ABCA12 polypeptides placed under the control of appropriate regulatory sequences, in solution in a physiologically compatible vehicle and/or excipient.

Therefore, the present invention also relates to a composition comprising a nucleic acid encoding a polypeptide comprising an amino acid sequence of any one of ID NO: 5 or 6, wherein the nucleic acid is placed under the control of appropriate regulatory elements.

Preferably, such a composition will comprise a nucleic acid comprising a nucleotide sequence of any one of SEQ ID NO: 1-4, placed under the control of appropriate regulatory elements.

The invention also relates to a pharmaceutical composition intended for the prevention of or treatment of subjects affected by a dysfunction of lipophilic substances transport or by a pathology located on the chromosome locus 2q34 such as for example the lamellar ichthyosis, the polymorphic congenital cataract, or insulin-dependant diabete mellitus, comprising a recombinant vector according to the invention, in combination with one or more physiologically compatible vehicle and/or excipient.

According to another aspect, the subject of the invention is also a preventive or curative therapeutic method of treating diseases caused by a deficiency of lipophilic substances transport or of a pathology located on the chromosome locus 2q34 such as for example the lamellar ichthyosis, the polymorphic congenital cataract, or insulin-dependant diabete mellitus, such a method comprising administering to a patient a nucleic acid encoding one ABCA12 polypeptide isoform according to the invention, said nucleic acid being combined with one or more physiologically appropriate vehicles and/or excipients.

The invention relates to a pharmaceutical composition for the prevention and/or treatment of a patient or subject affected by a dysfunction of the transport of lipophilic substances or by a pathology located on the chromosome locus 2q34 such as for example the lamellar ichthyosis, the polymorphic congenital cataract, or insulin-dependant diabete mellitus, comprising a therapeutically effective quantity of a polypeptide having an amino acid sequence selected from SEQ ID NO: 5 or 6, combined with one or more physiologically appropriate vehicles and/or excipients.

According to a specific embodiment, a method of introducing at least a nucleic acid according to the invention into a host cell, in particular a host cell obtained from a mammal, in vivo, comprises a step during which a preparation comprising a pharmaceutically compatible vector and a “naked” nucleic acid according to the invention, placed under the control of appropriate regulatory sequences, is introduced by local injection at the level of the chosen tissue, for example a smooth muscle tissue, the “naked” nucleic acid being absorbed by the cells of this tissue.

According to yet another aspect, the subject of the invention is also a preventive or curative therapeutic method of treating diseases caused by a deficiency of the ABCA12 gene and/or of lipophilic substances transport and/or located on the chromosome locus 2q34 such as for example the lamellar ichthyosis, the polymorphic congenital cataract, or insulin-dependant diabete mellitus, such a method comprising administering to a patient a therapeutically effective quantity of one of the ABCA12 polypeptide isoform according to the invention, said polypeptide being combined with one or more physiologically appropriate vehicles and/or excipients.

The invention also provides methods for screening small molecules and compounds that act on any one of ABCA12 protein isoforms to identify agonists and antagonists of such polypeptides that can restore or promote improved lipophilic substances transport to effectively cure and or prevent dysfunctions thereof or that can cure any pathology located on the chromosome locus 2q34 such as for example the lamellar ichthyosis, the polymorphic congenital cataract, or insulin-dependant diabete mellitus. These methods are useful to identify small molecules and compounds for therapeutic use in the treatment of diseases due to a deficiency of lipophilic substances transport or any pathology located on the chromosome locus 2q34 such as for example the lamellar ichthyosis, the polymorphic congenital cataract, or insulin-dependant diabete mellitus.

Therefore, the invention also relates to the use of any one of ABCA12 polypeptides or a cell expressing any one of ABCA12 polypeptides according to the invention, for screening active ingredients for the prevention and/or treatment of diseases resulting of a deficiency of lipophilic substances transport or located on the chromosome locus 2q34 such as for example the lamellar ichthyosis, the polymorphic congenital cataract, or insulin-dependant diabete mellitus.

The invention also relates to a method of screening a compound or small molecule, an agonist or antagonist of any one of ABCA12 polypeptides, said method comprising the following steps:

a) preparing a membrane vesicle comprising any one of ABCA12 polypeptides and a lipid substrate comprising a detectable marker;

b) incubating the vesicle obtained in step a) with an agonist or antagonist candidate compound;

c) qualitatively and/or quantitatively measuring release of the lipid substrate comprising a detectable marker; and

d) comparing the release measurement obtained in step b) with a measurement of release of a labelled lipid substrate by a vesicle that has not been previously incubated with the agonist or antagonist candidate compound.

In a first specific embodiment, the ABCA12 polypeptides comprise SEQ ID NO: 5 or 6, respectively.

The invention also relates to a method of screening a compound or small molecule, an agonist or antagonist of any one of ABCA12 polypeptides, said method comprising the following steps:

a) obtaining a cell, for example a cell line, that, either naturally or after transfecting the cell with any one of ABCA12 encoding nucleic acids, is capable of expressing corresponding ABCA12 polypeptides;

b) incubating the cell of step a) in the presence of an anion labelled with a detectable marker;

c) washing the cell of step b) in order to remove the excess of the labelled anion which has not penetrated into these cells;

d) incubating the cell obtained in step c) with an agonist or antagonist candidate compound for the any one of ABCA12 polypeptides;

e) measuring efflux of the labelled anion; and

f) comparing the value of efflux of the labelled anion determined in step e) with a value of efflux of a labelled anion measured with cell which has not been previously incubated in the presence of the agonist or antagonist candidate compound for any one of the ABCA12 polypeptides.

### BRIEF DESCRIPTION OF THE DRAWINGS

FIG. 1: represents the physical map of the portion of chromosome 2q34 region containing the ABCA12 gene. Locations of the microsatellite markers D2S317, D2S143, D2S137, D2S128, D2S1371, and D2S164 are indicated. Linkages of polymorphic congenital cataract, ichthyosis, and diabetes mellitus, insulin dependant on the human chromosome locus 2q34 are also indicated.

FIG. 2: represents the nucleotide sequence of one ABCA12 cDNA having SEQ ID NO:1. Start codon, stop codon and polyadenylation signals are displayed in bold letters. Primers and reverse primers are underlined and double-underlined, respectively.

FIG. 3: represents the nucleotide sequence of the ABCA12 cDNA having SEQ ID NO:2. Start codon, stop codon and polyadenylation signals are displayed in bold letters.

FIG. 4: represents the nucleotide sequence of the ABCA12 cDNA having SEQ ID NO:3. Start codon, stop codon and polyadenylation signals are displayed in bold letters.

FIG. 5: represents the nucleotide sequence of the ABCA12 cDNA having SEQ ID NO:4. Start codon, stop codon and polyadenylation signals are displayed in bold letters.

FIG. 6: represents the amino acid sequence of the ABCA12 protein longer isoform, having SEQ ID NO: 5. Start codon, stop codon and polyadenylation signals are displayed in bold letters.

FIG. 7: represents the amino acid sequence of the ABCA12 protein short isoform, having SEQ ID NO: 6. Start codon, stop codon and polyadenylation signals are displayed in bold letters.

### DETAILED DESCRIPTION OF THE INVENTION

General Definitions

The present invention contemplates isolation of human genes encoding ABCA12 polypeptides of the invention, including full and short length isoforms, or naturally occurring forms of ABCA12 and any antigenic fragments thereof from any animal, particularly mammalian or avian, and more particularly human source.

In accordance with the present invention, conventional molecular biology, microbiology, and recombinant DNA techniques within the skill of the art are used. Such techniques are fully explained in the literature (Sambrook et al., 1989, Molecular cloning a laboratory manual. 2ed. Cold Spring Harbor Laboratory, Cold spring Harbor, N.Y.; Glover, 1985, DNA Cloning: A pratical approach, volumes I and II oligonucleotide synthesis, MRL Press, LTD., Oxford, U.K.; Hames and Higgins, 1985, Transcription and translation; Hames and Higgins, 1984, Animal Cell Culture; Freshney, 1986, Immobilized Cells And Enzymes, IRL Press; and Perbal, 1984, A practical guide to molecular cloning).

As used herein, the term “gene” refers to an assembly of nucleotides that encode a polypeptide, and includes cDNA and genomic DNA nucleic acids.

The term “isolated” for the purposes of the present invention designates a biological material (nucleic acid or protein) which has been removed from its original environment (the environment in which it is naturally present).

For example, a polynucleotide present in the natural state in a plant or an animal is not isolated. The same nucleotide separated from the adjacent nucleic acids in which it is naturally inserted in the genome of the plant or animal is considered as being “isolated”.

Such a polynucleotide may be included in a vector and/or such a polynucleotide may be included in a composition and remains nevertheless in the isolated state because of the fact that the vector or the composition does not constitute its natural environment.

The term “purified” does not require the material to be present in a form exhibiting absolute purity, exclusive of the presence of other compounds. It is rather a relative definition.

A polynucleotide is in the “purified” state after purification of the starting material or of the natural material by at least one order of magnitude, preferably 2 or 3 and preferably 4 or 5 orders of magnitude.

For the purposes of the present description, the expression “nucleotide sequence” may be used to designate either a polynucleotide or a nucleic acid. The expression “nucleotide sequence” covers the genetic material itself and is therefore not restricted to the information relating to its sequence.

The terms “nucleic acid”, “polynucleotide”, “oligonucleotide” or “nucleotide sequence” cover RNA, DNA, or cDNA sequences or alternatively RNA/DNA hybrid sequences of more than one nucleotide, either in the single-stranded form or in the duplex, double-stranded form.

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA. The sequence of nucleotides that encodes a protein is called the sense sequence or coding sequence.

The term “nucleotide” designates both the natural nucleotides (A, T, G, C) as well as the modified nucleotides that comprise at least one modification such as (1) an analog of a purine, (2) an analog of a pyrimidine, or (3) an analogous sugar, examples of such modified nucleotides being described, for example, in the PCT application No. WO 95/04 064.

For the purposes of the present invention, a first polynucleotide is considered as being “complementary” to a second polynucleotide when each base of the first nucleotide is paired with the complementary base of the second polynucleotide whose orientation is reversed. The complementary bases are A and T (or A and U), or C and G.

“Heterologous” DNA refers to DNA not naturally located in the cell, or in a chromosomal site of the cell. Preferably, the heterologous DNA includes a gene foreign to the cell.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50 :667)). Such proteins (and their encoding genes) have sequence homology, as reflected by their high degree of sequence similarity.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and not a common evolutionary origin.

In a specific embodiment, two DNA sequences are “substantially homologous” or “substantially similar” when at least about 50% (preferably at least about 75%, and more preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art (See, e.g., Maniatis et al., supra; Glover et al. 1985. DNA Cloning: A practical approach, volumes I and II oligonucleatide synthesis, MRL Press, Ltd, Oxford, U.K.; Hames and Higgins, 1985. Transcription and Translation).

Similarly, in a particular embodiment, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program.

The “percentage identity” between two nucleotide or amino acid sequences, for the purposes of the present invention, may be determined by comparing two sequences aligned optimally, through a window for comparison.

The portion of the nucleotide or polypeptide sequence in the window for comparison may thus comprise additions or deletions (for example “gaps”) relative to the reference sequence (which does not comprise these additions or these deletions) so as to obtain an optimum alignment of the two sequences.

The percentage is calculated by determining the number of positions at which an identical nucleic base or an identical amino acid residue is observed for the two sequences (nucleic or peptide) compared, and then by dividing the number of positions at which there is identity between the two bases or amino acid residues by the total number of positions in the window for comparison, and then multiplying the result by 100 in order to obtain the percentage sequence identity.

The optimum sequence alignment for the comparison may be achieved using a computer with the aid of known algorithms contained in the package from the company WISCONSIN GENETICS SOFTWARE PACKAGE, GENETICS COMPUTER GROUP (GCG), 575 Science Doctor, Madison, Wis.

By way of illustration, it will be possible to produce the percentage sequence identity with the aid of the BLAST software (versions BLAST 1.4.9 of March 1996, BLAST 2.0.4 of February 1998 and BLAST 2.0.6 of September 1998), using exclusively the default parameters (Altschul et al, 1990,. Mol. Biol., 215:403-410; Altschul et al, 1997, Nucleic Acids Res., 25:3389-3402). Blast searches for sequences similar/homologous to a reference “request” sequence, with the aid of the Altschul et al. algorithm. The request sequence and the databases used may be of the peptide or nucleic types, any combination being possible.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

A gene encoding any one of ABCA12 polypeptides of the invention, whether genomic DNA or cDNA, can be isolated from any source, particularly from a human cDNA or genomic library. Methods for obtaining genes are well known in the art, as described above (see, e.g., Sambrook et al., 1989, Molecular cloning: a laboratory manual. 2ed. Cold Spring Harbor Laboratory, Cold spring Harbor, N.Y.).

Accordingly, any animal cell potentially can serve as the nucleic acid source for the molecular cloning of any one of ABCA12 genes. The DNA may be obtained by standard procedures known in the art from cloned DNA (e.g., a DNA “library”), and preferably is obtained from a cDNA library prepared from tissues with high level expression of the protein and/or the transcripts, by chemical synthesis, by cDNA cloning, or by the cloning of genomic DNA, or fragments thereof, purified from the desired cell (See, for example, Sambrook et al., 1989, Molecular cloning: a laboratory manual. 2ed. Cold Spring Harbor Laboratory, Cold spring Harbor, N.Y.; Glover, 1985, DNA Cloning: A Practical Approach, Volumes I and II Oligonucleotide Synthesis, MRL Press, Ltd., Oxford, U.K).

Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired ABCA12 gene may be accomplished in a number of ways. For example, if an amount of a portion of one of ABCA12 genes or its specific RNA, or a fragment thereof, is available and can be purified and labelled, the generated DNA fragments may be screened by nucleic acid hybridization to the labelled probe (Benton and Davis, Science (1977), 196:180; Grunstein et al., Proc.Natl. Acad. Sci. U.S.A. (1975) 72:3961). For example, a set of oligonucleotides corresponding to the partial coding sequence information obtained for the ABCA12 proteins can be prepared and used as probes for DNA encoding ABCA12, as was done in a specific example, infra, or as primers for cDNA or mRNA (e.g., in combination with a poly-T primer for RT-PCR). Preferably, a fragment is selected that is highly unique to the ABCA12 nucleic acids or polypeptides of the invention. Those DNA fragments with substantial homology to the probe will hybridize. As noted above, the greater the degree of homology, the more stringent hybridization conditions can be used. In a specific embodiment, various stringency hybridization conditions are used to identify homologous ABCA12 gene.

Further selection can be carried out on the basis of the properties of the gene, e.g., if the gene encodes a protein product having the isoelectric, electrophoretic, amino acid composition, or partial amino acid sequence of one of the ABCA12 proteins as disclosed herein. Thus, the presence of the gene may be detected by assays based on the physical, chemical, or immunological properties of its expressed product. For example, cDNA clones, or DNA clones which hybrid-select the proper mRNAs, can be selected which produce a protein that, e.g., has similar or identical electrophoretic migration, isoelectric focusing or non-equilibrium pH gel electrophoresis behaviour, proteolytic digestion maps, or antigenic properties as known for ABCA12.

The ABCA12 gene of the invention may also be identified by mRNA selection, i.e., by nucleic acid hybridization followed by in vitro translation. According to this procedure, nucleotide fragments are used to isolate complementary mRNAs by hybridization. Such DNA fragments may represent available, purified ABCA12 DNA, or may be synthetic oligonucleotides designed from the partial coding sequence information. Immunoprecipitation analysis or functional assays (e.g., tyrosine phosphatase activity) of the in vitro translation products of the products of the isolated mRNAs identifies the mRNA and, therefore, the complementary DNA fragments, that contain the desired sequences. In addition, specific mRNAs may be selected by adsorption of polysomes isolated from cells to immobilized antibodies specifically directed against any one of the ABCA12 polypeptides of the invention.

Radiolabeled ABCA12 cDNAs can be synthesized using the selected mRNA (from the adsorbed polysomes) as a template. The radiolabeled mRNA or cDNA may then be used as a probe to identify homologous ABCA12 DNA fragments from among other genomic DNA fragments.

“Variant” of a nucleic acid according to the invention will be understood to mean a nucleic acid which differs by one or more bases relative to the reference polynucleotide. A variant nucleic acid may be of natural origin, such as an allelic variant which exists naturally, or it may also be a normatural variant obtained, for example, by mutagenic techniques.

In general, the differences between the reference (generally, wild-type) nucleic acid and the variant nucleic acid are small such that the nucleotide sequences of the reference nucleic acid and of the variant nucleic acid are very similar and, in many regions, identical. The nucleotide modifications present in a variant nucleic acid may be silent, which means that they do not alter the amino acid sequences encoded by said variant nucleic acid.

However, the changes in nucleotides in a variant nucleic acid may also result in substitutions, additions or deletions in the polypeptide encoded by the variant nucleic acid in relation to the polypeptides encoded by the reference nucleic acid. In addition, nucleotide modifications in the coding regions may produce conservative or non-conservative substitutions in the amino acid sequence of the polypeptide.

Preferably, the variant nucleic acids according to the invention encode polypeptides which substantially conserve the same function or biological activity as the polypeptide of the reference nucleic acid or alternatively the capacity to be recognized by antibodies directed against the polypeptides encoded by the initial reference nucleic acid.

Some variant nucleic acids will thus encode mutated forms of the polypeptides whose systematic study will make it possible to deduce structure-activity relationships of the proteins in question. Knowledge of these variants in relation to the disease studied is essential since it makes it possible to understand the molecular cause of the pathology.

“Fragment” will be understood to mean a nucleotide sequence of reduced length relative to the reference nucleic acid and comprising, over the common portion, a nucleotide sequence identical to the reference nucleic acid. Such a nucleic acid “fragment” according to the invention may be, where appropriate, included in a larger polynucleotide of which it is a constituent. Such fragments comprise, or alternatively consist of, oligonucleotides ranging in length from 8, 10, 12, 15, 18, 20 to 25, 30, 40, 50, 70, 80, 100, 200, 500, 1000 or 1500 consecutive nucleotides of a nucleic acid according to the invention.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ of 55°, can be used, e.g., 5× SSC, 0.1% SDS, 0.25% milk, and no formamide; or 30% formamide, 5× SSC, 0.5% SDS. Moderate stringency hybridization conditions correspond to a higher Tₘ, e.g., 40% formamide, with 5× or 6× SCC. High stringency hybridization conditions correspond to the highest Tₘ, e.g., 50% formamide, 5× or 6× SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra). For hybridization with shorter nucleic acids, i.e., oligonucleotides, the position of mismatches becomes more important, and the length of the oligonucleotide determines its specificity (see Sambrook et al., supra). Preferably a minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

“High stringency hybridization conditions” for the purposes of the present invention will be understood to mean the following conditions:

1—Membrane competition and Prehybridization:

Mix: 40 μl salmon sperm DNA (10 mg/ml)

+40 μl human placental DNA (10 mg/ml)

Denature for 5 minutes at 96° C., then immerse the mixture in ice.

Remove the 2× SSC and pour 4 ml of formamide mix in the hybridization tube containing the membranes.

Add the mixture of the two denatured DNAs.

Incubation at 42° C. for 5 to 6 hours, with rotation.

2—Labeled Probe Competition:

Add to the labeled and purified probe 10 to 50 μl Cot I DNA, depending on the quantity of repeats.

Denature for 7 to 10 minutes at 95° C.

Incubate at 65° C. for 2 to 5 hours.

3—Hybridization:

Remove the prehybridization mix.

Mix 40 μl salmon sperm DNA +40 μl human placental DNA; denature for 5 min at 96° C., then immerse in ice.

Add to the hybridization tube 4 ml of formamide mix, the mixture of the two DNAs and the denatured labeled probe/Cot I DNA.

Incubate 15 to 20 hours at 42° C., with rotation.

4—Washes and Exposure:

One wash at room temperature in 2× SSC, to rinse.

Twice 5 minutes at room temperature 2× SSC and 0.1% SDS at 65° C.

Twice 15 minutes 0.1× SSC and 0.1% SDS at 65° C.

Envelope the membranes in clear plastic wrap and expose.

The hybridization conditions described above are adapted to hybridization, under high stringency conditions, of a molecule of nucleic acid of varying length from 20 nucleotides to several hundreds of nucleotides. It goes without saying that the hybridization conditions described above may be adjusted as a function of the length of the nucleic acid whose hybridization is sought or of the type of labeling chosen, according to techniques known to one skilled in the art. Suitable hybridization conditions may, for example, be adjusted according to the teaching contained in the manual by Hames and Higgins (1985, supra).

As used herein, the term “oligonucleotide” refers to a nucleic acid, generally of at least 15 nucleotides, that is hybridizable to a nucleic acid according to the invention. Oligonucleotides can be labelled, e.g., with 32P-nucleotides or nucleotides to which a label, such as biotin, has been covalently conjugated. In one embodiment, a labeled oligonucleotide can be used as a probe to detect the presence of a nucleic acid encoding an ABCA5-6, 9-10 polypeptide of the invention. In another embodiment, oligonucleotides (one or both of which may be labelled) can be used as PCR primers, either for cloning full lengths or fragments of any one of the ABCA5, ABCA6, ABCA9, and ABCA10 nucleic acids, or to detect the presence of nucleic acids encoding any one of the ABCA5, ABCA6, ABCA9, and ABCA10. In a further embodiment, an oligonucleotide of the invention can form a triple helix with any one of the ABCA12 DNA molecules. Generally, oligonucleotides are prepared synthetically, preferably on a nucleic acid synthesizer. Accordingly, oligonucleotides can be prepared with non-naturally occurring phosphoester analog bonds, such as thioester bonds, etc.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

“Regulatory region” means a nucleic acid sequence which regulates the expression of a nucleic acid. A regulatory region may include sequences which are naturally responsible for expressing a particular nucleic acid (a homologous region) or may include sequences of a different origin (responsible for expressing different proteins or even synthetic proteins). In particular, the sequences can be sequences of eukaryotic or viral genes or derived sequences which stimulate or repress transcription of a gene in a specific or non-specific manner and in an inducible or non-inducible manner. Regulatory regions include origins of replication, RNA splice sites, enhancers, transcriptional termination sequences, signal sequences which direct the polypeptide into the secretory pathways of the target cell, and promoters.

A regulatory region from a “heterologous source” is a regulatory region which is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

A “polypeptide” is a polymeric compound comprised of covalently linked amino acid residues. Amino acids have the following general structure:

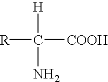

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

A “protein” is a polypeptide which plays a structural or functional role in a living cell.

The polypeptides and proteins of the invention may be glycosylated or unglycosylated.

“Homology” means similarity of sequence reflecting a common evolutionary origin. Polypeptides or proteins are said to have homology, or similarity, if a substantial number of their amino acids are either (1) identical, or (2) have a chemically similar R side chain. Nucleic acids are said to have homology if a substantial number of their nucleotides are identical.

“Isolated polypeptide” or “isolated protein” is a polypeptide or protein which is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

“Fragment” of a polypeptide according to the invention will be understood to mean a polypeptide whose amino acid sequence is shorter than that of the reference polypeptide and which comprises, over the entire portion with these reference polypeptides, an identical amino acid sequence. Such fragments may, where appropriate, be included in a larger polypeptide of which they are a part. Such fragments of a polypeptide according to the invention may have a length of 5, 10, 15, 20, 30 to 40, 50, 100, 200 or 300 amino acids.

“Variant” of a polypeptide according to the invention will be understood to mean mainly a polypeptide whose amino acid sequence contains one or more substitutions, additions or deletions of at least one amino acid residue, relative to the amino acid sequence of the reference polypeptide, it being understood that the amino acid substitutions may be either conservative or nonconservative.

A “variant” of a polypeptide or protein is any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. Variants also include a related protein having substantially the same biological activity, but obtained from a different species.

The skilled artisan can produce variants having single or multiple amino acid substitutions, deletions, additions, or replacements. These variants may include, inter alia: (a) variants in which one or more amino acid residues are substituted with conservative or non-conservative amino acids, (b) variants in which one or more amino acids are added to the polypeptide or protein, (c) variants in which one or more of the amino acids includes a substituent group, and (d) variants in which the polypeptide or protein is fused with another polypeptide such as serum albumin. The techniques for obtaining these variants, including genetic (suppressions, deletions, mutations, etc.), chemical, and enzymatic techniques, are known to persons having ordinary skill in the art.

If such allelic variations, analogues, fragments, derivatives, mutants, and modifications, including alternative mRNA splicing forms and alternative post-translational modification forms result in derivatives of the polypeptide which retain any of the biological properties of the polypeptide, they are intended to be included within the scope of this invention.

A “vector” is a replicon, such as plasmid, virus, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

The present invention also relates to cloning vectors containing genes encoding analogs and derivatives any of the ABCA12 polypeptides of the invention, that have the same or homologous functional activity as that of ABCA12 polypeptides, and homologs thereof from other species. The production and use of derivatives and analogs related to ABCA12 are within the scope of the present invention. In a specific embodiment, the derivatives or analogs are functionally active, i.e., capable of exhibiting one or more functional activities associated with a full-length, wild-type ABCA12 polypeptides of the invention.

ABCA12 derivatives can be made by altering encoding nucleic acid sequences by substitutions, additions or deletions that provide for functionally equivalent molecules. Preferably, derivatives are made that have enhanced or increased functional activity relative to native ABCA12. Alternatively, such derivatives may encode soluble fragments of the ABCA12 extracellular domains that have the same or greater affinity for the natural ligand of ABCA12 polypeptides of the invention. Such soluble derivatives may be potent inhibitors of ligand binding to ABCA12.

Due to the degeneracy of nucleotide coding sequences, other DNA sequences which encode substantially same amino acid sequences as that of ABCA12 genes may be used in the practice of the present invention. These include but are not limited to allelic genes, homologous genes from other species, and nucleotide sequences comprising all or portions of ABCA12 genes which are altered by the substitution of different codons that encode the same amino acid residue within the sequence, thus producing a silent change. Likewise, the ABCA12 derivatives of the invention include, but are not limited to, those containing, as a primary amino acid sequence, all or part of the amino acid sequence of any one of the ABCA12 proteins including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point.

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free CONH₂ can be maintained.

Amino acid substitutions may also be introduced to substitute an amino acid with a particularly preferable property. For example, a Cys may be introduced as a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces b-turns in the protein's structure.

The genes encoding ABCA12 derivatives and analogs of the invention can be produced by various methods known in the art. The manipulations which result in their production can occur at the gene or protein level. For example, the cloned ABCA12 sequences can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. Production of a gene encoding a derivative or analog of the ABCA12 should ensure that the modified gene remains within the same translational reading frame as the ABCA12 genes, uninterrupted by translational stop signals, in the region where the desired activity is encoded.

Additionally, the ABCA12-encoding nucleic acids can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Preferably, such mutations enhance the functional activity of the mutated ABCA12 gene products. Any technique for mutagenesis known in the art may be used, including inter alia, in vitro site-directed mutagenesis (Hutchinson et al., (1978) Biol. Chem. 253:6551; Zoller and Smith, (1984) DNA, 3:479-488; Oliphant et al., (1986) Gene 44:177; Hutchinson et al., (1986) Proc. Natl. Acad. Sci. U.S.A. 83:710; Huygen et al., (1996), Nature Medicine, 2(8):893-898) and use of TAB® linkers (Pharmacia). PCR techniques are preferred for site-directed mutagenesis (Higuchi, 1989, “Using PCR to Engineer DNA”, in PCR Technology: Principles and Applications for DNA Amplification, H. Erlich, ed., Stockton Press, Chapter 6, pp. 61-70).

Identified and isolated ABCA12 genes may then be inserted into an appropriate cloning vector. A large number of vector-host systems known in the art may be used. Possible vectors include, but are not limited to plasmids or modified viruses, but the vector system must be compatible with the host cell used. Examples of vectors include, but are not limited to, Escherichia coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., Escherichia coli, and facile purification for subsequent insertion into an appropriate expression cell line, if such is desired. For example, a shuttle vector, which is a vector that can replicate in more than one type of organism, can be prepared for replication in both Escherichia coli and Saccharomyces cerevisiae by linking sequences from an Escherichia coli plasmid with sequences form the yeast 2m plasmid.

In an alternative method, the desired gene may be identified and isolated after insertion into a suitable cloning vector in a “shot gun” approach. Enrichment for the desired gene, for example, by size fractionation, can be done before insertion into the cloning vector.

The nucleotide sequence coding for ABCA12 polypeptides or antigenic fragments, derivatives or analogs thereof, or functionally active derivatives, including chimeric proteins thereof, may be inserted into an appropriate expression vector, i.e., a vector which contains the necessary elements for the transcription and translation of the inserted protein-coding sequence. Such elements are termed herein a “promoter.” Thus, nucleic acids encoding ABCA12 polypeptides of the invention are operationally associated with a promoter in an expression vector of the invention. Both cDNA and genomic sequences can be cloned and expressed under control of such regulatory sequences. An expression vector also preferably includes a replication origin.

The necessary transcriptional and translational signals can be provided on a recombinant expression vector, or they may be supplied by a native gene encoding ABCA12 and/or its flanking region.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant ABCA12 protein of the invention, or functional fragments, derivatives, chimeric constructs, or analogs thereof, may be expressed chromosomally, after integration of the coding sequence by recombination. In this regard, any of a number of amplification systems may be used to achieve high levels of stable gene expression (See Sambrook et al., 1989, supra).

The cell into which the recombinant vector comprising the nucleic acid encoding any one of the ABCA12 polypeptides according to the invention is cultured in an appropriate cell culture medium under conditions that provide for expression of any one of the ABCA12 polypeptides by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of ABCA12 polypeptides may be controlled by any promoter/enhancer element known in the art, but these regulatory elements must be functional in the host selected for expression. Promoters which may be used to control ABCA12 gene expression include, but are not limited to, the SV40 early promoter region (Benoist and Chambon, 1981 Nature 290:304-310), the promoter contained in the 3′ long terminal repeat of Rous sarcoma virus (Yamamoto, et al., 1980, Cell, 22:787-797), the herpes thymidine kinase promoter (Wagner et al., 1981, Proc. Natl. Acad. Sci. U.S.A., 78:1441-1445), the regulatory sequences of the metallothionein gene (Brinster et al., 1982, Nature, 296:39-42); prokaryotic expression vectors such as the β-lactamase promoter (Villa-Kamaroff, et al., 1978, Proc. Natl. Acad. Sci. U.S.A., 75:3727-3731), or the tac promoter (DeBoer, et al., 1983, Proc. Natl. Acad. Sci. U.S.A., 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984, Cell, 38:639-646; Ornitz et al., 1986, Cold Spring Harbor Symp. Quant. Biol., 50:399-409; MacDonald, 1987); insulin gene control region which is active in pancreatic beta cells (Hanahan, 1985, Nature, 315:115-122), immunoglobulin gene control region which is active in lymphoid cells (Grosschedl et al., 1984, Cell, 38:647-658; Adames et al., 1985, Nature, 318:533-538; Alexander et al., 1987, Mol. Cell. Biol., 7:1436-1444), mouse mammary tumor virus control region which is active in testicular, breast, lymphoid and mast cells (Leder et al., 1986, Cell, 45:485-495), albumin gene control region which is active in liver (Pinkert et al., 1987, Genes and Devel., 1:268-276), alpha-fetoprotein gene control region which is active in liver (Krumlauf et al., 1985, Mol. Cell. Biol., 5:1639-1648; Hammer et al., 1987, Science, 235:53-58), alpha 1-antitrypsin gene control region which is active in the liver (Kelsey et al., 1987, Genes and Devel., 1:161-171) beta-globin gene control region which is active in myeloid cells (Mogram et al., 1985, Nature, 315:338-340; Kollias et al., 1986, Cell, 46:89-94), myelin basic protein gene control region which is active in oligodendrocyte cells in the brain (Readhead et al., 1987, Cell, 48:703-712), myosin light chain-2 gene control region which is active in skeletal muscle (Sani, 1985, Nature, 314:283-286), and gonadotropic releasing hormone gene control region which is active in the hypothalamus (Mason et al., 1986, Science, 234:1372-1378).

Expression vectors containing a nucleic acid encoding one of ABCA12 polypeptides of the invention can be identified by five general approaches: (a) polymerase chain reaction (PCR) amplification of the desired plasmid DNA or specific mRNA, (b) nucleic acid hybridization, (c) presence or absence of selection marker gene functions, (d) analyses with appropriate restriction endonucleases, and (e) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., b-galactosidase activity, thymidine kinase activity, resistance to antibiotics, transformation phenotype, occlusion body formation in baculovirus, etc.) caused by the insertion of foreign genes in the vector. In another example, if the nucleic acid encoding any one of the ABCA12 polypeptides is inserted within the “selection marker” gene sequence of the vector, recombinants containing ABCA12 nucleic acids inserts can be identified by the absence of the ABCA12 genes functions. In the fourth approach, recombinant expression vectors are identified by digestion with appropriate restriction enzymes. In the fifth approach, recombinant expression vectors can be identified by assaying for the activity, biochemical, or immunological characteristics of the gene product expressed by the recombinant, provided that the expressed protein assumes a functionally active conformation.

A wide variety of host/expression vector combinations may be employed in expressing the nucleic acids of this invention. Useful expression vectors, for example, may consist of segments of chromosomal, non-chromosomal and synthetic DNA sequences. Suitable vectors include derivatives of SV40 and known bacterial plasmids, e.g., Escherichia coli plasmids col E1, pCR1, pBR322, pMal-C2, pET, pGEX (Smith et al., 1988, Gene, 67:31-40), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamH1 cloning site; Summers), pVL1393 (BamH1, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamH1 recognition site begins with the initiation codon; Summers), pAc700 and pAc702 (same as pAc700, with different reading frames), pAc360 (BamH1 cloning site 36 base pairs downstream of a polyhedrin initiation codon; Invitrogen(195)), and pBlueBacHisA, B, C (three different reading frames, with BamH1, BglII, PstI, NcoI, and HindIII cloning site, an N-terminal peptide for ProBond purification, and blue/white recombinant screening of plaques; Invitrogen (220) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; See, Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhaI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible methallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SmaI cloning site, TK- and b-gal selection), pMJ601 (SalI, SmaI, AflI, NarI, BspMII, BamHI, ApaI, NheI, SacII, KpnI, and HindIII cloning site; TK- and b-gal selection), and pTKgptF1S (EcoRI, PstI, SalI, AccI, HindII, SbaI, BamHI, and Hpa cloning site, TK or XPRT selection).

Yeast expression systems can also be used according to the invention to express any one of the ABCA12 polypeptides. For example, the non-fusion pYES2 vector (XbaI, SphI, ShoI, NotI, GstXI, EcoRI, BstXI, BamH1, SacI, KpnI, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Different host cells have characteristic and specific mechanisms for the translational and post-translational processing and modification (e.g., glycosylation, cleavage for example of the signal sequence) of proteins. Appropriate cell lines or host systems can be chosen to ensure the desired modification and processing of the foreign protein expressed. For example, expression in a bacterial system can be used to produce an nonglycosylated core protein product. However, the transmembrane ABCA12 proteins expressed in bacteria may not be properly folded. Expression in yeast can produce a glycosylated product. Expression in eukaryotic cells can increase the likelihood of “native” glycosylation and folding of a heterologous protein. Moreover, expression in mammalian cells can provide a tool for reconstituting, or constituting, ABCA12 activities. Furthermore, different vector/host expression systems may affect processing reactions, such as proteolytic cleavages, to a different extent.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (Wu et al., 1992, J. Biol. Chem., 267:963-967; Wu and Wu, 1988, J. Biol. Chem., 263:14621-14624; Hartmut et al., Canadian Patent Application No. 2,012,311, filed Mar. 15, 1990).

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

A recombinant marker protein expressed as an integral membrane protein can be isolated and purified by standard methods. Generally, the integral membrane protein can be obtained by lysing the membrane with detergents, such as but not limited to, sodium dodecyl sulfate (SDS), Triton X-100 polyoxyethylene ester, Ipagel/nonidet P-40 (NP-40) (octylphenoxy)-polyethoxyethanol, digoxin, sodium deoxycholate, and the like, including mixtures thereof. Solubilization can be enhanced by sonication of the suspension. Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

Alternatively, a nucleic acid or vector according to the invention can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner, et. al. (1987. PNAS 84/7413); Mackey, et al. (1988. Proc. Natl. Acad. Sci. USA 85 :8027-8031); Ulmer et al. (1993. Science 259 :1745-1748). The use of cationic lipids may promote encapsulation of negatively charged nucleic acids, and also promote fusion with negatively charged cell membranes (Felgner et al., 1989, Science, 337:387-388)). Particularly useful lipid compounds and compositions for transfer of nucleic acids are described in International Patent Publications WO95/18863 and WO96/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931).

It is also possible to introduce the vector in vivo as a naked DNA plasmid (see U.S. Pat. Nos. 5,693,622, 5,589,466 and 5,580,859). Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, Wu et al., 1992, supra; Wu and Wu, 1988, supra; Hartmut et al., Canadian Patent Application No. 2,012,311, filed Mar. 15, 1990; Williams et al., 1991, Proc. Natl. Acad. Sci. USA 88:2726-2730). Receptor-mediated DNA delivery approaches can also be used (Curiel et al., 1992, Hum. Gene Ther. 3:147-154; Wu and Wu, 1987, J. Biol. Chem. 262:4429-4432).

“Pharmaceutically acceptable vehicle or excipient” includes diluents and fillers which are pharmaceutically acceptable for method of administration, are sterile, and may be aqueous or oleaginous suspensions formulated using suitable dispersing or wetting agents and suspending agents. The particular pharmaceutically acceptable carrier and the ratio of active compound to carrier are determined by the solubility and chemical properties of the composition, the particular mode of administration, and standard pharmaceutical practice.

Any nucleic acid, polypeptide, vector, or host cell of the invention will preferably be introduced in vivo in a pharmaceutically acceptable vehicle or excipient. The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “excipient” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as excipients, particularly for injectable solutions. Suitable pharmaceutical excipients are described in “Remington's Pharmaceutical Sciences” by E. W. Martin.

Naturally, the invention contemplates delivery of a vector that will express a therapeutically effective amount of any one of ABCA12 polypeptides for gene therapy applications. The phrase “therapeutically effective amount” is used herein to mean an amount sufficient to reduce by at least about 15 percent, preferably by at least 50 percent, more preferably by at least 90 percent, and still more preferably prevent, a clinically significant deficit in the activity, function and response of the host. Alternatively, a therapeutically effective amount is sufficient to cause an improvement in a clinically significant condition in the host.

cDNA Molecules Encoding Full and Short Length of the ABCA12 Proteins

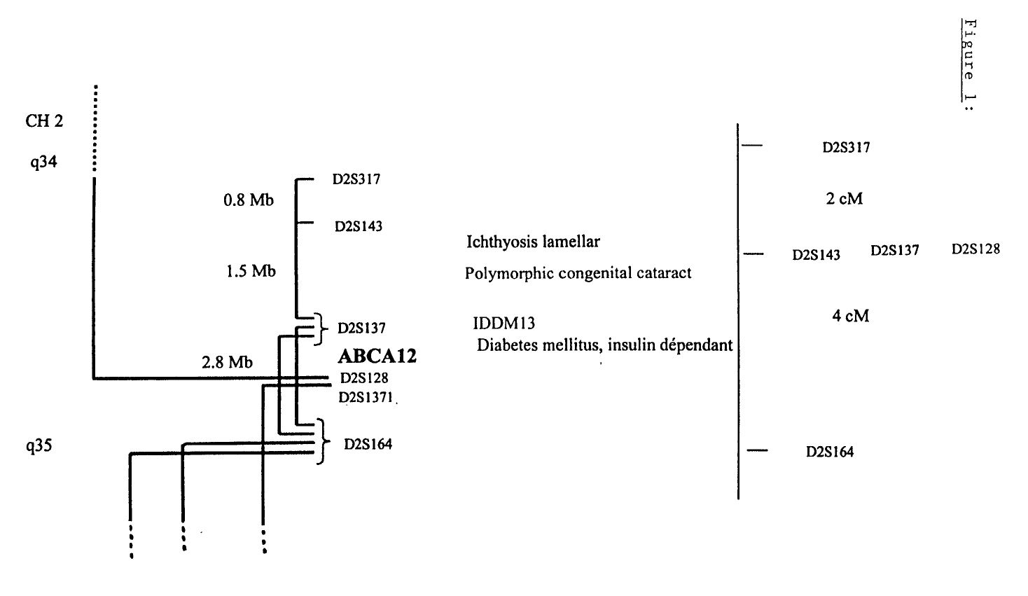

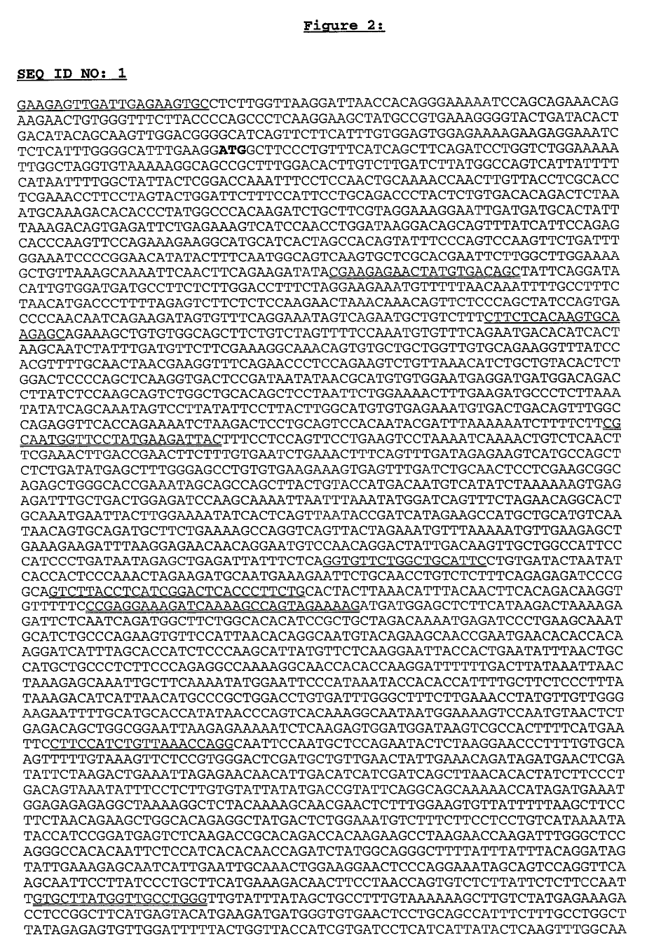

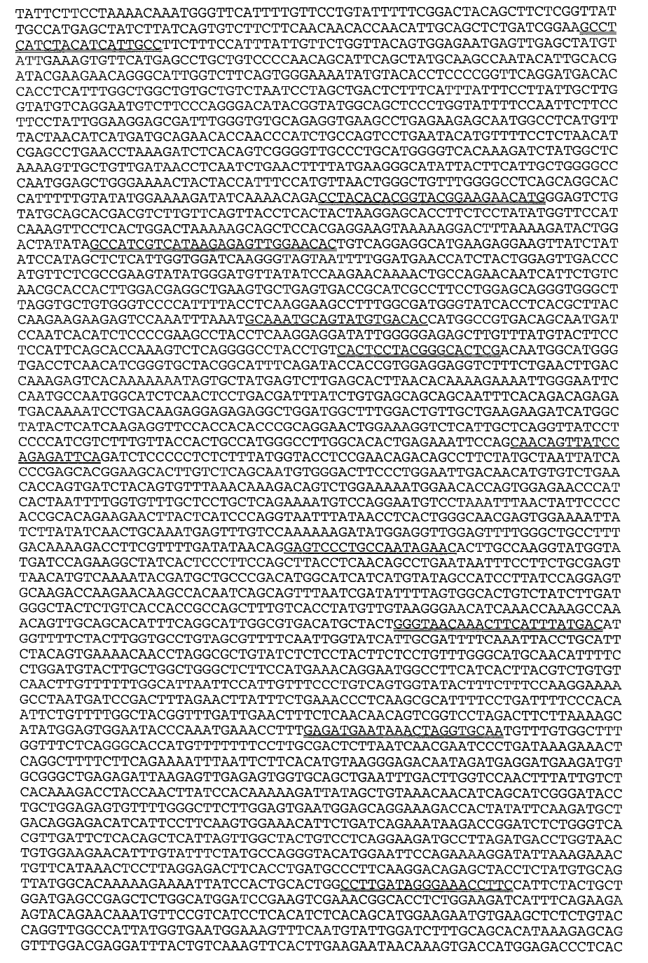

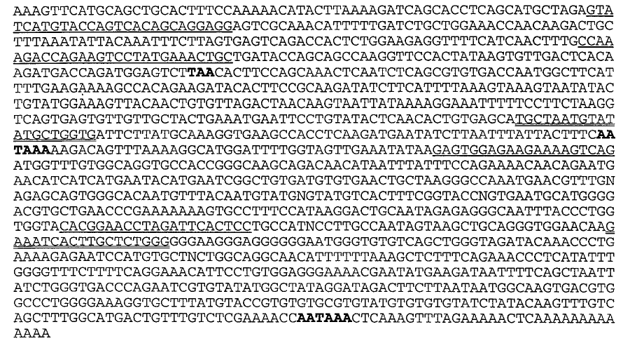

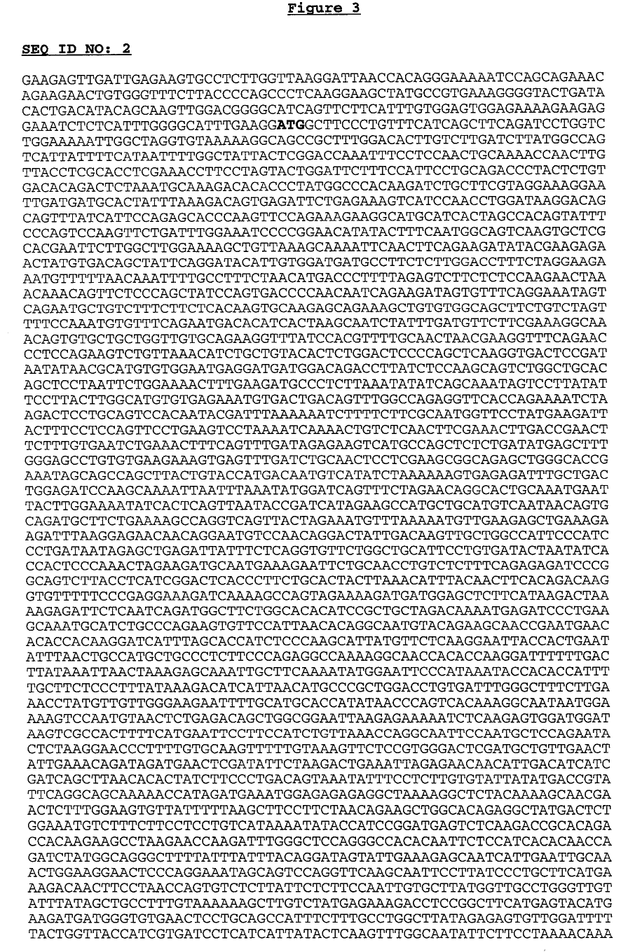

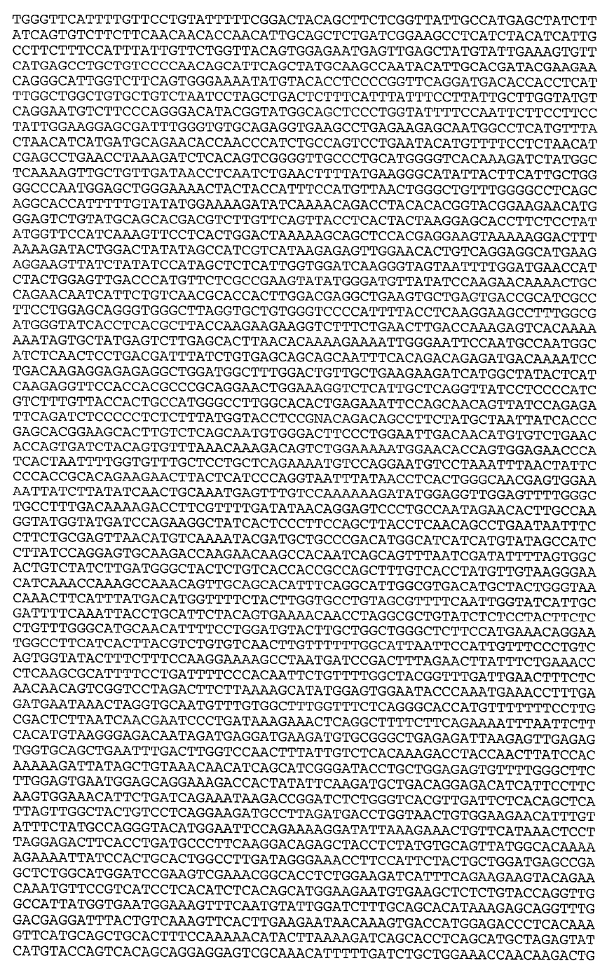

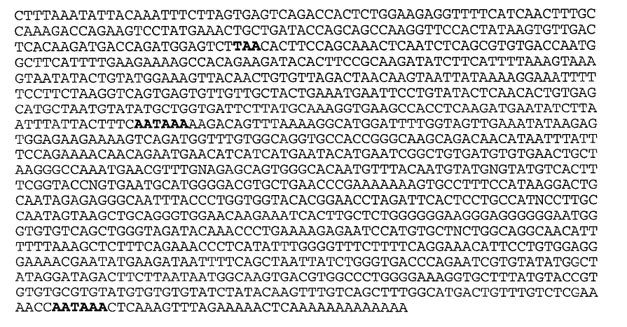

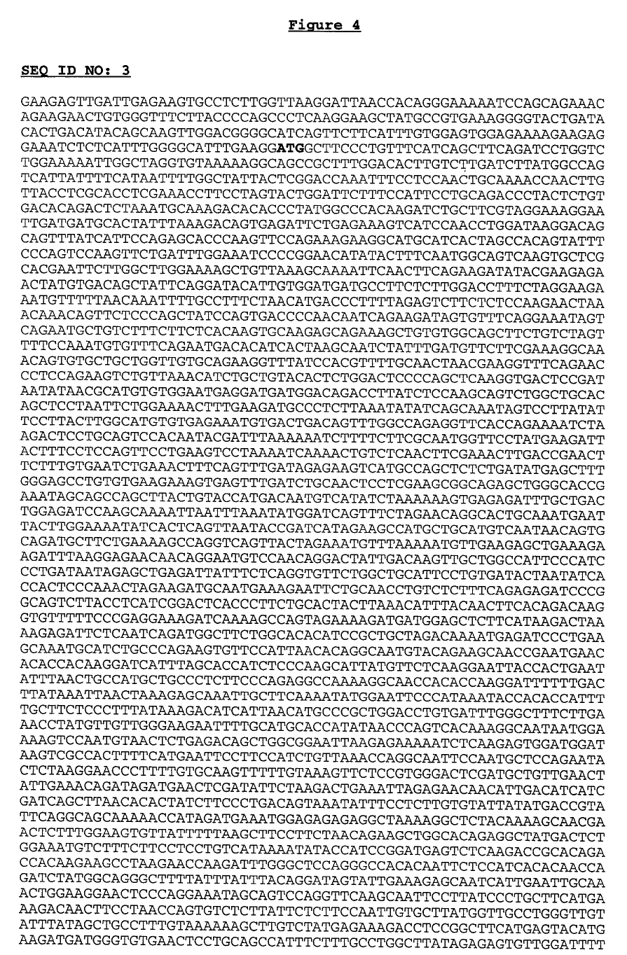

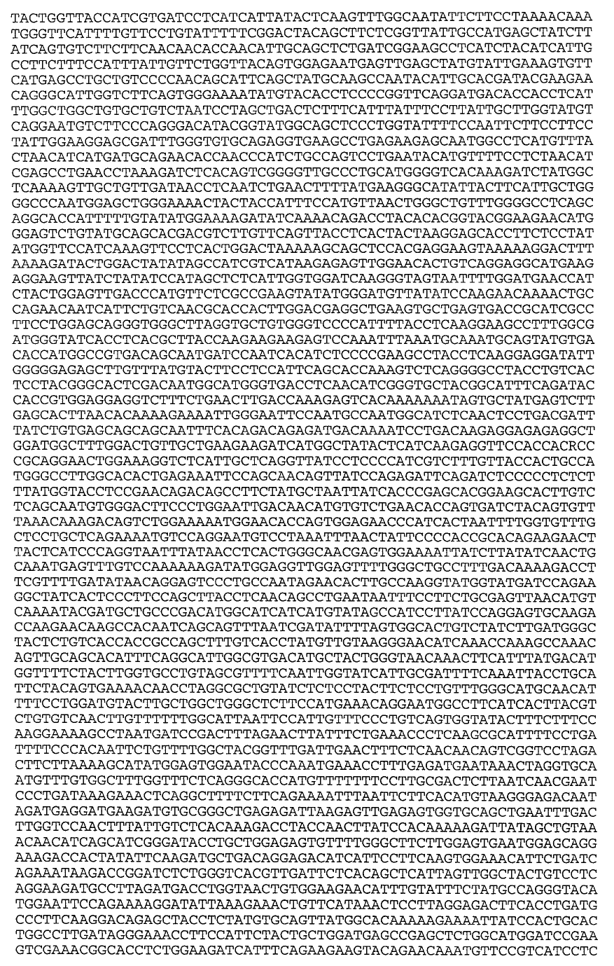

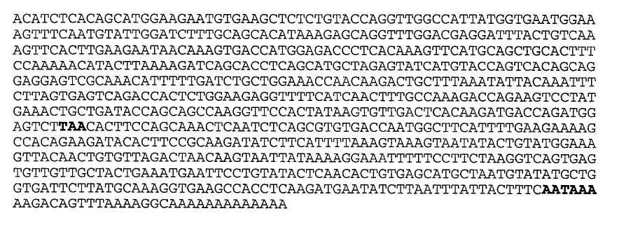

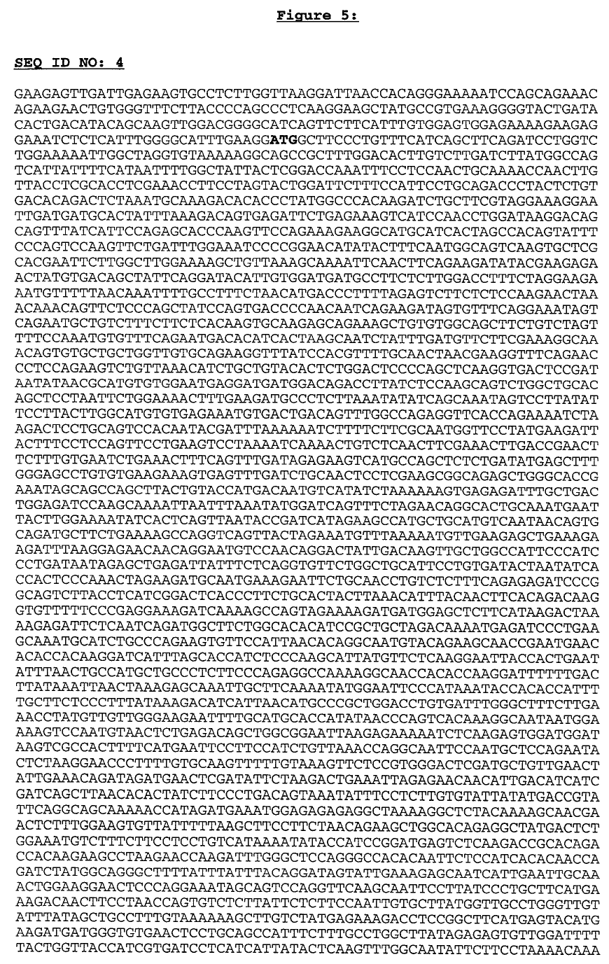

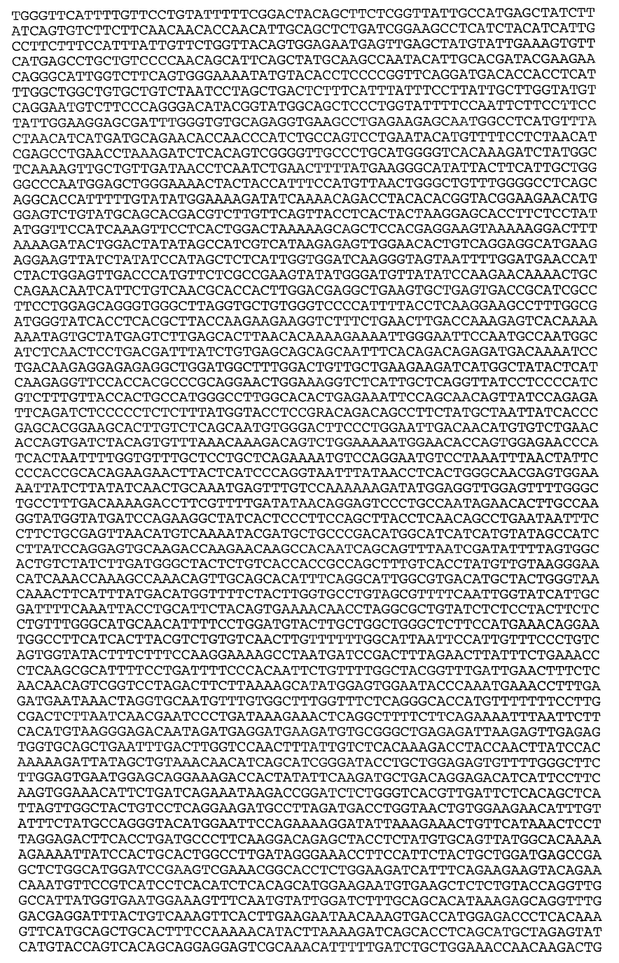

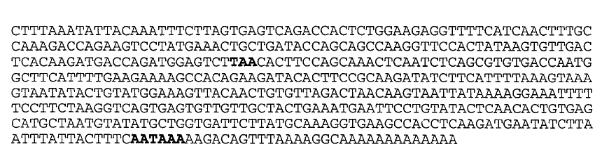

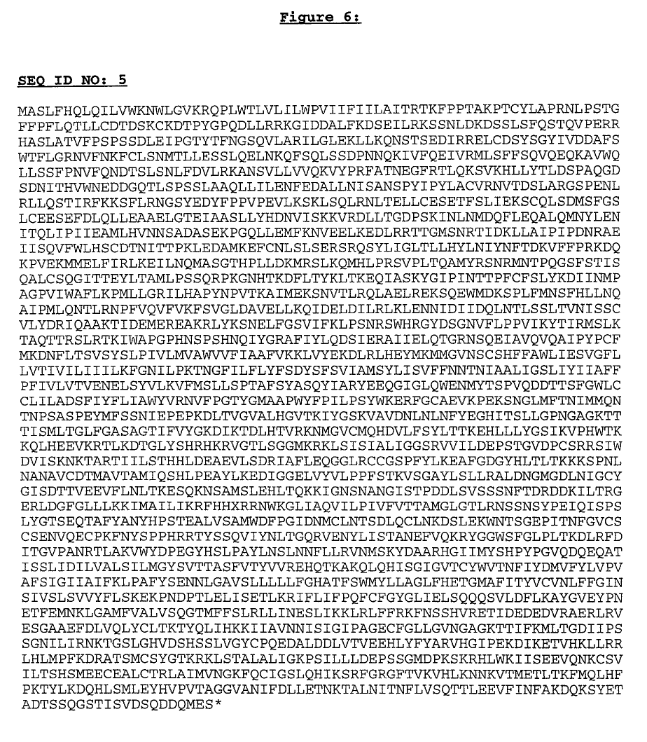

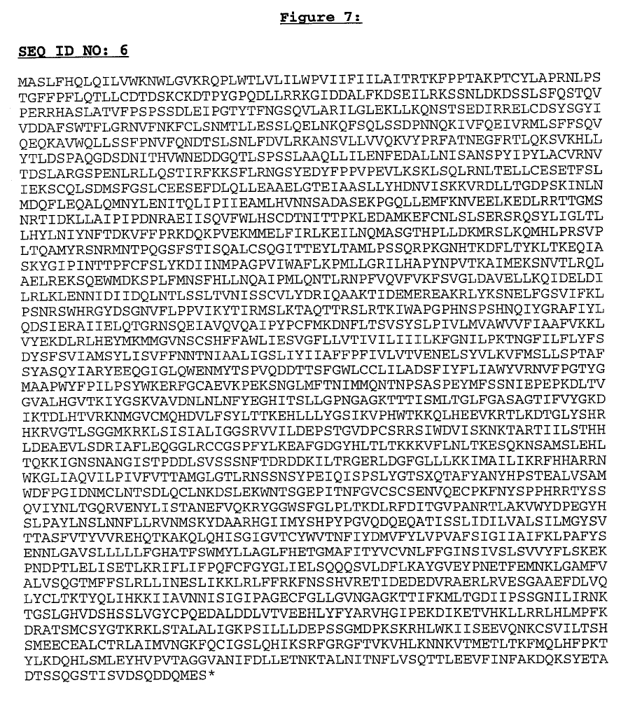

The applicants have identified a novel human ABCA-like gene, designated ABCA12, and determined that this gene is located on the region of chromosome 2q34 (FIG. 1). The applicants have also identified various ABCA12 transcripts herein designated transcripts A-D and the full coding sequences (CDS) corresponding to the human ABCA12 gene which encodes two human corresponding protein isoforms.

Table 1 summarizes the ABCA12 mRNA length, the coding nucleotide sequence length, position of polyadenylation sites as well as the predicted proteins sizes.

TABLE 1
Characterization of the ABCA12 transcripts on the chromosome 2q34

| SEQ | | mRNA | | Position of the | Putative |
|---|---|---|---|---|---|
| ID | ABCA12 various | length | CDS | Polyadenylation | protein |
| NOS: | forms of transcripts | (bp) | (bp) | site AATAAA | (AA) |
| 1 | Transcript A | 9112 | 7788 | 9074 | 2595 |
| 2 | Transcript B | 8875 | 7551 | 8837 | 2516 |
| 3 | Transcript C | 8350 | 7788 | 8315 | 2595 |
| 4 | Transcript D | 8113 | 7551 | 8078 | 2516 |

Transcript A of the human novel ABCA12 gene consists of 9112 nucleotides having the nucleotide sequence as set forth in SEQ ID NO: 1, and comprises a 7788 bp open reading frame beginning from the nucleotide at position 221 (base A of the ATG codon for initiation of translation) to the nucleotide at position 8008 (second base A of the TAA stop codon). Two putative polyadenylation signals (having the sequence AATAAA) are present, starting from the nucleotides at positions 8315 and 9074 of the sequence SEQ ID NO: 1.

According to the invention, the ABCA12 cDNA form A (SEQ ID NO: 1) contains a 7788 bp coding sequence which encodes a full length ABCA12 polypeptide of 2595 amino acids (aa) comprising the amino acid sequence of SEQ ID NO: 5.

Transcript B of the human novel ABCA12 gene consists of 8875 nucleotides as set forth in SEQ ID NO: 2, and comprises a 7551 bp open reading frame beginning from the nucleotide at position 221 (base A of the ATG codon for initiation of translation) to the nucleotide at position 7771 (second base A of the TAA stop codon). Putative polyadenylation signals (having the sequence AATAAA) are present, starting from the nucleotide at positions 8078 and 8837 of the sequence SEQ ID NO: 2.

According to the invention, the ABCA12 cDNA form B (SEQ ID NO: 2) contains a 7551 bp coding sequence which encodes a short length ABCA12 polypeptide of 2516 amino acids comprising the amino acid sequence of SEQ ID NO: 6.

Transcript C of the human novel ABCA12 gene consists of 8350 nucleotides as set forth in SEQ ID NO: 3, and comprises a 7788 bp open reading frame beginning from the nucleotide at position 221 (base A of the ATG codon for initiation of translation) to the nucleotide at position 8008 (second base A of the TAA stop codon). A putative polyadenylation signal (having the sequence AATAAA) is present, starting from the nucleotide at position 8315 of the sequence SEQ ID NO: 3.

According to the invention, the ABCA12 cDNA (SEQ ID NO: 3) contains a 7788 bp coding sequence which encodes a full length ABCA12 polypeptide of 2595 amino acids comprising the amino acid sequence of SEQ ID NO: 5.

Transcript D of the novel human ABCA12 gene consists of 8113 nucleotides as set forth in SEQ ID NO: 4, and comprises a 7551 bp open reading frame beginning from the nucleotide at position 221 (base A of the ATG codon for initiation of translation) to the nucleotide at position 7771 (second base A of the TAA stop codon). A putative polyadenylation signal (having the sequence AATAAA) is present, starting from the nucleotide at position 8078 of the sequence SEQ ID NO: 4.

According to the invention, the ABCA12 cDNA (SEQ ID NO: 4) contains a 7551 bp coding sequence which encodes a short length ABCA12 polypeptide of 2516 amino acids comprising the amino acid sequence of SEQ ID NO: 6.

The applicants have also determined that the ABCA12 gene has a specific expression pattern, suggesting that the corresponding protein isoforms may perform tissue-specialized functions (Example 3). In effect, electronic analysis of tissue distribution showed that the ABCA12 transcript matches with various ESTs of different tissue origin, suggesting a preferential expression in skin/epithelial tissues.

The applicants have further determined potential transcript sequences that should correspond to the full coding sequence (CDS) of the ABCA12 gene, which are particularly useful according to the invention for the production of various means of detection of the ABCA12 gene, or nucleotide expression products in a sample.

The present invention is thus directed to a nucleic acid comprising SEQ ID NOs: 1-4, or a complementary nucleotide sequence thereof.

The invention also relates to a nucleic acid comprising a nucleotide sequence as depicted in SEQ ID NO: 1-4 or a complementary nucleotide sequence thereof.

The invention also relates to a nucleic acid comprising at least eight consecutive nucleotides of SEQ ID NO: 1-4 or a complementary nucleotide sequence thereof.

The subject of the invention is also a nucleic acid having at least 80% nucleotide identity with a nucleic acid comprising any one of SEQ ID NO: 1-4, or a nucleic acid having a complementary nucleotide sequence thereof.

The invention also relates to a nucleic acid having at least 85%, preferably 90%, more preferably 95% and still more preferably 98% nucleotide identity with a nucleic acid comprising any one of SEQ ID NO:1-4, or a nucleic acid having a complementary nucleotide sequence thereof.

Another subject of the invention is a nucleic acid hybridizing, under high stringency conditions, with a nucleic acid comprising any one of SEQ ID NO: 1-4, or a nucleic acid having a complementary nucleotide sequence thereof.

The invention also relates to a nucleic acid encoding a polypeptide comprising an amino acid sequence of SEQ ID NO: 5 or 6.

The invention relates to a nucleic acid encoding a polypeptide comprising an amino acid sequence as depicted in SEQ ID NO:5 or 6.

The invention also relates to a polypeptide comprising amino acid sequence of SEQ ID NO: 5 or 6.

The invention also relates to a polypeptide comprising amino acid sequence as depicted in SEQ ID NO: 5 or 6.

The invention also relates to a polypeptide comprising an amino acid sequence having at least 80% amino acid identity with a polypeptide comprising an amino acid sequence of SEQ ID NO: 5 or 6, or a peptide fragment thereof.

The invention also relates to a polypeptide having at least 85%, preferably 90%, more preferably 95% and still more preferably 98% amino acid identity with a polypeptide comprising an amino acid sequence of SEQ ID NO: 5 or 6.

Preferably, a polypeptide according to the invention will have a length of 4, 5 to 10, 15, 18 or 20 to 25, 35, 40, 50, 70, 80, 100 or 200 consecutive amino acids of a polypeptide according to the invention comprising an amino acid sequence of SEQ ID NO: 5 or 6.

Like ABCA1 and ABCA4 transporters, which present 52% amino acid sequences identity, or ABCA5, ABCA6, ABCA9 and ABCA10 genes that present an identity ranging from 43 to 62% along the entire sequence, ABCA12 proteins also demonstrate high conservation as set forth in Table 2. Alignment of the long amino acid sequence of ABCA12 with amino acid sequences of ABCA4, ABCA7 (Kaminski et al., Biochem Biophys Res Commun, 2000, 278(3):782-9), ABCA5, ABCA9 (EP00403440) genes reveals an identity ranging from 28 to 36% along the entire sequence. The same kind of result is obtained with the short amino acid sequence of ABCA12.

TABLE 2
Homology/Identity percentages between the amino acid sequences
of ABCA1, ABCA4, ABCA7, ABCA5, ABCA9, and ABCA12
along the entire sequence

| Human | | | | | | |
|---|---|---|---|---|---|---|
| sequences | ABCA1 | ABCA4 | ABCA7 | ABCA5 | ABCA9 | ABCA12 |
| ABCA1 | 100/100 | | | | | |
| ABCA4 | 60/52 | 100/100 | | | | |
| ABCA7 | 63/54 | 58/49 | 100/100 | | | |
| ABCA5 | 41/31 | 41/30 | 40/29 | 100/100 | | |
| ABCA9 | 41/31 | 40/30 | 42/32 | 53/43 | 100/100 | |
| ABCA12 | 47/36 | 46/35 | 46/36 | 40/28 | 39/28 | 100/100 |
| form A | | | | | | |

Nucleotide Probes and Primers

Nucleotide probes and primers hybridizing with a nucleic acid (genomic DNA, messenger RNA, cDNA) according to the invention also form part of the invention.

According to the invention, nucleic acid fragments derived from a polynucleotide comprising any one of SEQ ID NOs: 1-4 or of a complementary nucleotide sequence are useful for the detection of the presence of at least one copy of a nucleotide sequence of the ABCA12 gene or of a fragment or of a variant (containing a mutation or a polymorphism) thereof in a sample.

The nucleotide probes or primers according to the invention comprise a nucleotide sequence comprising any one of SEQ ID NOs: 1-4, or a complementary nucleotide sequence.

The nucleotide probes or primers according to the invention comprise at least 8 consecutive nucleotides of a nucleic acid comprising any one of SEQ ID NOs: 1-4, or a complementary nucleotide sequence.

Preferably, nucleotide probes or primers according to the invention have a length of 10, 12, 15, 18 or 20 to 25, 35, 40, 50, 70, 80, 100, 200, 500, 1000, 1500 consecutive nucleotides of a nucleic acid according to the invention, in particular of a nucleic acid comprising any one of SEQ ID NOs: 1-4, or a complementary nucleotide sequence.

Alternatively, a nucleotide probe or primer according to the invention consists of and/or comprise the fragments having a length of 12, 15, 18, 20, 25, 35, 40, 50, 100, 200, 500, 1000, 1500 consecutive nucleotides of a nucleic acid according to the invention, more particularly of a nucleic acid comprising any one of SEQ ID NOs: 1-4, or a complementary nucleotide sequence.

The definition of a nucleotide probe or primer according to the invention therefore covers oligonucleotides which hybridize, under the high stringency hybridization conditions defined above, with a nucleic acid comprising any one of SEQ ID NOs: 1-4, or a complementary nucleotide sequence.

According to a preferred embodiment, a nucleotide primer according to the invention comprises a nucleotide sequence of any one of SEQ ID NOs: 7-38, or a complementary nucleic acid sequence.

Sequences of primers which make it possible to amplify various regions of the ABCA12 gene are presented in Table 3 below. The location of each primer of SEQ ID NOs: 7-38 within SEQ ID NOs: 1, and its hybridizing region is indicated in Table 3. The abbreviation “Comp” refers to the complementary nucleic acid sequence.

TABLE 3

Primers for the amplification of nucleic fragments
of the ABCA12 gene

| SEQ ID | | POSITION IN |
|---|---|---|
| NOs: | SEQUENCE (5′-3′) | SEQ ID NO 1 |
| 7 | GAAGAGTTGATTGAGAAGTGC | 1-21 |
| 8 | CGAAGAGAACTATGTGACAGC | 761-781 |
| 9 | CTTCTCACAAGTGCAAGAGC | 976-995 |
| 10 | CGCAATGGTTCCTATGAAGATTAC | 1451-1474 |
| 11 | CAGAAGGGTGAGTCCGATGAGGTAAGAC | comp 2116-2143 |
| 12 | GCTGTCACATAGTTCTCTTCG | comp 761-781 |
| 13 | GTAATCTTCATAGGAACCATTGCG | comp 1451-1474 |
| 14 | CCTACACACGGTACGGAAGAACATG | 4456-4480 |
| 15 | GCCATCGTCATAAGAGAGTTGGAACAC | 4629-4655 |
| 16 | GTGCTTATGGTTGCCTGGG | 3434-3451 |
| 17 | CTTCCATCTGTTAAACCAGG | 2776-2795 |
| 18 | GGTGTTCTGGCTGCATTC | 2014-2031 |
| 19 | GCCTCATCTACATCATTGCC | 3759-3778 |
| 20 | GTGTTCCAACTCTCTTATGACGATGGC | comp 4629-4655 |
| 21 | CATGTTCTTCCGTACCGTGTGTAGG | comp 4456-4480 |
| 22 | GGCAATGATGTAGATGAGGC | comp 3759-3778 |
| 23 | CCCAGGCAACCATAAGCAC | comp 3434-3452 |
| 24 | CTTTTCTACTGGCTTTTGATCTTTCCTCGG | 2215-2186 |
| 25 | CCTTGATAGGGAAACCTTC | 7428-7446 |
| 26 | CACCAGCATATACATTAGCA | comp 7115-7134 |
| 27 | GAAGGTTTCCCTATCAAGG | comp 7428-7446 |
| 28 | GTATCATGTACCAGTCACAGCAGGAGG | 7786-7812 |
| 29 | CCAAAGACCAGAAGTCCTATGAAACTGC | 7917-7944 |
| 30 | GAGTGGAGAAGAAAAGTCAG | 8363-8382 |
| 31 | CACGGAACCTAGATTCACTCC | 8652-8672 |
| 32 | CCCAGAGCAAGTGATTTC | comp 8712-8729 |
| 33 | CGAGTGCCCGTAGGAGTG | comp 5118-5135 |
| 34 | TTGCACCTAGTTTATTCATCTC | comp 6764-6785 |
| 35 | GTCATAAATGAAGTTTGTTACCC | comp 6312-6334 |
| 36 | CAACAGTTATCCAGAGATTCA | 5533-5553 |
| 37 | GAGTCCCTGCCAATAGAAC | 5970-5988 |
| 38 | GCAAATGCAGTATGTGACAC | 4976-4995 |

A nucleotide primer or probe according to the invention may be prepared by any suitable method well known to persons skilled in the art, including by cloning and action of restriction enzymes or by direct chemical synthesis according to techniques such as the phosophodiester method by Narang et al. (1979, Methods Enzymol, 68:90-98) or by Brown et al. (1979, Methods Enzymol, 68:109-151), the diethylphosphoramidite method by Beaucage et al. (1981, Tetrahedron Lett, 22: 1859-1862) or the technique on a solid support described in EU patent No. EP 0,707,592.

Each of the nucleic acids according to the invention, including the oligonucleotide probes and primers described above, may be labeled, if desired, by incorporating a marker which can be detected by spectroscopic, photochemical, biochemical, immunochemical or chemical means. For example, such markers may consist of radioactive isotopes (³²P, ³³P, ³H, 35S), fluorescent molecules (5-bromodeoxyuridine, fluorescein, acetylaminofluorene, digoxigenin) or ligands such as biotin. The labeling of the probes is preferably carried out by incorporating labeled molecules into the polynucleotides by primer extension, or alternatively by addition to the 5′ or 3′ ends. Examples of nonradioactive labeling of nucleic acid fragments are described in particular in French patent No. 78 109 75 or in the articles by Urdea et al. (1988, Nucleic Acids Research, 11:4937-4957) or Sanchez-pescador et al. (1988, J. Clin. Microbiol., 26(10):1934-1938).

Preferably, the nucleotide probes and primers according to the invention may have structural characteristics of the type to allow amplification of the signal, such as the probes described by Urdea et al. (1991, Nucleic Acids Symp Ser., 24:197-200) or alternatively in European patent No. EP-0,225,807 (CHIRON).

The oligonucleotide probes according to the invention may be used in particular in Southern-type hybridizations with the genomic DNA or alternatively in northern-type hybridizations with the corresponding messenger RNA when the expression of the corresponding transcript is sought in a sample.

The probes and primers according to the invention may also be used for the detection of products of PCR amplification or alternatively for the detection of mismatches.

Nucleotide probes or primers according to the invention may be immobilized on a solid support. Such solid supports are well known to persons skilled in the art and comprise surfaces of wells of microtiter plates, polystyrene beads, magnetic beads, nitrocellulose bands or microparticles such as latex particles.

Consequently, the present invention also relates to a method of detecting the presence of a nucleic acid comprising a nucleotide sequence of any one of SEQ ID NOs: 1-4, or of a complementary nucleotide sequence, or a nucleic acid fragment or variant of any one of SEQ ID NOs: 1-4, or of a complementary nucleotide sequence in a sample, said method comprising the steps of:

1) bringing one or more nucleotide probes or primers according to the invention into contact with the sample to be tested;

2) detecting the complex which may have formed between the probe(s) and the nucleic acid present in the sample.

According to a specific embodiment of the method of detection according to the invention, the oligonucleotide probes and primers are immobilized on a support.

According to another aspect, the oligonucleotide probes and primers comprise a detectable marker.

The invention relates, in addition, to a box or kit for detecting the presence of a nucleic acid according to the invention in a sample, said box or kit comprising:

a) one or more nucleotide probe(s) or primer(s) as described above;

b) where appropriate, the reagents necessary for the hybridization reaction.

According to a first aspect, the detection box or kit is characterized in that the probe(s) or primer(s) are immobilized on a support.

According to a second aspect, the detection box or kit is characterized in that the oligonucleotide probes comprise a detectable marker.

According to a specific embodiment of the detection kit described above, such a kit will comprise a plurality of oligonucleotide probes and/or primers in accordance with the invention which may be used to detect a target nucleic acid of interest or alternatively to detect mutations in the coding regions or the non-coding regions of the nucleic acids according to the invention, more particularly of nucleic acids comprising any one of SEQ ID NOs: 1-4, or a complementary nucleotide sequence.

Thus, the probes according to the invention, immobilized on a support, may be ordered into matrices such as “DNA chips”. Such ordered matrices have in particular been described in U.S. Pat. No. 5,143,854, in published PCT applications WO 90/15070 and WO 92/10092.

Support matrices on which oligonucleotide probes have been immobilized at a high density are for example described in U.S. Pat. No. 5,412,087 and in published PCT application WO 95/11995.

The nucleotide primers according to the invention may be used to amplify any one of the nucleic acids according to the invention, and more particularly a nucleic acid comprising a nucleotide sequence of any one of SEQ ID NOs: 1-4, or of a complementary nucleotide sequence. Alternatively, the nucleotide primers according to the invention may be used to amplify a nucleic acid fragment or variant of any one of SEQ ID NOs: 1-4, or of a complementary nucleotide sequence.

In a particular embodiment, the nucleotide primers according to the invention may be used to amplify a nucleic acid comprising any one of SEQ ID NOs: 1-4, or as depicted in any one of SEQ ID NOs: 1-4, or of a complementary nucleotide sequence.

Another subject of the invention relates to a method of amplifying a nucleic acid according to the invention, and more particularly a nucleic acid comprising a) any one of SEQ ID NOs: 1-4, or a complementary nucleotide sequence, b) as depicted in any one of SEQ ID NOs: 1-4, or of a complementary nucleotide sequence, contained in a sample, said method comprising the steps of:

a) bringing the sample in which the presence of the target nucleic acid is suspected into contact with a pair of nucleotide primers whose hybridization position is located respectively on the 5′ side and on the 3′ side of the region of the target nucleic acid whose amplification is sought, in the presence of the reagents necessary for the amplification reaction; and

b) detecting the amplified nucleic acids.

To carry out the amplification method as defined above, use will be preferably made of any of the nucleotide primers described above.

The subject of the invention is, in addition, a box or kit for amplifying a nucleic acid according to the invention, and more particularly a nucleic acid comprising any one of SEQ ID NOs: 1-4, or a complementary nucleotide sequence, or as depicted in any one of SEQ ID NOs: 1-4, or of a complementary nucleotide sequence, said box or kit comprising:

a) a pair of nucleotide primers in accordance with the invention, whose hybridization position is located respectively on the 5′ side and 3′ side of the target nucleic acid whose amplification is sought; and optionally,

b) reagents necessary for the amplification reaction.

Such an amplification box or kit will preferably comprise at least one pair of nucleotide primers as described above.

The subject of the invention is, in addition, a box or kit for amplifying all or part of a nucleic acid comprising any one of SEQ ID NOs: 1-4, or a complementary nucleotide sequence, said box or kit comprising:

1) a pair of nucleotide primers in accordance with the invention, whose hybridization position is located respectively on the 5′ side and 3′ side of the target nucleic acid whose amplification is sought; and optionally,

2) reagents necessary for an amplification reaction.

Such an amplification box or kit will preferably comprise at least one pair of nucleotide primers as described above.

The invention also relates to a box or kit for detecting the presence of a nucleic acid according to the invention in a sample, said box or kit comprising:

a) one or more nucleotide probes according to the invention;

b) where appropriate, reagents necessary for a hybridization reaction.

According to a first aspect, the detection box or kit is characterized in that the nucleotide probe(s) and primer(s)are immobilized on a support.

According to a second aspect, the detection box or kit is characterized in that the nucleotide probe(s) and primer(s) comprise a detectable marker.

According to a specific embodiment of the detection kit described above, such a kit will comprise a plurality of oligonucleotide probes and/or primers in accordance with the invention which may be used to detect target nucleic acids of interest or alternatively to detect mutations in the coding regions or the non-coding regions of the nucleic acids according to the invention. According to preferred embodiment of the invention, the target nucleic acid comprises a nucleotide sequence of any one of SEQ ID NOs: 1-4, or of a complementary nucleic acid sequence. Alternatively, the target nucleic acid is a nucleic acid fragment or variant of a nucleic acid comprising any one of SEQ ID NOs: 1-4, or of a complementary nucleotide sequence.

According to the present invention, a primer according to the invention comprises, generally, all or part of any one of SEQ ID NOs: 7-38, or a complementary sequence.

The nucleotide primers according to the invention are particularly useful in methods of genotyping subjects and/or of genotyping populations, in particular in the context of studies of association between particular allele forms or particular forms of groups of alleles (haplotypes) in subjects and the existence of a particular phenotype (character) in these subjects, for example the predisposition of these subjects to develop a pathology whose candidate chromosomal region is situated on chromosome 2, more precisely on the 2q arm and still more precisely in the 2q34 locus, such as the lamellar ichthyosis, the polymorphic congenital cataract, or the insulin dependant diabetes mellitus.

Recombinant Vectors

The invention also relates to a recombinant vector comprising a nucleic acid according to the invention. “Vector” for the purposes of the present invention will be understood to mean a circular or linear DNA or RNA molecule which is either in single-stranded or double-stranded form.

Preferably, such a recombinant vector will comprise a nucleic acid chosen from the following nucleic acids:

a) a nucleic acid comprising a nucleotide sequence of any one of SEQ ID NOs: 1-4, or of a complementary nucleotide sequence,

b) a nucleic acid comprising a nucleotide sequence as depicted in any one of SEQ ID NOs: 1-4, or of a complementary nucleotide sequence,

c) a nucleic acid having at least eight consecutive nucleotides of a nucleic acid comprising a nucleotide sequence of any one of SEQ ID NOs: 1-4, or of a complementary nucleotide sequence;

d) a nucleic acid having at least 80% nucleotide identity with a nucleic acid comprising a nucleotide sequence of any one of SEQ ID NOs: 1-4, or a complementary nucleotide sequence;

e) a nucleic acid having 85%, 90%, 95%, or 98% nucleotide identity with a nucleic acid comprising a nucleotide sequence of any one of SEQ ID NOs: 1-4, or a complementary nucleotide sequence;

f) a nucleic acid hybridizing, under high stringency hybridization conditions, with a nucleic acid comprising a nucleotide sequence of 1) any one of SEQ ID NOs: 1-4, or a complementary nucleotide sequence;

g) a nucleic acid encoding a polypeptide comprising an amino acid sequence of SEQ ID NO: 5 or 6; and

h) a nucleic acid encoding a polypeptide comprising amino acid sequence selected from SEQ ID NO: 5 or 6.

According to a first embodiment, a recombinant vector according to the invention is used to amplify a nucleic acid inserted therein, following transformation or transfection of a desired cellular host.

According to a second embodiment, a recombinant vector according to the invention corresponds to an expression vector comprising, in addition to a nucleic acid in accordance with the invention, a regulatory signal or nucleotide sequence that directs or controls transcription and/or translation of the nucleic acid and its encoded mRNA.

According to a preferred embodiment, a recombinant vector according to the invention will comprise in particular the following components:

(1) an element or signal for regulating the expression of the nucleic acid to be inserted, such as a promoter and/or enhancer sequence;

(2) a nucleotide coding region comprised within the nucleic acid in accordance with the invention to be inserted into such a vector, said coding region being placed in phase with the regulatory element or signal described in (1); and

(3) an appropriate nucleic acid for initiation and termination of transcription of the nucleotide coding region of the nucleic acid described in (2).

In addition, the recombinant vectors according to the invention may include one or more origins for replication in the cellular hosts in which their amplification or their expression is sought, markers or selectable markers.

By way of example, the bacterial promoters may be the LacI or LacZ promoters, the T3 or T7 bacteriophage RNA polymerase promoters, the lambda phage PR or PL promoters.

The promoters for eukaryotic cells will comprise the herpes simplex virus (HSV) virus thymidine kinase promoter or alternatively the mouse metallothionein-L promoter.

Generally, for the choice of a suitable promoter, persons skilled in the art can preferably refer to the book by Sambrook et al. (1989, Molecular cloning: a laboratory manual. 2ed. Cold Spring Harbor Laboratory, Cold spring Harbor, N.Y.) cited above or to the techniques described by Fuller et al. (1996, Immunology, In: Current Protocols in Molecular Biology, Ausubel et al.(eds.).

When the expression of the genomic sequence of the ABCA12 gene will be sought, use will preferably be made of the vectors capable of containing large insertion sequences. In a particular embodiment, bacteriophage vectors such as the P1 bacteriophage vectors such as the vector p158 or the vector p158/neo8 described by Sternberg (1992, Trends Genet., 8:1-16; 1994, Mamm. Genome, 5:397-404) will be preferably used.

The preferred bacterial vectors according to the invention are for example the vectors pBR322(ATCC37017) or alternatively vectors such as pAA223-3 (Pharmacia, Uppsala, Sweden), and pGEM1 (Promega Biotech, Madison, Wis., UNITED STATES).

There may also be cited other commercially available vectors such as the vectors pQE70, pQE60, pQE9 (Qiagen), psiX174, pBluescript SA, pNH8A, pNH16A, pNH18A, pNH46A, pWLNEO, pSV2CAT, pOG44, pXTI, pSG (Stratagene).

They may also be vectors of the baculovirus type such as the vector pVL1392/1393 (Pharmingen) used to transfect cells of the Sf9 line (ATCC No. CRL 1711) derived from Spodoptera frugiperda.

They may also be adenoviral vectors such as the human adenovirus of type 2 or 5.

A recombinant vector according to the invention may also be a retroviral vector or an adeno-associated vector (AAV). Such adeno-associated vectors are for example described by Flotte et al. (1992, Am. J. Respir. Cell Mol. Biol., 7:349-356), Samulski et al. (1989, J. Virol., 63:3822-3828), or McLaughlin B A et al. (1996, Am. J. Hum. Genet., 59:561-569).

To allow the expression of a polynucleotide according to the invention, the latter must be introduced into a host cell. The introduction of a polynucleotide according to the invention into a host cell may be carried out in vitro, according to the techniques well known to persons skilled in the art for transforming or transfecting cells, either in primer culture, or in the form of cell lines. It is also possible to carry out the introduction of a polynucleotide according to the invention in vivo or ex vivo, for the prevention or treatment of diseases linked to ABC A12 deficiencies.

To introduce a polynucleotide or vector of the invention into a host cell, a person skilled in the art can preferably refer to various techniques, such as the calcium phosphate precipitation technique (Graham et al., 1973, Virology, 52:456-457; Chen et al., 1987, Mol. Cell. Biol., 7 : 2745-2752), DEAE Dextran (Gopal, 1985, Mol. Cell. Biol., 5:1188-1190), electroporation (Tur-Kaspa, 1896, Mol. Cell. Biol., 6:716-718; Potter et al., 1984, Proc Natl Acad Sci U S A., 81(22):7161-5), direct microinjection (Harland et al., 1985, J. Cell. Biol., 101:1094-1095), liposomes charged with DNA (Nicolau et al., 1982, Methods Enzymol., 149:157-76; Fraley et al., 1979, Proc. Natl. Acad. Sci. USA, 76:3348-3352).

Once the polynucleotide has been introduced into the host cell, it may be stably integrated into the genome of the cell. The intregration may be achieved at a precise site of the genome, by homologous recombination, or it may be randomly integrated. In some embodiments, the polynucleotide may be stably maintained in the host cell in the form of an episome fragment, the episome comprising sequences allowing the retention and the replication of the latter, either independently, or in a synchronized manner with the cell cycle.

According to a specific embodiment, a method of introducing a polynucleotide according to the invention into a host cell, in particular a host cell obtained from a mammal, in vivo, comprises a step during which a preparation comprising a pharmaceutically compatible vector and a “naked” polynucleotide according to the invention, placed under the control of appropriate regulatory sequences, is introduced by local injection at the level of the chosen tissue, for example myocardial tissue, the “naked” polynucleotide being absorbed by the myocytes of this tissue.

Compositions for use in vitro and in vivo comprising “naked” polynucleotides are for example described in PCT Application No. WO 95/11307 (Institut Pasteur, Inserm, University of Ottawa) as well as in the articles by Tacson et al. (1996, Nature Medicine, 2(8):888-892) and Huygen et al. (1996, Nature Medicine, 2(8):893-898).

According to a specific embodiment of the invention, a composition is provided for the in vivo production of any one of ABCA12 proteins. This composition comprises a polynucleotide encoding the ABCA12 polypeptides placed under the control of appropriate regulatory sequences, in solution in a physiologically acceptable vector.

The quantity of vector which is injected into the host organism chosen varies according to the site of the injection. As a guide, there may be injected between about 0.1 and about 100 μg of polynucleotide encoding the ABCA12 proteins into the body of an animal, preferably into a patient likely to develop a disease linked with the ABCA12 gene deficiencies. Consequently, the invention also relates to a pharmaceutical composition intended for the prevention of or treatment of a patient or subject affected by ABCA12 deficiencies, comprising a nucleic acid encoding a short or full length ABCA12 protein, in combination with one or more physiologically compatible excipients.

Preferably, such a composition will comprise a nucleic acid comprising a nucleotide sequence of any one of SEQ ID NO: 1-4, wherein the nucleic acid is placed under the control of an appropriate regulatory element or signal.

The subject of the invention is, in addition, a pharmaceutical composition intended for the prevention of or treatment of a patient or a subject affected by ABCA12 deficiencies, comprising a recombinant vector according to the invention, in combination with one or more physiologically compatible excipients.

The invention also relates to the use of a nucleic acid according to the invention, encoding any one of the ABCA12 protein isoforms, for the manufacture of a medicament intended for the prevention or the treatment of subjects affected by a dysfunction of liphophilic substances transport or by a pathology located on the chromosome locus 2q34 such as for example the lamellar ichthyosis, the polymorphic congenital cataract, or insulin-dependant diabete mellitus.

The invention also relates to the use of a recombinant vector according to the invention, comprising a nucleic acid encoding any one of the ABCA12 proteins, for the manufacture of a medicament intended for the prevention or treatment of subjects affected by a dysfunction of liphophilic substances transport or by a pathology located on the chromosome locus 2q34 such as for example the lamellar ichthyosis, the polymorphic congenital cataract, or insulin-dependant diabete mellitus.

The subject of the invention is therefore also a recombinant vector comprising a nucleic acid according to the invention that encodes any one of ABCA12 proteins or polypeptides.

The invention also relates to the use of such a recombinant vector for the preparation of a pharmaceutical composition intended for the treatment and/or for the prevention of diseases or conditions associated with deficiency of transport of liphophilic substances transport or of pathology located on the chromosome locus 2q34 such as for example the lamellar ichthyosis, the polymorphic congenital cataract, or insulin-dependant diabete mellitus.

The present invention also relates to the use of cells genetically modified ex vivo with such a recombinant vector according to the invention, or of cells producing a recombinant vector, wherein the cells are implanted in the body, to allow a prolonged and effective expression in vivo of at least a biologically active ABCA12 polypeptide.

Vectors useful in methods of somatic gene therapy and compositions containing such vectors.

The present invention also relates to a new therapeutic approach for the treatment of pathologies linked to ABCA12 deficiencies. It provides an advantageous solution to the disadvantages of the prior art, by demonstrating the possibility of treating the pathologies ABCA12 deficiencies by gene therapy, by the transfer and expression in vivo of a gene encoding at least one of ABCA12 proteins involved in the transport of lipophilic substances or in pathology located on the chromosome locus ²q3⁴. The invention thus offers a simple means allowing a specific and effective treatment of related pathologies such as, for example, diabetes, arteriosclerosis, inflammation, cardiovascular diseases, metabolic diseases, lipophilic substances related pathologies, lamellar ichthyosis, and polymorphic congenital cataract.

Gene therapy consists in correcting a deficiency or an abnormality (mutation, aberrant expression and the like) and in bringing about the expression of a protein of therapeutic interest by introducing genetic information into the affected cell or organ. This genetic information may be introduced either ex vivo into a cell extracted from the organ, the modified cell then being reintroduced into the body, or directly in vivo into the appropriate tissue. In this second case, various techniques exist, among which various transfection techniques involving complexes of DNA and DEAE-dextran (Pagano et al. (1967. J. Virol., 1:891), of DNA and nuclear proteins (Kaneda et al., 1989,Science 243:375), of DNA and lipids (Felgner et al., 1987, PNAS 84:7413), the use of liposomes (Fraley et al., 1980, J.Biol.Chem., 255:10431), and the like. More recently, the use of viruses as vectors for the transfer of genes has appeared as a promising alternative to these physical transfection techniques. In this regard, various viruses have been tested for their capacity to infect certain cell populations. In particular, the retroviruses (RSV, HMS, MMS, and the like), the HSV virus, the adeno-associated viruses and the adenoviruses.

The present invention therefore also relates to a new therapeutic approach for the treatment of pathologies linked to ABCA12 deficiencies, consisting in transferring and expressing in vivo a gene encoding ABCA12. In a particularly preferred manner, the applicant has now found that it is possible to construct recombinant vectors comprising a nucleic acid encoding at least one ABCA12 protein isoform, to administer these recombinant vectors in vivo, and that this administration allows a stable and effective expression of at least one of the biologically active ABCA12 proteins in vivo, with no cytopathological effect.

Adenoviruses constitute particularly efficient vectors for the transfer and the expression of the ABCA12 gene. The use of recombinant adenoviruses as vectors makes it possible to obtain sufficiently high levels of expression of this gene to produce the desired therapeutic effect. Other viral vectors such as retroviruses or adeno-associated viruses (AAV) can allow a stable expression of the gene are also claimed.

The present invention is thus likely to offer a new approach for the treatment and prevention of ABCA12 deficiencies.

The subject of the invention is therefore also a defective recombinant virus comprising a nucleic acid according to the invention that encodes at least one ABCA12 protein isoform involved in the metabolism of lipophilic substances or in pathology located on the chromosome locus 2q34 such as for example the lamellar ichthyosis, the polymorphic congenital cataract, or insulin-dependant diabete mellitus.

The invention also relates to the use of such a defective recombinant virus for the preparation of a pharmaceutical composition which may be useful for the treatment and/or for the prevention of ABCA12 deficiencies.

The present invention also relates to the use of cells genetically modified ex vivo with such a defective recombinant virus according to the invention, or of cells producing a defective recombinant virus, wherein the cells are implanted in the body, to allow a prolonged and effective expression in vivo of at least one biologically active ABCA12 polypeptides.

The present invention is particularly advantageous because it makes it possible to induce a controlled expression, and with no harmful effect, of ABCA12 in organs which are not normally involved in the expression of this protein. In particular, a significant release of the short or full length ABCA12 protein is obtained by implantation of cells producing vectors of the invention, or infected ex vivo with vectors of the invention.

The activity of these ABC protein transporters produced in the context of the present invention may be of the human or animal ABCA12 type. The nucleic sequence used in the context of the present invention may be a cDNA, a genomic DNA (gDNA), an RNA (in the case of retroviruses) or a hybrid construct consisting, for example, of a cDNA into which one or more introns (gDNA) would be inserted. It may also involve synthetic or semisynthetic sequences. In a particularly advantageous manner, a cDNA or a gDNA is used. In particular, the use of a gDNA allows a better expression in human cells. To allow their incorporation into a viral vector according to the invention, these sequences are preferably modified, for example by site-directed mutagenesis, in particular for the insertion of appropriate restriction sites. The sequences described in the prior art are indeed not constructed for use according to the invention, and prior adaptations may prove necessary, in order to obtain substantial expressions. In the context of the present invention, the use of a nucleic sequence encoding any one of human ABCA12 proteins is preferred. Moreover, it is also possible to use a construct encoding a derivative of any one of ABCA12 proteins. A derivative of any one of ABCA12 proteins comprises, for example, any sequence obtained by mutation, deletion and/or addition relative to the native sequence. These modifications may be made by techniques known to a person skilled in the art (see general molecular biological techniques below). The biological activity of the derivatives thus obtained can then be easily determined, as indicated in particular in the examples of the measurement of the efflux of the substrate from cells. The derivatives for the purposes of the invention may also be obtained by hybridization from nucleic acid libraries, using as probe the native sequence or a fragment thereof.

These derivatives are in particular molecules having a higher affinity for their binding sites, molecules exhibiting greater resistance to proteases, molecules having a higher therapeutic efficacy or fewer side effects, or optionally new biological properties. The derivatives also include the modified DNA sequences allowing improved expression in vivo.

In a first embodiment, the present invention relates to a defective recombinant virus comprising a cDNA encoding a short or full length ABCA12 polypeptide. In another preferred embodiment of the invention, a defective recombinant virus comprises a genomic DNA (gDNA) encoding any one of the ABCA12 polypeptides isoforms. Preferably, the ABCA12 polypeptides comprise an amino acid sequence selected from SEQ ID NO:5 or 6, respectively.

The vectors of the invention may be prepared from various types of viruses. Preferably, vectors derived from adenoviruses, adeno-associated viruses (AAV), herpesviruses (HSV) or retroviruses are used. It is preferable to use an adenovirus, for direct administration or for the ex vivo modification of cells intended to be implanted, or a retrovirus, for the implantation of producing cells.

The viruses according to the invention are defective, that is to say that they are incapable of autonomously replicating in the target cell. Generally, the genome of the defective viruses used in the context of the present invention therefore lacks at least the sequences necessary for the replication of said virus in the infected cell. These regions may be either eliminated (completely or partially), or made non functional, or substituted with other sequences and in particular with the nucleic sequence encoding any one of the ABCA12 proteins. Preferably, the defective virus retains, nevertheless, the sequences of its genome which are necessary for the encapsidation of the viral particles.

As regards more particularly adenoviruses, various serotypes, whose structure and properties vary somewhat, have been characterized. Among these serotypes, human adenoviruses of type 2 or 5 (Ad 2 or Ad 5) or adenoviruses of animal origin (see Application WO 94/26914) are preferably used in the context of the present invention. Among the adenoviruses of animal origin which can be used in the context of the present invention, there may be mentioned adenoviruses of canine, bovine, murine (example: Mav1, Beard et al., Virology 75 (1990) 81), ovine, porcine, avian or simian (example: SAV) origin. Preferably, the adenovirus of animal origin is a canine adenovirus, more preferably a CAV2 adenovirus [Manhattan or A26/61 strain (ATCC VR-800) for example]. Preferably, adenoviruses of human or canine or mixed origin are used in the context of the invention. Preferably, the defective adenoviruses of the invention comprise the ITRs, a sequence allowing the encapsidation and the sequence encoding any one of the ABCA12 proteins. Preferably, in the genome of the adenoviruses of the invention, the E1 region at least is made non functional. Still more preferably, in the genome of the adenoviruses of the invention, the E1 gene and at least one of the E2, E4 and L1-L5 genes are non functional. The viral gene considered may be made non functional by any technique known to a person skilled in the art, and in particular by total suppression, by substitution, by partial deletion or by addition of one or more bases in the gene(s) considered. Such modifications may be obtained in vitro (on the isolated DNA) or in situ, for example, by means of genetic engineering techniques, or by treatment by means of mutagenic agents. Other regions may also be modified, and in particular the E3 (WO95/02697), E2 (WO94/28938), E4 (WO94/28152, WO94/12649, WO95/02697) and L5 (WO95/02697) region. According to a preferred embodiment, the adenovirus according to the invention comprises a deletion in the E1 and E4 regions and the sequence encoding any one of ABCA12 is inserted at the level of the inactivated E1 region. According to another preferred embodiment, it comprises a deletion in the E1 region at the level of which the E4 region and the sequence encoding any one of ABCA12 (French Patent Application FR94 13355) are inserted.

The defective recombinant adenoviruses according to the invention may be prepared by any technique known to persons skilled in the art (Levrero et al., 1991 Gene 101; EP 185 573; and Graham, 1984, EMBO J., 3:2917). In particular, they may be prepared by homologous recombination between an adenovirus and a plasmid carrying, inter alia, the nucleic acid encoding the short or full length ABCA12 protein. The homologous recombination occurs after cotransfection of said adenoviruses and plasmid into an appropriate cell line. The cell line used must preferably (i) be transformable by said elements, and (ii), contain the sequences capable of complementing the part of the defective adenovirus genome, preferably in integrated form in order to avoid the risks of recombination. By way of example of a line, there may be mentioned the human embryonic kidney line 293 (Graham et al., 1977, J. Gen. Virol., 36:59), which contains in particular, integrated into its genome, the left part of the genome of an AdS adenovirus (12%) or lines capable of complementing the E1 and E4 functions as described in particular in Applications WO 94/26914 and WO95/02697.

As regards the adeno-associated viruses (AAV), they are DNA viruses of a relatively small size, which integrate into the genome of the cells which they infect, in a stable and site-specific manner. They are capable of infecting a broad spectrum of cells, without inducing any effect on cellular growth, morphology or differentiation. Moreover, they do not appear to be involved in pathologies in humans. The genome of AAVs has been cloned, sequenced and characterized. It comprises about 4700 bases, and contains at each end an inverted repeat region (ITR) of about 145 bases, serving as replication origin for the virus. The remainder of the genome is divided into 2 essential regions carrying the encapsidation functions: the left hand part of the genome, which contains the rep gene, involved in the viral replication and the expression of the viral genes; the right hand part of the genome, which contains the cap gene encoding the virus capsid proteins.

The use of vectors derived from AAVs for the transfer of genes in vitro and in vivo has been described in the literature (see in particular WO 91/18088; WO 93/09239; U.S. Pat. Nos. 4,797,368, 5,139,941, EP 488 528). These applications describe various constructs derived from AAVs, in which the rep and/or cap genes are deleted and replaced by a gene of interest, and their use for transferring in vitro (on cells in culture) or in vivo (directly into an organism) said gene of interest. However, none of these documents either describes or suggests the use of a recombinant AAV for the transfer and expression in vivo or ex vivo of any one of ABCA12 proteins, or the advantages of such a transfer. The defective recombinant AAVs according to the invention may be prepared by cotransfection, into a cell line infected with a human helper virus (for example an adenovirus), of a plasmid containing the sequence encoding the short or full length ABCA12 protein bordered by two AAV inverted repeat regions (ITR), and of a plasmid carrying the AAV encapsidation genes (rep and cap genes). The recombinant AAVs produced are then purified by conventional techniques.

As regards the herpesviruses and the retroviruses, the construction of recombinant vectors has been widely described in the literature: see in particular Breakfield et al., (1991.New Biologist, 3:203); EP 453242, EP178220, Bernstein et al. (1985); McCormick, (1985. BioTechnology, 3:689), and the like.

In particular, the retroviruses are integrating viruses, infecting dividing cells. The genome of the retroviruses essentially comprises two long terminal repeats (LTRs), an encapsidation sequence and three coding regions (gag, pol and env). In the recombinant vectors derived from retroviruses, the gag, pol and env genes are generally deleted, completely or partially, and replaced with a heterologous nucleic acid sequence of interest. These vectors may be produced from various types of retroviruses such as in particular MoMuLV (“Murine Moloney Leukemia virus”; also called MoMLV), MSV (“murine moloney sarcoma virus”), HaSV (“Harvey Sarcoma virus”); SNV (“spleen necrosis virus”); RSV (“rous sarcoma virus”) or Friend's virus.

To construct recombinant retroviruses containing a sequence encoding any one of the ABCA12 proteins according to the invention, a plasmid containing in particular the LTRs, the encapsidation sequence and said coding sequence is generally constructed, and then used to transfect a so-called encapsidation cell line, capable of providing in trans the retroviral functions deficient in the plasmid. Generally, the encapsidation lines are therefore capable of expressing the gag, pol and env genes. Such encapsidation lines have been described in the prior art, and in particular the PA317 line (U.S. Pat. No. 4,861,719), the PsiCRIP line (WO 90 /02806) and the GP+envAm−12 line (WO 89/07150). Moreover, the recombinant retroviruses may contain modifications at the level of the LTRs in order to suppress the transcriptional activity, as well as extended encapsidation sequences, containing a portion of the gag gene (Bender et al., 1987, J. Virol., 61:1639). The recombinant retroviruses produced are then purified by conventional techniques.

To carry out the present invention, it is preferable to use a defective recombinant adenovirus. The particularly advantageous properties of adenoviruses are preferred for the in vivo expression of a protein having a lipophilic subtrate transport activity. The adenoviral vectors according to the invention are particularly preferred for a direct administration in vivo of a purified suspension, or for the ex vivo transformation of cells, in particular autologous cells, in view of their implantation. Furthermore, the adenoviral vectors according to the invention exhibit, in addition, considerable advantages, such as in particular their very high infection efficiency, which makes it possible to carry out infections using small volumes of viral suspension.

According to another particularly preferred embodiment of the invention, a line producing retroviral vectors containing the sequence encoding any one of the ABCA12 proteins is used for implantation in vivo. The lines which can be used to this end are in particular the PA317 (U.S. Pat. No. 4,861,719), PsiCrip (WO 90/02806) and GP+envAm−12 (U.S. Pat. No. 5,278,056) cells modified so as to allow the production of a retrovirus containing a nucleic sequence encoding the short or full length ABCA12 protein according to the invention. For example, totipotent stem cells, precursors of blood cell lines, may be collected and isolated from a subject. These cells, when cultured, may then be transfected with the retroviral vector containing the sequence encoding the short or full length ABCA12 protein under the control of viral, nonviral or nonviral promoters specific for macrophages or under the control of its own promoter. These cells are then reintroduced into the subject. The differentiation of these cells will be responsible for blood cells expressing at least one of ABCA12 proteins.

Preferably, in the vectors of the invention, the sequence encoding any one of the ABCA12 proteins is placed under the control of signals allowing its expression in the infected cells. These may be expression signals which are homologous or heterologous, that is to say signals different from those which are naturally responsible for the expression of the ABCA12 proteins. They may also be in particular sequences responsible for the expression of other proteins, or synthetic sequences. In particular, they may be sequences of eukaryotic or viral genes or derived sequences, stimulating or repressing the transcription of a gene in a specific manner or otherwise and in an inducible manner or otherwise. By way of example, they may be promoter sequences derived from the genome of the cell which it is desired to infect, or from the genome of a virus, and in particular the promoters of the E1A or major late promoter (MLP) genes of adenoviruses, the cytomegalovirus (CMV) promoter, the RSV-LTR and the like. Among the eukaryotic promoters, there may also be mentioned the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), tissue-specific promoters (pyruvate kinase, villin, promoter of the fatty acid binding intestinal protein, promoter of the smooth muscle cell α-actin, promoters specific for the liver; Apo AI, Apo AII, human albumin and the like) or promoters corresponding to a stimulus (steroid hormone receptor, retinoic acid receptor and the like). In addition, these expression sequences may be modified by addition of enhancer or regulatory sequences and the like. Moreover, when the inserted gene does not contain expression sequences, it may be inserted into the genome of the defective virus downstream of such a sequence.

In a specific embodiment, the invention relates to a defective recombinant virus comprising a nucleic acid encoding any one of ABCA12 proteins the control of a promoter chosen from RSV-LTR or the CMV early promoter.

As indicated above, the present invention also relates to any use of a virus as described above for the preparation of a pharmaceutical composition for the treatment and/or prevention of pathologies linked to the transport of lipophilic substances or located on the chromosome locus 2q34 such as for example the lamellar ichthyosis, the polymorphic congenital cataract, or insulin-dependant diabete mellitus.

The present invention also relates to a pharmaceutical composition comprising one or more defective recombinant viruses as described above. These pharmaceutical compositions may be formulated for administration by the topical, oral, parenteral, intranasal, intravenous, intramuscular, subcutaneous, intraocular or transdermal route and the like. Preferably, the pharmaceutical compositions of the invention comprises a pharmaceutically acceptable vehicle or physiologically compatible excipient for an injectable formulation, in particular for an intravenous injection, such as for example into the patient's portal vein. These may relate in particular to isotonic sterile solutions or dry, in particular, freeze-dried, compositions which, upon addition depending on the case of sterilized water or physiological saline, allow the preparation of injectable solutions. Direct injection into the patient's portal vein is preferred because it makes it possible to target the infection at the level of the liver and thus to concentrate the therapeutic effect at the level of this organ.

The doses of defective recombinant virus used for the injection may be adjusted as a function of various parameters, and in particular as a function of the viral vector, of the mode of administration used, of the relevant pathology or of the desired duration of treatment. In general, the recombinant adenoviruses according to the invention are formulated and administered in the form of doses of between 10⁴ and 10¹⁴ pfu/ml, and preferably 10⁶ to 10¹⁰ pfu/ml. The term “pfu” (plaque forming unit) corresponds to the infectivity of a virus solution, and is determined by infecting an appropriate cell culture and measuring, generally after 48 hours, the number of plaques that result from infected cell lysis. The techniques for determining the pfu titer of a viral solution are well documented in the literature.

As regards retroviruses, the compositions according to the invention may directly contain the producing cells, with a view to their implantation.

In this regard, another subject of the invention relates to any mammalian cell infected with one or more defective recombinant viruses according to the invention. More particularly, the invention relates to any population of human cells infected with such viruses. These may be in particular cells of blood origin (totipotent stem cells or precursors), fibroblasts, myoblasts, hepatocytes, keratinocytes, smooth muscle and endothelial cells, glial cells and the like.

The cells according to the invention may be derived from primary cultures. These may be collected by any technique known to persons skilled in the art and then cultured under conditions allowing their proliferation. As regards more particularly fibroblasts, these may be easily obtained from biopsies, for example according to the technique described by Ham (1980). These cells may be used directly for infection with the viruses, or stored, for example by freezing, for the establishment of autologous libraries, in view of a subsequent use. The cells according to the invention may be secondary cultures, obtained for example from pre-established libraries (see for example EP 228458, EP 289034, EP 400047, EP 456640).

The cells in culture are then infected with a recombinant virus according to the invention, in order to confer on them the capacity to produce at least one biologically active ABCA12 protein. The infection is carried out in vitro according to techniques known to persons skilled in the art. In particular, depending on the type of cells used and the desired number of copies of virus per cell, persons skilled in the art can adjust the multiplicity of infection and optionally the number of infectious cycles produced. It is clearly understood that these steps must be carried out under appropriate conditions of sterility when the cells are intended for administration in vivo. The doses of recombinant virus used for the infection of the cells may be adjusted by persons skilled in the art according to the desired aim. The conditions described above for the administration in vivo may be applied to the infection in vitro. For the infection with a retrovirus, it is also possible to co-culture a cell to be infected with a cell producing the recombinant retrovirus according to the invention. This makes it possible to eliminate purification of the retrovirus.

Another subject of the invention relates to an implant comprising mammalian cells infected with one or more defective recombinant viruses according to the invention or cells producing recombinant viruses, and an extracellular matrix. Preferably, the implants according to the invention comprise 10⁵ to 10¹⁰ cells. More preferably, they comprise 10⁶ to 10⁸ cells.

More particularly, in the implants of the invention, the extracellular matrix comprises a gelling compound and optionally a support allowing the anchorage of the cells.

For the preparation of the implants according to the invention, various types of gelling agents may be used. The gelling agents are used for the inclusion of the cells in a matrix having the constitution of a gel, and for promoting the anchorage of the cells on the support, where appropriate. Various cell adhesion agents can therefore be used as gelling agents, such as in particular collagen, gelatin, glycosaminoglycans, fibronectin, lectins and the like. Preferably, collagen is used in the context of the present invention. This may be collagen of human, bovine or murine origin. More preferably, type I collagen is used.

As indicated above, the compositions according to the invention preferably comprise a support allowing the anchorage of the cells. The term anchorage designates any form of biological and/or chemical and/or physical interaction causing the adhesion and/or the attachment of the cells to the support. Moreover, the cells may either cover the support used, or penetrate inside this support, or both. It is preferable to use in the context of the invention a solid, nontoxic and/or biocompatible support. In particular, it is possible to use polytetrafluoroethylene (PTFE) fibers or a support of biological origin.

The present invention thus offers a very effective means for the treatment or prevention of pathologies which are statistically linked with the locus 2q34 such as lamellar ichthyosis, polymorphic congenital cataract, and insulin dependant diabetes mellitus (IDDM13).

In addition, this treatment may be applied to both humans and any animals such as ovines, bovines, domestic animals (dogs, cats and the like), horses, fish and the like.

Recombinant Host Cells

The invention relates to a recombinant host cell comprising a nucleic acid of the invention, and more particularly, a nucleic acid comprising a nucleotide sequence selected from SEQ ID NO: 1-4, or a complementary nucleotide sequence thereof.

The invention also relates to a recombinant host cell comprising a nucleic acid of the invention, and more particularly a nucleic acid comprising a nucleotide sequence as depicted in SEQ ID NO: 1-4, or a complementary nucleotide sequence thereof.

According to another aspect, the invention also relates to a recombinant host cell comprising a recombinant vector according to the invention. Therefore, the invention also relates to a recombinant host cell comprising a recombinant vector comprising any of the nucleic acids of the invention, and more particularly a nucleic acid comprising a nucleotide sequence of selected from SEQ ID NO: 1-4, or a complementary nucleotide sequence thereof.

The invention also relates to a recombinant host cell comprising a recombinant vector comprising a nucleic acid comprising a nucleotide sequence as depicted in any one of SEQ ID NOs: 1-4, or of a complementary nucleotide sequence thereof.

The preferred host cells according to the invention are for example the following:

a) prokaryotic host cells: strains of Escherichia coli (strain DH5-α), of Bacillus subtilis, of Salmonella typhimurium, or strains of genera such as Pseudomonas, Streptomyces and Staphylococus;

b) eukaryotic host cells: HeLa cells (ATCC No. CCL2), Cv 1 cells (ATCC No. CCL70), COS cells (ATCC No. CRL 1650), Sf-9 cells (ATCC No. CRL 1711), CHO cells (ATCC No. CCL-61) or 3T3 cells (ATCC No. CRL-6361).

Methods for Producing ABCA12 Polypeptides

The invention also relates to a method for the production of a polypeptide comprising an amino acid sequence of any one of SEQ ID NOs: 5 or 6, said method comprising the steps of:

a) inserting a nucleic acid encoding said polypeptide into an appropriate vector;

b) culturing, in an appropriate culture medium, a previously transformed host cell or transfecting a host cell with the recombinant vector of step a);

c) recovering the conditioned culture medium or lysing the host cell, for example by sonication or by osmotic shock;

d) separating and purifying said polypeptide from said culture medium or alternatively from the cell lysates obtained in step c); and

e) where appropriate, characterizing the recombinant polypeptide produced.

The polypeptides according to the invention may be characterized by binding to an immunoaffinity chromatography column on which the antibodies directed against this polypeptide or against a fragment or a variant thereof have been previously immobilized.

According to another aspect, a recombinant polypeptide according to the invention may be purified by passing it over an appropriate series of chromatography columns, according to methods known to persons skilled in the art and described for example in F. Ausubel et al (1989, Current Protocols in Molecular Biology, Green Publishing Associates and Wiley Interscience, N.Y).

A polypeptide according to the invention may also be prepared by conventional chemical synthesis techniques either in homogeneous solution or in solid phase. By way of illustration, a polypeptide according to the invention may be prepared by the technique either in homogeneous solution described by Houben Weyl (1974, Meuthode der Organischen Chemie, E. Wunsch Ed., 15-I:15-II) or the solid phase synthesis technique described by Merrifield (1965, Nature, 207(996):522-523; 1965, Science, 150(693):178-185).

A polypeptide termed “homologous” to a polypeptide having an amino acid sequence selected from SEQ ID NO: 5 or 6 also forms part of the invention. Such a homologous polypeptide comprises an amino acid sequence possessing one or more substitutions of an amino acid by an equivalent amino acid of SEQ ID NO:5 or 6.

An “equivalent amino acid” according to the present invention will be understood to mean for example replacement of a residue in the L form by a residue in the D form or the replacement of a glutamic acid (E) by a pyro-glutamic acid according to techniques well known to persons skilled in the art. By way of illustration, the synthesis of peptide containing at least one residue in the D form is described by Koch (1977). According to another aspect, two amino acids belonging to the same class, that is to say two uncharged polar, nonpolar, basic or acidic amino acids, are also considered as equivalent amino acids.

Polypeptides comprising at least one nonpeptide bond such as a retro-inverse bond (NHCO), a carba bond (CH₂CH₂) or a ketomethylene bond (CO-CH₂) also form part of the invention.

Preferably, the polypeptides according to the invention comprising one or more additions, deletions, substitutions of at least one amino acid will retain their capacity to be recognized by antibodies directed against the nonmodified polypeptides.

Antibodies

The ABCA12 polypeptides according to the invention, in particular 1) a polypeptide comprising an amino acid sequence of any one of SEQ ID NOs: 5 or 6, 2) a polypeptide fragment or variant of a polypeptide comprising an amino acid sequence of any one of SEQ ID NOs: 5 or 6, or 3) a polypeptide termed “homologous” to a polypeptide comprising amino acid sequence selected from SEQ ID NOs: 5 or 6, may be used for the preparation of an antibody, in particular for detecting the production of a normal or altered form of ABCA12 polypeptides in a patient.

An antibody directed against a polypeptide termed “homologous” to a polypeptide having an amino acid sequence selected from SEQ ID NO: 5 or 6 also forms part of the invention. Such an antibody is directed against a homologous polypeptide comprising an amino acid sequence possessing one or more substitutions of an amino acid by an equivalent amino acid of SEQ ID NO: 5 or 6.

“Antibody” for the purposes of the present invention will be understood to mean in particular polyclonal or monoclonal antibodies or fragments (for example the F(ab)′₂ and Fab fragments) or any polypeptide comprising a domain of the initial antibody recognizing the target polypeptide or polypeptide fragment according to the invention.

Monoclonal antibodies may be prepared from hybridomas according to the technique described by Kohler and Milstein (1975, Nature, 256:495-497).

According to the invention, a polypeptide produced recombinantly or by chemical synthesis, and fragments or other derivatives or analogs thereof, including fusion proteins, may be used as an immunogen to generate antibodies that recognize a polypeptide according to the invention. Such antibodies include but are not limited to polyclonal, monoclonal, chimeric, single chain, Fab fragments, and an Fab expression library. The anti-ABCA12 antibodies of the invention may be cross reactive, e.g., they may recognize corresponding ABCA12 polypeptides from different species. Polyclonal antibodies have greater likelihood of cross reactivity. Alternatively, an antibody of the invention may be specific for a single form of any one of ABCA12. Preferably, such an antibody is specific for any one of human ABCA12 polypeptide isoforms.

Various procedures known in the art may be used for the production of polyclonal antibodies to any one of ABCA12 polypeptides, derivatives or analogs thereof. For the production of antibody, various host animals can be immunized by injection with the short or full length ABCA12 polypeptide, or a derivative (e.g., fragment or fusion protein) thereof, including but not limited to rabbits, mice, rats, sheep, goats, etc. In one embodiment, the short or full length ABCA12 polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward any one of ABCA12 polypeptides, or fragments, analogs, or derivatives thereof, any technique that provides for the production of antibody molecules by continuous cell lines in culture may be used. These include but are not limited to the hybridoma technique originally developed by Kohler and Milstein (1975, Nature, 256:495-497), as well as the trioma technique, the human B-cell hybridoma technique (Kozbor et al., 1983, Immunology Today, 4:72; Cote et al. 1983, Proc. Natl. Acad. Sci. U.S.A. 80:2026-2030), and the EBV-hybridoma technique to produce human monoclonal antibodies (Cole et al., 1985, In: Monoclonal Antibodies and Cancer Therapy, Alan R. Liss, Inc., pp. 77-96). In an additional embodiment of the invention, monoclonal antibodies can be produced in germ-free animals (WO 89/12690). In fact, according to the invention, techniques developed for the production of “chimeric antibodies” (Morrison et al., 1984, J. Bacteriol. 159:870; Neuberger et al., 1984, Nature, 312:604-608; Takeda et al., 1985, Nature, 314:452-454) by splicing the genes from a mouse antibody molecule specific for any one of ABCA12 polypeptides together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies (U.S. Pat. Nos. 5,476,786 and 5,132,405 to Huston; U.S. Pat. No. 4,946,778) can be adapted to produce ABCA12 polypeptide-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for any one of ABCA12 polypeptides, or its derivatives, or analogs.

Antibody fragments which contain the idiotype of the antibody molecule can be generated by known techniques. For example, such fragments include but are not limited to the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are within the scope of the present invention. For example, to select antibodies which recognize a specific epitope of any one of ABCA12 polypeptides, one may assay generated hybridomas for a product which binds to any one of ABCA12 polypeptide fragments containing such epitope. For selection of an antibody specific to any one of of ABCA12 polypeptides from a particular species of animal, one can select on the basis of positive binding with any one of ABCA12 polypeptides expressed by or isolated from cells of that species of animal.

The foregoing antibodies can be used in methods known in the art relating to the localization and activity of any one of ABCA12 polypeptides, e.g., for Western blotting, ABCA12 polypeptides in situ, measuring levels thereof in appropriate physiological samples, etc. using any of the detection techniques mentioned above or known in the art.

In a specific embodiment, antibodies that agonize or antagonize the activity of any one of ABCA12 polypeptides can be generated. Such antibodies can be tested using the assays described infra for identifying ligands.

The present invention relates to an antibody directed against 1) a polypeptide comprising an amino acid sequence of any one of SEQ ID NOs: 5 or 6, 2) a polypeptide fragment or variant of a polypeptide comprising an amino acid sequence of any one of SEQ ID NOs: 5 or 6, or 3) a polypeptide termed “homologous” to a polypeptide comprising amino acid sequence selected from SEQ ID NO:5 or 6, also forms part of the invention, as produced in the trioma technique or the hybridoma technique described by Kozbor et al. (1983, Hybridoma, 2(1):7-16).

The invention also relates to single-chain Fv antibody fragments (ScFv) as described in U.S. Pat. No. 4,946,778 or by Martineau et al. (1998, J Mol Biol, 280(1):117-127).

The antibodies according to the invention also comprise antibody fragments obtained with the aid of phage libraries as described by Ridder et al., (1995, Biotechnology (NY), 13(3):255-260) or humanized antibodies as described by Reinmann et al. (1997, AIDS Res Hum Retroviruses, 13(11):933-943) and Leger et al., (1997, Hum Antibodies, 8(1):3-16).

The antibody preparations according to the invention are useful in immunological detection tests intended for the identification of the presence and/or of the quantity of antigens present in a sample.

An antibody according to the invention may comprise, in addition, a detectable marker which is isotopic or nonisotopic, for example fluorescent, or may be coupled to a molecule such as biotin, according to techniques well known to persons skilled in the art.

Thus, the subject of the invention is, in addition, a method of detecting the presence of a polypeptide according to the invention in a sample, said method comprising the steps of:

a) bringing the sample to be tested into contact with an antibody directed against 1) a polypeptide comprising an amino acid sequence of any one of SEQ ID NOs: 5 or 6, 2) a polypeptide fragment or variant of a polypeptide comprising an amino acid sequence of any one of SEQ ID NOs: 5 or 6, or 3) a polypeptide termed “homologous” to a polypeptide comprising amino acid sequence selected from SEQ ID NOs: 5 or 6, and

b) detecting the antigen/antibody complex formed.

The invention also relates to a box or kit for diagnosis or for detecting the presence of a polypeptide in accordance with the invention in a sample, said box comprising:

a) an antibody directed against 1) a polypeptide comprising an amino acid sequence of any one of SEQ ID NOs:5 or 6, 2) a polypeptide fragment or variant of a polypeptide comprising an amino acid sequence of any one of SEQ ID NOs: 5 or 6, or 3) a polypeptide termed “homologous” to a polypeptide comprising amino acid sequence selected from SEQ ID NOs: 5 or 6, and

b) a reagent allowing the detection of the antigen/antibody complexes formed.

Pharmaceutical Compositions and Therapeutic Methods of Treatment

The invention also relates to pharmaceutical compositions intended for the prevention and/or treatment of pathology, characterized in that they comprise a therapeutically effective quantity of a polynucleotide capable of giving rise to the production of an effective quantity of at least one of ABCA12 functional polypeptides, in particular a polypeptide comprising an amino acid sequence of SEQ ID NOs: 5 or 6.

The invention also provides pharmaceutical compositions comprising a nucleic acid encoding any one of ABCA12 polypeptides according to the invention and pharmaceutical compositions comprising any one of ABCA12 polypeptides according to the invention intended for the prevention and/or treatment of diseases linked to a deficiency of the ABCA12 gene.

The present invention also relates to a new therapeutic approach for the treatment of pathologies linked to the deficiencies of ABCA12 gene.

Also, the present invention offers a new approach for the treatment and/or the prevention of pathologies linked to the abnormalities of the transport of lipophilic substances or located on the chromosome locus 2q34 such as for example the lamellar ichthyosis, the polymorphic congenital cataract, or insulin-dependant diabete mellitus.

Consequently, the invention also relates to a pharmaceutical composition intended for the prevention of or treatment of subjects affected by a dysfunction of the ABCA12 protein, comprising a nucleic acid encoding at least one ABCA12 protein, in combination with one or more physiologically compatible vehicle and/or excipient.

According to a specific embodiment of the invention, a composition is provided for the in vivo production of at least one of the ABCA12 proteins. This composition comprises a nucleic acid encoding any one of the ABCA12 polypeptides placed under the control of appropriate regulatory sequences, in solution in a physiologically acceptable vehicle and/or excipient.

Therefore, the present invention also relates to a composition comprising a nucleic acid encoding a polypeptide comprising an amino acid sequence of SEQ ID NOs: 5 or 6, wherein the nucleic acid is placed under the control of appropriate regulatory elements.

Preferably, such a composition will comprise a nucleic acid comprising a nucleotide sequence of SEQ ID NOs: 1-4, placed under the control of appropriate regulatory elements.

According to another aspect, the subject of the invention is also a preventive and/or curative therapeutic method of treating diseases caused by a deficiency of the ABCA12 gene, such a method comprising a step in which there is administration to a patient of nucleic acid encoding any one of the ABCA12 polypeptides according to the invention in said patient, said nucleic acid being, where appropriate, combined with one or more physiologically compatible vehicles and/or excipients.

The invention also relates to a pharmaceutical composition intended for the prevention of or treatment of subjects affected by a dysfunction in the transport of lipophilic substances or by a pathology located on the chromosome locus 2q34 such as for example the lamellar ichthyosis, the polymorphic congenital cataract, or insulin-dependant diabete mellitus, comprising a recombinant vector according to the invention, in combination with one or more physiologically compatible excipients.

According to a specific embodiment, a method of introducing a nucleic acid according to the invention into a host cell, in particular a host cell obtained from a mammal, in vivo, comprises a step during which a preparation comprising a pharmaceutically compatible vector and a “naked” nucleic acid according to the invention, placed under the control of appropriate regulatory sequences, is introduced by local injection at the level of the chosen tissue, for example a smooth muscle tissue, the “naked” nucleic acid being absorbed by the cells of this tissue.

The invention also relates to the use of a nucleic acid according to the invention, encoding the short or full length ABCA12 protein, for the manufacture of a medicament intended for the prevention and/or treatment in various forms or more particularly for the treatment of subjects affected by a dysfunction in the transport of lipophilic substances or by a pathology located on the chromosome locus 2q34 such as for example the lamellar ichthyosis, the polymorphic congenital cataract, or insulin-dependant diabete mellitus.

The invention also relates to the use of a recombinant vector according to the invention, comprising a nucleic acid encoding any one of the ABCA12 proteins isoforms, for the manufacture of a medicament intended for the prevention and/or treatment of subjects affected by a dysfunction in the transport of lipophilic substances or by a pathology located on the chromosome locus 2q34 such as for example the lamellar ichthyosis, the polymorphic congenital cataract, or insulin-dependant diabete mellitus.

As indicated above, the present invention also relates to the use of a defective recombinant virus according to the invention for the preparation of a pharmaceutical composition for the treatment and/or prevention of pathologies linked to the transport of lipophilic substances and/or linked with deficiencies of the ABCA12 gene.

The invention relates to the use of such a defective recombinant virus for the preparation of a pharmaceutical composition intended for the treatment and/or prevention of a deficiency associated with the transport of lipophilic substances. Thus, the present invention also relates to a pharmaceutical composition comprising one or more defective recombinant viruses according to the invention.

The present invention also relates to the use of cells genetically modified ex vivo with a virus according to the invention, or of producing cells such as viruses, implanted in the body, allowing a prolonged and effective expression in vivo of at least one biologically active ABCA12 proteins.

The present invention shows that it is possible to incorporate a nucleic acid encoding the short or full length ABCA12 polypeptide into a viral vector, and that these vectors make it possible to effectively express a biologically active, mature form. More particularly, the invention shows that the in vivo expression of the ABCA12 gene may be obtained by direct administration of an adenovirus or by implantation of a producing cell or of a cell genetically modified by an adenovirus or by a retrovirus incorporating such a DNA.

Preferably, the pharmaceutical compositions of the invention comprise a pharmaceutically acceptable vehicle or physiologically compatible excipient for an injectable formulation, in particular for an intravenous injection, such as for example into the patient's portal vein. These may relate in particular to isotonic sterile solutions or dry, in particular, freeze-dried, compositions which, upon addition depending on the case of sterilized water or physiological saline, allow the preparation of injectable solutions. Direct injection into the patient's portal vein is preferred because it makes it possible to target the infection at the level of the liver and thus to concentrate the therapeutic effect at the level of this organ.

A “pharmaceutically acceptable vehicle or excipient” includes diluents and fillers which are pharmaceutically acceptable for method of administration, are sterile, and may be aqueous or oleaginous suspensions formulated using suitable dispersing or wetting agents and suspending agents. The particular pharmaceutically acceptable carrier and the ratio of active compound to carrier are determined by the solubility and chemical properties of the composition, the particular mode of administration, and standard pharmaceutical practice.

Any nucleic acid, polypeptide, vector, or host cell of the invention will preferably be introduced in vivo in a pharmaceutically acceptable vehicle or excipient. The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “excipient” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as excipients, particularly for injectable solutions. Suitable pharmaceutical excipients are described in “Remington's Pharmaceutical Sciences” by E. W. Martin.

The pharmaceutical compositions according to the invention may be equally well administered by the oral, rectal, parenteral, intravenous, subcutaneous or intradermal route.

According to another aspect, the subject of the invention is also a preventive and/or curative therapeutic method of treating diseases caused by a deficiency in the transport of lipid substances, comprising administering to a patient or subject a nucleic acid encoding the short or full length ABCA12 polypeptide, said nucleic acid being combined with one or more physiologically compatible vehicles and/or excipients.

In another embodiment, the nucleic acids, recombinant vectors, and compositions according to the invention can be delivered in a vesicle, in particular a liposome (See, Langer, 1990, Science, 249:1527-1533; Treat et al., 1989, Liposomes in the Therapy of Infectious Disease and Cancer, Lopez-Berestein and Fidler (eds.), Liss: New York, pp. 353-365; and Lopez-Berestein, 1989, In: Liposomes in the Therapy of Infectious Disease and Cancer, Lopez-Berestein and Fidler (eds.), Liss: New York, pp. 317-327).

In a further aspect, recombinant cells that have been transformed with a nucleic acid according to the invention and that express high levels of a ABCA12 polypeptide according to the invention can be transplanted in a subject in need of a ABCA12 polypeptide. Preferably autologous cells transformed with ABCA12 encoding nucleic acids according to the invention are transplanted to avoid rejection; alternatively, technology is available to shield non-autologous cells that produce soluble factors within a polymer matrix that prevents immune recognition and rejection.

A subject in whom administration of the nucleic acids, polypeptides, recombinant vectors, recombinant host cells, and compositions according to the invention is performed is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

Preferably, a pharmaceutical composition comprising a nucleic acid, a recombinant vector, or a recombinant host cell, as defined above, will be administered to the patient or subject.

Methods of Screening an Agonist or Antagonist Compound for the ABCA12 Polypeptides

According to another aspect, the invention also relates to various methods of screening compounds or small molecules for therapeutic use which are useful in the treatment of diseases due to a deficiency in the transport of lipid substances or of pathology located on the chromosome locus 2q34 such as for example the lamellar ichthyosis, the polymorphic congenital cataract, or insulin-dependant diabete mellitus.

The invention therefore also relates to the use of any one of ABCA12 polypeptides, or cells expressing the short or full length ABCA12 polypeptide, for screening active ingredients for the prevention and/or treatment of diseases resulting from a dysfunction in ABCA12. The catalytic sites and oligopeptide or immunogenic fragments of ABCA12 polypeptides can serve for screening product libraries by a whole range of existing techniques. The polypeptide fragment used in this type of screening may be free in solution, bound to a solid support, at the cell surface or in the cell. The formation of the binding complexes between of ABCA12 polypeptide fragments and the tested agent can then be measured.

Another product screening technique which may be used in high-flux screenings giving access to products having affinity for the protein of interest is described in application WO84/03564. In this method, applied to ABCA12 proteins, various products are synthesized on a solid surface. These products react with corresponding ABCA12 proteins or fragments thereof and the complex is washed. The products binding the short and/or full length ABCA12 proteins are then detected by methods known to persons skilled in the art. Non-neutralizing antibodies can also be used to capture a peptide and immobilize it on a support.

Another possibility is to perform a product screening method using any one of the ABCA12 neutralizing competition antibodies, the short or full length ABCA12 protein and a product potentially binding the ABCA12 proteins. In this manner, the antibodies may be used to detect the presence of a peptide having a common antigenic unit with ABCA12 polypeptides or proteins.

Of the products to be evaluated for their ability to increase activity of ABCA12, there may be mentioned in particular kinase-specific ATP homologs involved in the activation of the molecules, as well as phosphatases, which may be able to avoid the dephosphorylation resulting from said kinases. There may be mentioned in particular inhibitors of the phosphodiesterase (PDE) theophylline and 3-isobutyl-1-methylxanthine type or the adenylcyclase forskolin activators.

Accordingly, this invention relates to the use of any method of screening products, i.e., compounds, small molecules, and the like, based on the method of translocation of lipophilic substances between the membranes or vesicles, this being in all synthetic or cellular types, that is to say of mammals, insects, bacteria, or yeasts expressing constitutively or having incorporated human ABCA12 encoding nucleic acids. To this effect, labeled lipophilic substances analogs may be used.

Furthermore, knowing that the disruption of numerous transporters have been described (Van den Hazel et al., 1999, J. Biol Chem, 274: 1934-41), it is possible to think of using cellular mutants having a characteristic phenotype and to complement the function thereof with the ABCA12 proteins and to use the whole for screening purposes.

The invention also relates to a method of screening a compound or small molecule active on the transport of lipophilic substances, an agonist or antagonist of the ABCA12 polypeptides, said method comprising the following steps:

a) preparing a membrane vesicle comprising at least the short or full length ABCA12 polypeptide and a lipid substrate comprising a detectable marker;

b) incubating the vesicle obtained in step a) with an agonist or antagonist candidate compound;

c) qualitatively and/or quantitatively measuring release of the lipid substrate comprising a detectable marker; and

d) comparing the release measurement obtained in step b) with a measurement of release of labeled lipophilic substrate by a vesicle that has not been previously incubated with the agonist or antagonist candidate compound.

ABCA12 polypeptides comprise an amino acid sequence selected from SEQ ID NOs: 5 or 6.

According to a first aspect of the above screening method, the membrane vesicle is a synthetic lipid vesicle, which may be prepared according to techniques well known to a person skilled in the art. According to this particular aspect, ABCA12 proteins may be recombinant proteins.

According to a second aspect, the membrane vesicle is a vesicle of a plasma membrane derived from cells expressing at least one of ABCA12 polypeptides. These may be cells naturally expressing the short or full length ABCA12 polypeptide or cells transfected with a nucleic acid encoding at least one ABCA12 polypeptide or with a recombinant vector comprising a nucleic acid encoding at least one ABCA12 polypeptide.

According to a third aspect of the above screening method, the lipid substrate is chosen from cholesterol or phosphatidylcholine.

According to a fourth aspect, the lipid substrate is radioactively labelled, for example with an isotope chosen from ³H or ¹²⁵I.

According to a fifth aspect, the lipid substrate is labelled with a fluorescent compound, such as NBD or pyrene.

According to a sixth aspect, the membrane vesicle comprising the labelled lipophilic substances and one of the ABCA12 polypeptides is immobilized at the surface of a solid support prior to step b).

According to a seventh aspect, the measurement of the fluorescence or radioactivity released by the vesicle is the direct reflection of the activity of lipid substrate transport by the ABCA12 polypeptides.

The invention also relates to a method of screening a compound or small molecule active on the transport of lipid substances, an agonist or antagonist of any one of ABCA12 polypeptides, said method comprising the following steps:

a) obtaining cells, for example a cell line, that, either naturally or after transfecting the cell with any one of ABCA12 encoding nucleic acids, expresses any one of ABCA12 polypeptides;

b) incubating the cells of step a) in the presence of an anion labelled with a detectable marker;

c) washing the cells of step b) in order to remove the excess of the labelled anion which has not penetrated into these cells;

d) incubating the cells obtained in step c) with an agonist or antagonist candidate compound for any one of ABCA12 polypeptides;

e) measuring efflux of the labelled anion; and

f) comparing the value of efflux of the labelled anion determined in step e) with a value of the efflux of a labelled anion measured with cells that have not been previously incubated in the presence of the agonist or antagonist candidate compound of any one of ABCA12 polypeptides.

In a first specific embodiment, any one of the ABCA12 polypeptides comprise an amino acid sequence of SEQ ID NOs: 5 or 6.

According to a second aspect, the cells used in the screening method described above may be cells not naturally expressing, or alternatively expressing at a low level, any one of the ABCA12 polypeptides, said cells being transfected with a recombinant vector according to the invention capable of directing the expression of a nucleic acid encoding any one of the ABCA12 polypeptides.

According to a third aspect, the cells may be cells having a natural deficiency in anion transport, or cells pretreated with one or more anion channel inhibitors such as Verapamil™ or tetraethylammonium.

According to a fourth aspect of said screening method, the anion is a radioactively labelled iodide, such as the salts K¹²⁵I or Na¹²⁵I.

According to a fifth aspect, the measurement of efflux of the labelled anion is determined periodically over time during the experiment, thus making it possible to also establish a kinetic measurement of this efflux.

According to a sixth aspect, the value of efflux of the labelled anion is determined by measuring the quantity of labelled anion present at a given time in the cell culture supernatant.

According to a seventh aspect, the value of efflux of the labelled anion is determined as the proportion of radioactivity found in the cell culture supernatant relative to the total radioactivity corresponding to the sum of the radioactivity found in the cell lysate and the radioactivity found in the cell culture supernatant.

The following examples are intended to further illustrate the present invention but do not limit the invention.

### EXAMPLES

### Example 1

### Search of Human ABCA12 Genes in Sequence Database

Expressed sequence tags (EST) of ABCA1-like genes as described by Allikmets et al. (Hum Mol Genet. October 1996;5(10):1649-55) were used to search Genbank and UniGene nucleotide sequence databases using BLAST2 (Altschul et al, Nucleic Acids Res. Sep. 1, 1997;25(17):3389-402). The main protein sequences databases screened were Swissprot, TrEMBL, Genpept and PIR.

Multiple alignments were generated by GAP software from GCG package and the Dialign2 program (Morgenstern et al, Proc Natl Acad Sci U S A. Oct. 29, 1996;93(22): 12098-103), the FASTA3 package (Pearson et al., Proc Natl Acad Sci U S A. April 1988;85(8):2444-8) and SIM4 (Florea et al, Genome Res., September 1998;8(9):967-74). The specific ABCA motifs used in our process were the TMN, TMC, NBD1 and NBD2 described in the literature (Broccardo et al, Biochim Biophys Acta. Dec. 6, 1999;1461(2):395-404). This corresponds in ABCA1 to residues 630-846 for the N terminal (TMN=exon 14-16) and from 1647-1877 for the C terminal set of membrane spanners (TMC=exon 36-40). The NBD corresponds to the extended nucleotide binding domain, i.e. in ABCA1 it spans from amino acids 885-1152 for the N-terminal one (NBD1=exon 18-22) and 1918-2132 for the C-terminal one (NBD2=exon 42-47).

### Example 2

### 5′ Extension of the Human ABCA12 cDNA

This Example describes the isolation and identification of cDNA molecules encoding the full and short length human ABCA12 proteins. Search in sequence databases evidenced two groups of ESTs that could belong to ABCA12. Linking of these two partial cDNA sequences was performed by RT-PCR. Then 5′ and 3′ extension of the resulting partial ABCA12 cDNA sequence was performed by using a combination of 5′ RACE and RT-PCR on placenta, testis and fetal brain.

Oligonucleotide primers allowing to distinguish the novel ABCA12 gene from other family members, were used to identify specific cDNA transcript by RT-PCR on RNA from various human tissues. The RT-PCR products were either directly sequenced or primarily cloned and then sequenced. In particular, this latter step was carried out for linking of the two partial cDNA sequences in particular. It allowed to evidenced an alternative splicing event corresponding to an additional 230 bp fragment. Then 5′ and 3′ RACE steps were also performed in order to determine the full ORF sequences. The 3′RACE step evidenced two alternative polyadenylation signals. Finally four potential transcripts have thus been identified by RT-PCR and direct sequencing. Mapping experiments revealed a chromosome locus 2q34 localization.

Reverse Transcription

In a total volume of 11.5 μl, 500 ng of mRNA poly(A)+ (Clontech) mixed with 500 ng of oligodT are denaturated at 70° C. for 10 min and then chilled on ice. After addition of 10 units of RNAsin, 10 mM DTT, 0.5 mM dNTP, Superscript first strand buffer and 200 units of Superscript II (Life Technologies), the reaction is incubated for 45 min at 42° C. We used poly(A) mRNA from placenta, testis, and fetal brain.

PCR

Each polymerase chain reaction contained 400 μM each dNTP, 2 units of Thermus aquaticus (Taq) DNA polymerase (Ampli Taq Gold; Perkin Elmer), 0.5 μM each primer, 2.5 mM MgCl₂, PCR buffer and 50 ng of DNA, or about 25 ng of cDNA, or 1/50e of primary PCR mixture. Reactions were carried out for 30 cycles in a Perkin Elmer 9700 thermal cycler in 96-well microtiter plates. After an initial denaturation at 94° C. for 10 min, each cycle consisted of: a denaturation step of 30 s (94° C.), a hybridization step of 30 s (64° C. for 2 cycles, 61° C. for 2 cycles, 58° C. for 2 cycles and 55° C. for 28 cycles), and an elongation step of 1 min/kb (72° C.). PCR ended with a final 72° C. extension of 7 min. In case of RT-PCR, control reactions without reverse transcriptase and reactions containing water instead of cDNA were performed for every sample.

DNA Sequencing

PCR products are analyzed and quantified by agarose gel electrophoresis, purified with a P100 column. Purified PCR products were sequenced using ABI Prism Big Dye terminator cycle sequencing kit (Perkin Elmer Applied Biosystems). The sequence reaction mixture was purified using Microcon-100 microconcentrators (Amicon, Inc., Beverly). Sequencing reactions were resolved on an ABI 377 DNA sequencer (Perkin Elmer Applied Biosystems) according to manufacturer's protocol (Applied Biosystems, Perkin Elmer).

5′ and 3′ Rapid Amplification of cDNA Ends (RACE)

5′ and 3′ RACE analysis were performed using the SMART RACE cDNA amplification kit (Clontech, Palo Alto, Calif.). Human placenta polyA+ RNA (Clontech) was used as template to generate the 5′ and 3′ SMART cDNA libraries according to the manufacturer's instructions. First-amplification primers and nested primers were designed from the cDNA sequence. Amplimers of the nested PCR were cloned. Insert of specific clones are amplified by PCR with universal primers (Rev and −21) and sequenced on both strands. Primers as set forth in SEQ ID NO: 20, 21, 24, 11 and SEQ ID NO: 28, 29 were used to identify 5′ and 3′ ends of ABCA12 respectively.

Primers

Oligonucleotides were selected using Prime from GCG package or Oligo 4 (National Biosciences, Inc.) softwares. Primers were ordered from Life Technologies, Ltd and used without further purification (Table 3).

Physical Mapping

The chromosomal localization of the human ABCA12 gene on the chromosome locus 2q34 was determined by PCR by mapping on the GeneBridge4 radiation hybrid panel (Research Genetics), according to the manufacturer's protocol.

### Example 3

### Electronic Analysis of the Tissue Distribution of the ABCA12 Gene

An electronic analysis of tissue distribution has been performed. The sequence of the transcript (SEQ ID N° 1-4) matches with 6 different Incyte templates numbered 54714.1, 1337198.1, 88352.1, 1337102.1, 222677.1, and 385780.1 (Incyte template September 2000 database [LGTemplatesSEP2000]) that are constituted of 5, 1, 2, 1, 14, and 1 ESTs respectively. The tissue origin of all these ESTs may suggest a preferential skin/epithelial cell expression (12 ESTs over 24 come from squamous cells, epithelial cells, or skin) of ABCA12 transcript.

### Example 4

### Construction of the Expression Vector Containing the ABCA12 Nucleic Acids in Mammalian Cells

The ABCA12 gene may be expressed in mammalian cells. A typical eukaryotic expression vector contains a promoter which allows the initiation of the transcription of the mRNA, a sequence encoding the protein, and the signals required for the termination of the transcription and for the polyadenylation of the transcript. It also contains additional signals such as enhancers, the Kozak sequence and sequences necessary for the splicing of the mRNA. An effective transcription is obtained with the early and late elements of the SV40 virus promoters, the retroviral LTRs or the CMV virus early promoter. However, cellular elements such as the actin promoter may also be used. Many expression vectors may be used to carry out the present invention, an example of such a vector is pcDNA3 (Invitrogen).

### Example 5

### Production of Normal and Mutated ABCA 12 Polypeptides

The normal ABCA12 polypeptides encoded by complete corresponding cDNAs whose isolation is described in Example 2, or mutated ABCA12 polypeptides whose complete cDNA may also be obtained according to the techniques described in Example 2, may be easily produced in a bacterial or insect cell expression system using the baculovirus vectors or in mammalian cells with or without the vaccinia virus vectors. All the methods are now widely described and are known to persons skilled in the art. A detailed description thereof will be found for example in F. Ausubel et al. (1989, Current Protocols in Molecular Biology, Green Publishing Associates and Wiley Interscience, N.Y).

### Example 6

### Production of an Antibody Directed Against a Mutated ABCA12 Polypeptide

The antibodies in the present invention may be prepared by various methods (Current Protocols In Molecular Biology Volume 1 edited by Ausubel et al., Massachusetts General Hospital Harvard Medical School, chapter 11, 1989). For example, the cells expressing a polypeptide of the present invention are injected into an animal in order to induce the production of serum containing the antibodies. In one of the methods described, the proteins are prepared and purified so as to avoid contaminations. Such a preparation is then introduced into the animal with the aim of producing polyclonal antisera having a higher activity.

In the preferred method, the antibodies of the present invention are monoclonal antibodies. Such monoclonal antibodies may be prepared using the hybridoma technique (Köhler et al, 1975, Nature, 256:495; Köhler et al, 1976, Eur. J. Immunol. 6:292; Köhler et al, 1976, Eur. J. Immunol., 6:511; Hammeling et al., 1981, Monoclonal Antibodies and T-Cell Hybridomas, Elsevier, N.Y., pp. 563-681). In general, such methods involve immunizing the animal (preferably a mouse) with a polypeptide or better still with a cell expressing the polypeptide. These cells may be cultured in a suitable tissue culture medium. However, it is preferable to culture the cells in an Eagle medium (modified Earle) supplemented with 10% fetal bovine serum (inactivated at 56° C.) and supplemented with about 10 g /l of nonessential amino acids, 1000 U/ml of penicillin and about 100 μg/ml of streptomycin.

The splenocytes of these mice are extracted and fused with a suitable myeloma cell line. However, it is preferable to use the parental myeloma cell line (SP20) available from the ATCC. After fusion, the resulting hybridoma cells are selectively maintained in HAT medium and then cloned by limiting dilution as described by Wands et al. (1981, Gastroenterology, 80:225-232). The hybridoma cells obtained after such a selection are tested in order to identify the clones secreting antibodies capable of binding to the polypeptide.

Moreover, other antibodies capable of binding to the polypeptide may be produced according to a 2-stage procedure using anti-idiotype antibodies such a method is based on the fact that the antibodies are themselves antigens and consequently it is possible to obtain an antibody recognizing another antibody. According to this method, the antibodies specific for the protein are used to immunize an animal, preferably a mouse. The splenocytes of this animal are then used to produce hybridoma cells, and the latter are screened in order to identify the clones which produce an antibody whose capacity to bind to the specific antibody-protein complex may be blocked by the polypeptide. These antibodies may be used to immunize an animal in order to induce the formation of antibodies specific for the protein in a large quantity.

It is preferable to use Fab and F(ab′)2 and the other fragments of the antibodies of the present invention according to the methods described here. Such fragments are typically produced by proteolytic cleavage with the aid of enzymes such as Papaïn (in order to produce the Fab fragments) or Pepsin (in order to produce the F(ab′)2 fragments). Otherwise, the secreted fragments recognizing the protein may be produced by applying the recombinant DNA or synthetic chemistry technology.

For the in vivo use of antibodies in humans, it would be preferable to use “humanized” chimeric monoclonal antibodies. Such antibodies may be produced using genetic constructs derived from hybridoma cells producing the monoclonal antibodies described above. The methods for producing the chimeric antibodies are known to persons skilled in the art (for a review, see: Morrison (1985, Science 229:1202); Oi et al., (1986, Biotechnique, 4:214); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al; (1984, Nature, 312:643); and Neuberger et al., (1985, Nature, 314:268).

### Example 7

### Determination of Polymorphisms/Mutations in the ABCA12 Gene

The detection of polymorphisms or mutations in the sequences of the transcripts or in the genomic sequence of the ABCA12 gene may be carried out according to various protocols. The preferred method is direct sequencing.

For patients from whom it is possible to obtain an mRNA preparation, the preferred method consists in preparing the cDNAs and sequencing them directly. For patients for whom only DNA is available, and in the case of a transcript where the structure of the corresponding gene is unknown or partially known, it is necessary to precisely determine its intron-exon structure as well as the genomic sequence of the corresponding gene. This therefore involves, in a first instance, isolating the genomic DNA BAC or cosmid clone(s) corresponding to the transcript studied, sequencing the insert of the corresponding clone(s) and detemrining the intron-exon structure by comparing the cDNA sequence to that of the genomic DNA obtained.

The technique of detection of mutations by direct sequencing consists in comparing the genomic sequences of the ABCA12 gene obtained from homozygotes for the disease or from at least 8 individuals (4 individuals affected by the pathology studied and 4 individuals not affected) or from at least 32 unrelated individuals from the studied population. The sequence divergences constitute polymorphisms. All those modifying the amino acid sequence of the wild-type protein isoforms may be mutations capable of affecting the function of said protein which it is preferred to consider more particularly for the study of cosegregation of the mutation and of the disease (denoted genotype-phenotype correlation) in the pedigree, or of a pharmacological response to a therapeutic molecule in the pharmacogenomic studies, or in the studies of case/control association for the analysis of the sporadic cases.

### Example 8

### Identification of a Causal Gene for a Disease Linked to Causal Mutation or a Transcriptional Difference of the ABCA12 Gene

Among the mutations identified according to the method described in Example 7, all those associated with the disease phenotype are capable of being causal. Validation of these results is made by sequencing the gene in all the affected individuals and their relations (whose DNA is available).

Moreover, Northern blot or RT-PCR analysis, according to the methods described in Example 2, using RNA specific to affected or nonaffected individuals makes it possible to detect notable variations in the level of expression of the gene studied, in particular in the absence of transcription of the gene.

### Example 9

### Construction of Recombinant Vectors Comprising ABCA12 Nucleic Acids

Synthesis of a Nucleic Acid Encoding a Human ABCA12 Protein:

Total RNA (500 ng) isolated from a human cell (for example, placental tissue, Clontech, Palo Alto, Calif., USA, or THP1 cells) may be used as source for the synthesis of the cDNA of the human ABCA12 gene. Methods to reverse transcribe mRNA to cDNA are well known in the art. For example, one may use the system “Superscript one step RT-PCR (Life Technologies, Gaithersburg, Md., USA).

Oligonucleotide primers specific for ABCA12 cDNAs may be used for this purpose, containing sequences as set forth in any of SEQ ID NO: 7-38. These oligonucleotide primers may be synthesized by the phosphoramidite method on a DNA synthesizer of the ABI 394 type (Applied Biosystems, Foster City, Calif., USA).

Sites recognized by the restriction enzyme NotI may be incorporated into the amplified ABCA12 cDNAs to flank the cDNA region desired for insertion into the recombinant vector by a second amplification step using 50 ng of human ABCA12 cDNAs as template, and 0.25 μM of the ABCA12 specific oligonucleotide primers used above containing, at their 5′ end, the site recognized by the restriction enzyme NotI (5′-GCGGCCGC-3′), in the presence of 200 μM of each of said dideoxynucleotides dATP, dCTP, dTTP and dGTP as well as the Pyrococcus furiosus DNA polymerase (Stratagene, Inc. La Jolla, Calif., USA).

The PCR reaction may be carried out over 30 cycles each comprising a step of denaturation at 95° C. for one minute, a step of renaturation at 50° C. for one minute and a step of extension at 72° C. for two minutes, in a thermocycler apparatus for PCR (Cetus Perkin Elmer Norwalk, Conn., USA).

Cloning of the cDNA of the Human ABCA12 Gene into an Expression Vector:

The human ABCA12 cDNA inserts may then be cloned into the NotI restriction site of an expression vector, for example, the pCMV vector containing a cytomegalovirus (CMV) early promoter and an enhancer sequence as well as the SV40 polyadenylation signal (Beg et al., 1990, PNAS, 87:3473; Applebaum-Boden, 1996, JCI 97), in order to produce an expression vector designated pABCA12.

The sequence of the cloned cDNA can be confirmed by sequencing on the two strands using the reaction set “ABI Prism Big Dye Terminator Cycle Sequencing ready” (marketed by Applied Biosystems, Foster City, Calif., USA) in a capillary sequencer of the ABI 310 type (Applied Biosystems, Foster City, Calif., USA).

Construction of a Recombinant Adenoviral Vector Containing the cDNA of the Human ABCA12 Gene:

Modification of the Expression Vector pCMV-β:

The β-galactosidase cDNA of the expression vector pCMV-β (Clontech, Palo Alto, Calif., USA, Gene Bank Accession No. UO2451) may be deleted by digestion with the restriction endonuclease NotI and replaced with a multiple cloning site containing, from the 5′ end to the 3′ end, the following sites: NotI, AscI, RsrII, AvrII, SwaI, and NotI, cloned at the region of the NotI restriction site. The sequence of this multiple cloning site is: 5′-CGGCCGCGGCGCGCCCGGACCGCCTAGGATTTAAATCGCGGCCCGCG-3′.

The DNA fragment between the EcoRI and SanI sites of the modified expression vector pCMV may be isolated and cloned into the modified XbaI site of the shuttle vector pXCXII (McKinnon et al., 1982, Gene, 19:33; McGrory et al., 1988, Virology, 163:614).

Modification of the Shuttle Vector pXCXII:

A multiple cloning site comprising, from the 5′ end to the 3 end the XbaI, EcoRI, SfiI, PmeI, NheI, SrfI, PacI, SalI and XbaI restriction sites having the sequence: 5′CTCTAGAATTCGGCCTCCGTGGCCGTTTAAACGCTAGCGCCCGGGCTTAATTAAGTCGACTCTAGAGC-3′, may be inserted at the level of the XbaI site (nucleotide at position 3329) of the vector pXCXII (McKinnon et al., 1982, Gene 19:33; McGrory et al., 1988, Virology, 163:614).

The EcoRI-SalI DNA fragment isolated from the modified vector pCMV-β containing the CMV promoter/enhancer, the donor and acceptor splicing sites of FV40 and the polyadenylation signal of SV40 may then be cloned into the EcoRI-SalI site of the modified shuttle vector pXCX, designated pCMV-11.

Preparation of the Shuttle Vector pAD12-ABCA:

The human ABCA12 cDNAs are obtained by an RT-PCR reaction, as described above, and cloned at the level of the NotI site into the vector pCMV-12, resulting in the obtaining of the vector pCMV-ABCA12.

Construction of the ABC12 Recombinant Adenovirus:

The recombinant adenovirus containing the human ABCA12 cDNAs may be constructed according to the technique described by McGrory et al. (1988, Virology, 163:614).

Briefly, the vector pAD12-ABCA is cotransfected with the vector tGM17 according to the technique of Chen and Okayama (1987, Mol Cell Biol., 7:2745-2752).

Likewise, the vector pAD12-Luciferase was constructed and cotransfected with the vector pJM17.

The recombinant adenoviruses are identified by PCR amplification and subjected to two purification cycles before a large-scale amplification in the human embryonic kidney cell line HEK 293 (American Type Culture Collection, Rockville, Md., USA).

The infected cells are collected 48 to 72 hours after their infection with the adenoviral vectors and subjected to five freeze-thaw lysing cycles.

The crude lysates are extracted with the aid of Freon (Halocarbone 113, Matheson Product, Scaucus, N.J. USA), sedimented twice in cesium chloride supplemented with 0.2% murine albumine (Sigma Chemical Co., St Louis, Mo., USA) and dialysed extensively against buffer composed of 150 nM NaCl, 10 mM Hepes (pH 7,4), 5 mM KCl, 1 mM MgCl₂, and 1 mM CaCl₂.

The recombinant adenoviruses are stored at −70° C. and titrated before their administration to animals or their incubation with cells in culture.

The absence of wild-type contaminating adenovirus is confirmed by screening with the aid of PCR amplification using oligonucleotide primers located in the structural portion of the deleted region.

Validation of the Expression of the Human ABCA12 cDNAs:

Polyclonal antibodies specific for a human ABCA12 polypeptide may be prepared as described above in rabbits and chicks by injecting a synthetic polypeptide fragment derived from an ABC12 protein, comprising all or part of an amino acid sequence as described in SEQ ID NO: 5 or 6. These polyclonal antibodies are used to detect and/or quantify the expression of the human ABCA12 gene in cells and animal models by immunoblotting and/or immunodetection.

Expression in vitro of the Human ABCA12 cDNAs in Cells:

Cells of the HEK293 line and of the COS-7 line (American Tissue Culture Collection, Bethesda, Md., USA), as well as fibroblasts in primary culture are transfected with the expression vector pCMV-ABCA12 (5-25 μg) using Lipofectamine (BRL, Gaithersburg, Md., USA) or by coprecipitation with the aid of calcium chloride (Chen et al., 1987, Mol Cell Biol., 7:2745-2752).

These cells may also be infected with the vector pABCA12-AdV (Index of infection, MOI=10).

The expression of the human ABCA12 gene may be monitored by immunoblotting using transfected and/or infected cells.

Expression in vivo of the Human ABCA12 Gene in Various Animal Models:

An appropriate volume (100 to 300 μl) of a medium containing the purified recombinant adenovirus (pABCA-AdV or pLucif-AdV) containing from 10⁸ to 10⁹ lysis plaque-forming units (pfu) are infused into the Saphenous vein of mice (C57BL/6, both control mice and models of transgenic or knock-out mice) on day 0 of the experiment.

The evaluation of the physiological role of the ABCA12 protein in the transport of lipid substances is carried out by determining the total quantity of lipid substances before (day zero) and after (days 2, 4, 7, 10, 14) the administration of the adenovirus.

Kinetic studies with the aid of radioactively labelled products are carried out on day after the administration of the vectors rLucif-AdV and rABCA-AdV in order to evaluate the effect of the expression of ABCA12 on the transport of lipid substances.

Furthermore, transgenic mice and rabbits overexpressing the ABCA12 gene may be produced, in accordance with the teaching of Vaisman (J Biol Chem., May 19, 1995;270(20):12269-75) and Hoeg (J Biol Chem., Feb. 23, 1996;271(8):4396-402) using constructs containing the human ABCA12 cDNAs under the control of endogenous promoters such as CMV or apoE.

The evaluation of the long-term effect of the expression of ABCA12 on the kinetics of the lipids may be carried out as described above.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. An isolated nucleic acid comprising any one of SEQ ID NOs: 1-4, or a complementary nucleotide sequence thereof.

2. An isolated nucleic acid comprising at least eight consecutive nucleotides of a nucleotide sequence of any one of SEQ ID NOs: 1-4, or a complementary nucleotide sequence thereof.

3. An isolated nucleic acid comprising at least 80% nucleotide identity with a nucleic acid comprising any one of SEQ ID NOs: 1-4, or a complementary nucleotide sequence thereof.

4. The isolated nucleic acid according to claim 3, wherein the nucleic acid comprises an 85%, 90%, 95%, or 98% nucleotide identity with the nucleic acid comprising any one of SEQ ID NOs: 1-4, or a complementary nucleotide sequence thereof.

5. An isolated nucleic acid that hybridizes under high stringency conditions with a nucleic acid comprising any one of SEQ ID NOs: 1-4, or a complementary nucleotide sequence thereof.

6. An isolated nucleic acid comprising a nucleotide sequence as depicted in any one of SEQ ID NOs: 1-4, or of a complementary nucleotide sequence thereof.

7. A nucleotide probe or primer specific for the ABCA12 gene, wherein the nucleotide probe or primer comprises at least 15 consecutive nucleotides of a nucleotide sequence of any one of SEQ ID NOs: 1-4, or of a complementary nucleotide sequence thereof.

8. A nucleotide probe or primer specific for the ABCA12 gene, wherein the nucleotide probe or primer comprises a nucleotide sequence of any one of SEQ ID NO: 7-38, or a complementary nucleotide sequence thereof.

9. The nucleotide probe or primer according to any of claim 7 or 8, wherein the nucleotide probe or primer comprises a marker compound.

10. A method of amplifying a region of the nucleic acid according to claim 1, wherein the method comprises:
a) contacting the nucleic acid with two nucleotide primers, wherein the first nucleotide primer hybridizes at a position 5′ of the region of the nucleic acid, and the second nucleotide primer hybridizes at a position 3′ of the region of the nucleic acid, in the presence of reagents necessary for an amplification reaction; and
b) detecting the amplified nucleic acid region.

11. A method of amplifying a region of the nucleic acid according to claim 10, wherein the two nucleotide primers are selected from the group consisting of
a) a nucleotide primer comprising at least 15 consecutive nucleotides of a nucleotide sequence of any one of SEQ ID NOs: 1-4, or of a complementary nucleotide sequence,
b) a nucleotide primer comprising a nucleotide sequence of any one of SEQ ID 10 NOs: 7-38, or a complementary sequence thereof.

12. A kit for amplifying the nucleic acid according to claim 1, wherein the kit comprises:
a) two nucleotide primers whose hybridization position is located respectively 5′ and 3′ of the region of the nucleic acid; and optionally,
b) reagents necessary for an amplification reaction.

13. The kit according to claim 12, wherein the two nucleotide primers are selected from the group consisting of
a) a nucleotide primer comprising at least 15 consecutive nucleotides of a nucleotide sequence of any one of SEQ ID NOs: 1-4, or of a complementary nucleotide sequence,
b) a nucleotide primer comprising a nucleotide sequence of any one of SEQ ID NOs: 7-38, or a complementary sequence thereof.

14. A method of detecting a nucleic acid according to claim 1, wherein the method comprises:
a) contacting the nucleic acid with a nucleotide probe selected from the group consisting of
1) a nucleotide probe comprising at least 15 consecutive nucleotides of a nucleotide sequence of any one of SEQ ID NOs: 1-4, or a complementary nucleotide sequence thereof,
2) a nucleotide probe as in any one of claims 7-9,
3) a nucleotide probe comprising a nucleotide sequence of any one of SEQ ID NOs: 7-38, or a complementary nucleotide sequence thereof, and
b) detecting a complex formed between the nucleic acid and the probe.

15. The method of detection according to claim 14, wherein the probe is immobilized on a support.

16. A kit for detecting the nucleic acid according to claim 1, wherein the kit comprises
a) a nucleotide probe selected from the group consisting of 1) a nucleotide probe comprising at least 15 consecutive nucleotides of a nucleotide sequence of any one of SEQ ID NOs: 1-4, or a complementary nucleotide sequence thereof, 2) a nucleotide primer as in any one of claim 7 or 9, 3) a nucleotide probe comprising a nucleotide sequence of any one of SEQ ID NOs: 7-38, or a complementary nucleotide sequence thereof, and optionally,
b) reagents necessary for a hybridization reaction.

17. The kit according to claim 16, wherein the probe is immobilized on a support.

18. A recombinant vector comprising the nucleic acid according claim 1.

19. The vector according to claim 18, wherein the vector is an adenovirus.

20. A recombinant host cell comprising the recombinant vector according to claim 19.

21. A recombinant host cell comprising the nucleic acid according claim 1.

22. An isolated nucleic acid encoding a polypeptide comprising an amino acid sequence of any one of SEQ ID NO: 5 or 6.

23. A recombinant vector comprising the nucleic acid according to claim 22.

24. A recombinant host cell comprising the nucleic acid according to claim 22.

25. A recombinant host cell comprising the recombinant vector according to claim 23.

26. An isolated polypeptide selected from the group consisting of
a) a polypeptide comprising an amino acid sequence of any one of SEQ ID NOs: 5 or 6,
b) a polypeptide fragment or variant of a polypeptide comprising an amino acid sequence of any one of SEQ ID NOs: 5 or 6, and
c) a polypeptide homologous to a polypeptide comprising amino acid sequence of any one of SEQ ID NO: 5 or 6.

27. An antibody directed against the isolated polypeptide according to claim 26.

28. The antibody according to claim 27, wherein the antibody comprises a detectable compound.

29. A method of detecting a polypeptide, wherein the method comprises
a) contacting the polypeptide with an antibody according to claim 28; and
b) detecting an antigen/antibody complex formed between the polypeptide and the antibody.

30. A diagnostic kit for detecting a polypeptide, wherein the kit comprises
a) the antibody according to claim 28; and
b) a reagent allowing detection of an antigen/antibody complex formed between the polypeptide and the antibody.

31. A pharmaceutical composition comprising the nucleic acid according to claim 1 and a physiologically compatible excipient.

32. A pharmaceutical composition comprising the recombinant vector according to claim 23 and a physiologically compatible excipient.

33. Use of a recombinant vector according to claim 18 for the manufacture of a medicament for the prevention and/or treatment of a subject affected by a dysfunction in the lipophilic subtance transport.

34. Use of an isolated ABCA12 polypeptide comprising an amino acid sequence of SEQ ID NO: 5 or 6 for the manufacture of a medicament intended for the prevention and/or treatment of a subject affected by a dysfunction in the lipophilic subtance transport or by a pathology located on the chromosome locus 2q34 such as for example the lamellar ichthyosis, the polymorphic congenital cataract, or insulin-dependant diabete mellitus.

35. A pharmaceutical composition comprising a polypeptide comprising an amino acid sequence of any one of SEQ ID NOs: 5 or 6, and a physiologically compatible excipient.

36. Use of an ABCA12 polypeptide comprising an amino acid sequence of any one of SEQ ID NOs: 5 or 6 for screening an active ingredient for the prevention or treatment of a disease resulting from a dysfunction in the lipophilic subtance transport or of a pathology located on the chromosome locus 2q34 such as for example the lamellar ichthyosis, the polymorphic congenital cataract, or insulin-dependant diabete mellitus.

37. Use of a recombinant host cell expressing an ABCA12 polypeptide comprising an amino acid sequence of any one of SEQ ID NOs: 5 or 6, for screening an active ingredient for the prevention or treatment of a disease resulting from a dysfunction in the lipophilic subtance transport.

38. A method of screening a compound active on the transport of lipid substance, an agonist, or an antagonist of ABCA12 polypeptides, wherein the method comprises
a) preparing a membrane vesicle comprising ABCA12 polypeptide having SEQ ID NOs: 4 or 5 and a lipid substrate comprising a detectable marker;
b) incubating the vesicle obtained in step a) with an agonist or antagonist candidate compound;
c) qualitatively and/or quantitatively measuring a release of the lipid substrate comprising the detectable marker; and
d) comparing the release of the lipid substrate measured in step b) with a measurement of a release of a labeled lipid substrate by a membrane vesicle that has not been previously incubated with the agonist or antagonist candidate compound.

39. A method of screening an agonist or an antagonist of ABCA12 polypeptides, wherein the method comprises
a) incubating a cell that expresses at least a ABCA12 polypeptide having SEQ ID NOs: 4 or 5 with an anion labeled with a detectable marker;
b) washing the cell of step a) whereby excess labeled anion that has not penetrated into the cell is removed;
c) incubating the cell obtained in step b) with an agonist or antagonist candidate compound for the ABCA12 polypeptide;
d) measuring efflux of the labeled anion from the cell; and
e) comparing the efflux of the labeled anion determined in step d) with efflux of a labeled anion measured with a cell that has not been previously incubated with the agonist or antagonist candidate compound.

40. An implant comprising the recombinant host cell according to claim 24.


SEQUENCE LISTING (38 sequences)

SEQ ID NO: 1 - DNA, 9112 bp, Homo sapiens
  n (1)..(9112): n = a, t, g, or c
gaagagttgattgagaagtgcctcttggttaaggattaaccacagggaaaaatccagcagaaacagaagaactgtgggtttcttaccccagccctcaaggaagctatgccgtgaaaggggtactgatacactgacatacagcaagttggacggggcatcagttcttcatttgtggagtggagaaaagaagaggaaatctctcatttggggcatttgaaggatggcttccctgtttcatcagcttcagatcctggtctggaaaaattggctaggtgtaaaaaggcagccgctttggacacttgtcttgatcttatggccagtcattattttcataattttggctattactcggaccaaatttcctccaactgcaaaaccaacttgttacctcgcacctcgaaaccttcctagtactggattctttccattcctgcagaccctactctgtgacacagactctaaatgcaaagacacaccctatggcccacaagatctgcttcgtaggaaaggaattgatgatgcactatttaaagacagtgagattctgagaaagtcatccaacctggataaggacagcagtttatcattccagagcacccaagttccagaaagaaggcatgcatcactagccacagtatttcccagtccaagttctgatttggaaatccccggaacatatactttcaatggcagtcaagtgctcgcacgaattcttggcttggaaaagctgttaaagcaaaattcaacttcagaagatatacgaagagaactatgtgacagctattcaggatacattgtggatgatgccttctcttggacctttctaggaagaaatgtttttaacaaattttgcctttctaacatgacccttttagagtcttctctccaagaactaaacaaacagttctcccagctatccagtgaccccaacaatcagaagatagtgtttcaggaaatagtcagaatgctgtctttcttctcacaagtgcaagagcagaaagctgtgtggcagcttctgtctagttttccaaatgtgtttcagaatgacacatcactaagcaatctatttgatgttcttcgaaaggcaaacagtgtgctgctggttgtgcagaaggtttatccacgttttgcaactaacgaaggtttcagaaccctccagaagtctgttaaacatctgctgtacactctggactccccagctcaaggtgactccgataatataacgcatgtgtggaatgaggatgatggacagaccttatctccaagcagtctggctgcacagctcctaattctggaaaactttgaagatgccctcttaaatatatcagcaaatagtccttatattccttacttggcatgtgtgagaaatgtgactgacagtttggccagaggttcaccagaaaatctaagactcctgcagtccacaatacgatttaaaaaatcttttcttcgcaatggttcctatgaagattactttcctccagttcctgaagtcctaaaatcaaaactgtctcaacttcgaaacttgaccgaacttctttgtgaatctgaaactttcagtttgatagagaagtcatgccagctctctgatatgagctttgggagcctgtgtgaagaaagtgagtttgatctgcaactcctcgaagcggcagagctgggcaccgaaatagcagccagcttactgtaccatgacaatgtcatatctaaaaaagtgagagatttgctgactggagatccaagcaaaattaatttaaatatggatcagtttctagaacaggcactgcaaatgaattacttggaaaatatcactcagttaataccgatcatagaagccatgctgcatgtcaataacagtgcagatgcttctgaaaagccaggtcagttactagaaatgtttaaaaatgttgaagagctgaaagaagatttaaggagaacaacaggaatgtccaacaggactattgacaagttgctggccattcccatccctgataatagagctgagattatttctcaggtgttctggctgcattcctgtgatactaatatcaccactcccaaactagaagatgcaatgaaagaattctgcaacctgtctctttcagagagatcccggcagtcttacctcatcggactcacccttctgcactacttaaacatttacaacttcacagacaaggtgtttttcccgaggaaagatcaaaagccagtagaaaagatgatggagctcttcataagactaaaagagattctcaatcagatggcttctggcacacatccgctgctagacaaaatgagatccctgaagcaaatgcatctgcccagaagtgttccattaacacaggcaatgtacagaagcaaccgaatgaacacaccacaaggatcatttagcaccatctcccaagcattatgttctcaaggaattaccactgaatatttaactgccatgctgccctcttcccagaggccaaaaggcaaccacaccaaggattttttgacttataaattaactaaagagcaaattgcttcaaaatatggaattcccataaataccacaccattttgcttctccctttataaagacatcattaacatgcccgctggacctgtgatttgggctttcttgaaacctatgttgttgggaagaattttgcatgcaccatataacccagtcacaaaggcaataatggaaaagtccaatgtaactctgagacagctggcggaattaagagaaaaatctcaagagtggatggataagtcgccacttttcatgaattccttccatctgttaaaccaggcaattccaatgctccagaatactctaaggaacccttttgtgcaagtttttgtaaagttctccgtgggactcgatgctgttgaactattgaaacagatagatgaactcgatattctaagactgaaattagagaacaacattgacatcatcgatcagcttaacacactatcttccctgacagtaaatatttcctcttgtgtattatatgaccgtattcaggcagcaaaaaccatagatgaaatggagagagaggctaaaaggctctacaaaagcaacgaactctttggaagtgttatttttaagcttccttctaacagaagctggcacagaggctatgactctggaaatgtctttcttcctcctgtcataaaatataccatccggatgagtctcaagaccgcacagaccacaagaagcctaagaaccaagatttgggctccagggccacacaattctccatcacacaaccagatctatggcagggcttttatttatttacaggatagtattgaaagagcaatcattgaattgcaaactggaaggaactcccaggaaatagcagtccaggttcaagcaattccttatccctgcttcatgaaagacaacttcctaaccagtgtctcttattctcttccaattgtgcttatggttgcctgggttgtatttatagctgcctttgtaaaaaagcttgtctatgagaaagacctccggcttcatgagtacatgaagatgatgggtgtgaactcctgcagccatttctttgcctggcttatagagagtgttggatttttactggttaccatcgtgatcctcatcattatactcaagtttggcaatattcttcctaaaacaaatgggttcattttgttcctgtatttttcggactacagcttctcggttattgccatgagctatcttatcagtgtcttcttcaacaacaccaacattgcagctctgatcggaagcctcatctacatcattgccttctttccatttattgttctggttacagtggagaatgagttgagctatgtattgaaagtgttcatgagcctgctgtccccaacagcattcagctatgcaagccaatacattgcacgatacgaagaacagggcattggtcttcagtgggaaaatatgtacacctccccggttcaggatgacaccacctcatttggctggctgtgctgtctaatcctagctgactctttcatttatttccttattgcttggtatgtcaggaatgtcttcccagggacatacggtatggcagctccctggtattttccaattcttccttcctattggaaggagcgatttgggtgtgcagaggtgaagcctgagaagagcaatggcctcatgtttactaacatcatgatgcagaacaccaacccatctgccagtcctgaatacatgttttcctctaacatcgagcctgaacctaaagatctcacagtcggggttgccctgcatggggtcacaaagatctatggctcaaaagttgctgttgataacctcaatctgaacttttatgaagggcatattacttcattgctggggcccaatggagctgggaaaactactaccatttccatgttaactgggctgtttggggcctcagcaggcaccatttttgtatatggaaaagatatcaaaacagacctacacacggtacggaagaacatgggagtctgtatgcagcacgacgtcttgttcagttacctcactactaaggagcaccttctcctatatggttccatcaaagttcctcactggactaaaaagcagctccacgaggaagtaaaaaggactttaaaagatactggactatatagccatcgtcataagagagttggaacactgtcaggaggcatgaagaggaagttatctatatccatagctctcattggtggatcaagggtagtaattttggatgaaccatctactggagttgacccatgttctcgccgaagtatatgggatgttatatccaagaacaaaactgccagaacaatcattctgtcaacgcaccacttggacgaggctgaagtgctgagtgaccgcatcgccttcctggagcagggtgggcttaggtgctgtgggtccccattttacctcaaggaagcctttggcgatgggtatcacctcacgcttaccaagaagaagagtccaaatttaaatgcaaatgcagtatgtgacaccatggccgtgacagcaatgatccaatcacatctccccgaagcctacctcaaggaggatattgggggagagcttgtttatgtacttcctccattcagcaccaaagtctcaggggcctacctgtcactcctacgggcactcgacaatggcatgggtgacctcaacatcgggtgctacggcatttcagataccaccgtggaggaggtctttctgaacttgaccaaagagtcacaaaaaaatagtgctatgagtcttgagcacttaacacaaaagaaaattgggaattccaatgccaatggcatctcaactcctgacgatttatctgtgagcagcagcaatttcacagacagagatgacaaaatcctgacaagaggagagaggctggatggctttggactgttgctgaagaagatcatggctatactcatcaagaggttccaccacacccgcaggaactggaaaggtctcattgctcaggttatcctccccatcgtctttgttaccactgccatgggccttggcacactgagaaattccagcaacagttatccagagattcagatctccccctctctttatggtacctccgaacagacagccttctatgctaattatcacccgagcacggaagcacttgtctcagcaatgtgggacttccctggaattgacaacatgtgtctgaacaccagtgatctacagtgtttaaacaaagacagtctggaaaaatggaacaccagtggagaacccatcactaattttggtgtttgctcctgctcagaaaatgtccaggaatgtcctaaatttaactattccccaccgcacagaagaacttactcatcccaggtaatttataacctcactgggcaacgagtggaaaattatcttatatcaactgcaaatgagtttgtccaaaaaagatatggaggttggagttttgggctgcctttgacaaaagaccttcgttttgatataacaggagtccctgccaatagaacacttgccaaggtatggtatgatccagaaggctatcactcccttccagcttacctcaacagcctgaataatttccttctgcgagttaacatgtcaaaatacgatgctgcccgacatggcatcatcatgtatagccatccttatccaggagtgcaagaccaagaacaagccacaatcagcagtttaatcgatattttagtggcactgtctatcttgatgggctactctgtcaccaccgccagctttgtcacctatgttgtaagggaacatcaaaccaaagccaaacagttgcagcacatttcaggcattggcgtgacatgctactgggtaacaaacttcatttatgacatggttttctacttggtgcctgtagcgttttcaattggtatcattgcgattttcaaattacctgcattctacagtgaaaacaacctaggcgctgtatctctcctacttctcctgtttgggcatgcaacattttcctggatgtacttgctggctgggctcttccatgaaacaggaatggccttcatcacttacgtctgtgtcaacttgttttttggcattaattccattgtttccctgtcagtggtatactttctttccaaggaaaagcctaatgatccgactttagaacttatttctgaaaccctcaagcgcattttcctgattttcccacaattctgttttggctacggtttgattgaactttctcaacaacagtcggtcctagacttcttaaaagcatatggagtggaatacccaaatgaaacctttgagatgaataaactaggtgcaatgtttgtggctttggtttctcagggcaccatgtttttttccttgcgactcttaatcaacgaatccctgataaagaaactcaggcttttcttcagaaaatttaattcttcacatgtaagggagacaatagatgaggatgaagatgtgcgggctgagagattaagagttgagagtggtgcagctgaatttgacttggtccaactttattgtctcacaaagacctaccaacttatccacaaaaagattatagctgtaaacaacatcagcatcgggatacctgctggagagtgttttgggcttcttggagtgaatggagcaggaaagaccactatattcaagatgctgacaggagacatcattccttcaagtggaaacattctgatcagaaataagaccggatctctgggtcacgttgattctcacagctcattagttggctactgtcctcaggaagatgccttagatgacctggtaactgtggaagaacatttgtatttctatgccagggtacatggaattccagaaaaggatattaaagaaactgttcataaactccttaggagacttcacctgatgcccttcaaggacagagctacctctatgtgcagttatggcacaaaaagaaaattatccactgcactggccttgatagggaaaccttccattctactgctggatgagccgagctctggcatggatccgaagtcgaaacggcacctctggaagatcatttcagaagaagtacagaacaaatgttccgtcatcctcacatctcacagcatggaagaatgtgaagctctctgtaccaggttggccattatggtgaatggaaagtttcaatgtattggatctttgcagcacataaagagcaggtttggacgaggatttactgtcaaagttcacttgaagaataacaaagtgaccatggagaccctcacaaagttcatgcagctgcactttccaaaaacatacttaaaagatcagcacctcagcatgctagagtatcatgtaccagtcacagcaggaggagtcgcaaacatttttgatctgctggaaaccaacaagactgctttaaatattacaaatttcttagtgagtcagaccactctggaagaggttttcatcaactttgccaaagaccagaagtcctatgaaactgctgataccagcagccaaggttccactataagtgttgactcacaagatgaccagatggagtcttaacacttccagcaaactcaatctcagcgtgtgaccaatggcttcattttgaagaaaagccacagaagatacacttccgcaagatatcttcattttaaagtaaagtaatatactgtatggaaagttacaactgtgttagactaacaagtaattataaaaggaaatttttccttctaaggtcagtgagtgttgttgctactgaaatgaattcctgtatactcaacactgtgagcatgctaatgtatatgctggtgattcttatgcaaaggtgaagccacctcaagatgaatatcttaatttattactttcaataaaaagacagtttaaaaggcatggattttggtagttgaaatataagagtggagaagaaaagtcagatggtttgtggcaggtgccaccgggcaagcagacaacataatttatttccagaaaacaacagaatgaacatcatcatgaatacatgaatcggctgtgatgtgtgaactgctaagggccaaatgaacgtttgnagagcagtgggcacaatgtttacaatgtatgngtatgtcactttcggtaccngtgaatgcatggggacgtgctgaacccgaaaaaaagtgcctttccataaggactgcaatagagagggcaatttaccctggtggtacacggaacctagattcactcctgccatnccttgccaatagtaagctgcagggtggaacaagaaatcacttgctctggggggaagggaggggggaatgggtgtgtcagctgggtagatacaaaccctgaaaagagaatccatgtgctnctggcaggcaacattttttaaagctctttcagaaaccctcatatttggggtttcttttcaggaaacattcctgtggagggaaaacgaatatgaagataattttcagctaattatctgggtgacccagaatcgtgtatatggctataggatagacttcttaataatggcaagtgacgtggccctggggaaaggtgctttatgtaccgtgtgtgcgtgtatgtgtgtgtatctatacaagtttgtcagctttggcatgactgtttgtctcgaaaaccaataaactcaaagtttagaaaaactcaaaaaaaaaaaaa

SEQ ID NO: 2 - DNA, 8875 bp, Homo sapiens
  n (1)..(8875): n = a, t, g, or c
gaagagttgattgagaagtgcctcttggttaaggattaaccacagggaaaaatccagcagaaacagaagaactgtgggtttcttaccccagccctcaaggaagctatgccgtgaaaggggtactgatacactgacatacagcaagttggacggggcatcagttcttcatttgtggagtggagaaaagaagaggaaatctctcatttggggcatttgaaggatggcttccctgtttcatcagcttcagatcctggtctggaaaaattggctaggtgtaaaaaggcagccgctttggacacttgtcttgatcttatggccagtcattattttcataattttggctattactcggaccaaatttcctccaactgcaaaaccaacttgttacctcgcacctcgaaaccttcctagtactggattctttccattcctgcagaccctactctgtgacacagactctaaatgcaaagacacaccctatggcccacaagatctgcttcgtaggaaaggaattgatgatgcactatttaaagacagtgagattctgagaaagtcatccaacctggataaggacagcagtttatcattccagagcacccaagttccagaaagaaggcatgcatcactagccacagtatttcccagtccaagttctgatttggaaatccccggaacatatactttcaatggcagtcaagtgctcgcacgaattcttggcttggaaaagctgttaaagcaaaattcaacttcagaagatatacgaagagaactatgtgacagctattcaggatacattgtggatgatgccttctcttggacctttctaggaagaaatgtttttaacaaattttgcctttctaacatgacccttttagagtcttctctccaagaactaaacaaacagttctcccagctatccagtgaccccaacaatcagaagatagtgtttcaggaaatagtcagaatgctgtctttcttctcacaagtgcaagagcagaaagctgtgtggcagcttctgtctagttttccaaatgtgtttcagaatgacacatcactaagcaatctatttgatgttcttcgaaaggcaaacagtgtgctgctggttgtgcagaaggtttatccacgttttgcaactaacgaaggtttcagaaccctccagaagtctgttaaacatctgctgtacactctggactccccagctcaaggtgactccgataatataacgcatgtgtggaatgaggatgatggacagaccttatctccaagcagtctggctgcacagctcctaattctggaaaactttgaagatgccctcttaaatatatcagcaaatagtccttatattccttacttggcatgtgtgagaaatgtgactgacagtttggccagaggttcaccagaaaatctaagactcctgcagtccacaatacgatttaaaaaatcttttcttcgcaatggttcctatgaagattactttcctccagttcctgaagtcctaaaatcaaaactgtctcaacttcgaaacttgaccgaacttctttgtgaatctgaaactttcagtttgatagagaagtcatgccagctctctgatatgagctttgggagcctgtgtgaagaaagtgagtttgatctgcaactcctcgaagcggcagagctgggcaccgaaatagcagccagcttactgtaccatgacaatgtcatatctaaaaaagtgagagatttgctgactggagatccaagcaaaattaatttaaatatggatcagtttctagaacaggcactgcaaatgaattacttggaaaatatcactcagttaataccgatcatagaagccatgctgcatgtcaataacagtgcagatgcttctgaaaagccaggtcagttactagaaatgtttaaaaatgttgaagagctgaaagaagatttaaggagaacaacaggaatgtccaacaggactattgacaagttgctggccattcccatccctgataatagagctgagattatttctcaggtgttctggctgcattcctgtgatactaatatcaccactcccaaactagaagatgcaatgaaagaattctgcaacctgtctctttcagagagatcccggcagtcttacctcatcggactcacccttctgcactacttaaacatttacaacttcacagacaaggtgtttttcccgaggaaagatcaaaagccagtagaaaagatgatggagctcttcataagactaaaagagattctcaatcagatggcttctggcacacatccgctgctagacaaaatgagatccctgaagcaaatgcatctgcccagaagtgttccattaacacaggcaatgtacagaagcaaccgaatgaacacaccacaaggatcatttagcaccatctcccaagcattatgttctcaaggaattaccactgaatatttaactgccatgctgccctcttcccagaggccaaaaggcaaccacaccaaggattttttgacttataaattaactaaagagcaaattgcttcaaaatatggaattcccataaataccacaccattttgcttctccctttataaagacatcattaacatgcccgctggacctgtgatttgggctttcttgaaacctatgttgttgggaagaattttgcatgcaccatataacccagtcacaaaggcaataatggaaaagtccaatgtaactctgagacagctggcggaattaagagaaaaatctcaagagtggatggataagtcgccacttttcatgaattccttccatctgttaaaccaggcaattccaatgctccagaatactctaaggaacccttttgtgcaagtttttgtaaagttctccgtgggactcgatgctgttgaactattgaaacagatagatgaactcgatattctaagactgaaattagagaacaacattgacatcatcgatcagcttaacacactatcttccctgacagtaaatatttcctcttgtgtattatatgaccgtattcaggcagcaaaaaccatagatgaaatggagagagaggctaaaaggctctacaaaagcaacgaactctttggaagtgttatttttaagcttccttctaacagaagctggcacagaggctatgactctggaaatgtctttcttcctcctgtcataaaatataccatccggatgagtctcaagaccgcacagaccacaagaagcctaagaaccaagatttgggctccagggccacacaattctccatcacacaaccagatctatggcagggcttttatttatttacaggatagtattgaaagagcaatcattgaattgcaaactggaaggaactcccaggaaatagcagtccaggttcaagcaattccttatccctgcttcatgaaagacaacttcctaaccagtgtctcttattctcttccaattgtgcttatggttgcctgggttgtatttatagctgcctttgtaaaaaagcttgtctatgagaaagacctccggcttcatgagtacatgaagatgatgggtgtgaactcctgcagccatttctttgcctggcttatagagagtgttggatttttactggttaccatcgtgatcctcatcattatactcaagtttggcaatattcttcctaaaacaaatgggttcattttgttcctgtatttttcggactacagcttctcggttattgccatgagctatcttatcagtgtcttcttcaacaacaccaacattgcagctctgatcggaagcctcatctacatcattgccttctttccatttattgttctggttacagtggagaatgagttgagctatgtattgaaagtgttcatgagcctgctgtccccaacagcattcagctatgcaagccaatacattgcacgatacgaagaacagggcattggtcttcagtgggaaaatatgtacacctccccggttcaggatgacaccacctcatttggctggctgtgctgtctaatcctagctgactctttcatttatttccttattgcttggtatgtcaggaatgtcttcccagggacatacggtatggcagctccctggtattttccaattcttccttcctattggaaggagcgatttgggtgtgcagaggtgaagcctgagaagagcaatggcctcatgtttactaacatcatgatgcagaacaccaacccatctgccagtcctgaatacatgttttcctctaacatcgagcctgaacctaaagatctcacagtcggggttgccctgcatggggtcacaaagatctatggctcaaaagttgctgttgataacctcaatctgaacttttatgaagggcatattacttcattgctggggcccaatggagctgggaaaactactaccatttccatgttaactgggctgtttggggcctcagcaggcaccatttttgtatatggaaaagatatcaaaacagacctacacacggtacggaagaacatgggagtctgtatgcagcacgacgtcttgttcagttacctcactactaaggagcaccttctcctatatggttccatcaaagttcctcactggactaaaaagcagctccacgaggaagtaaaaaggactttaaaagatactggactatatagccatcgtcataagagagttggaacactgtcaggaggcatgaagaggaagttatctatatccatagctctcattggtggatcaagggtagtaattttggatgaaccatctactggagttgacccatgttctcgccgaagtatatgggatgttatatccaagaacaaaactgccagaacaatcattctgtcaacgcaccacttggacgaggctgaagtgctgagtgaccgcatcgccttcctggagcagggtgggcttaggtgctgtgggtccccattttacctcaaggaagcctttggcgatgggtatcacctcacgcttaccaagaagaaggtctttctgaacttgaccaaagagtcacaaaaaaatagtgctatgagtcttgagcacttaacacaaaagaaaattgggaattccaatgccaatggcatctcaactcctgacgatttatctgtgagcagcagcaatttcacagacagagatgacaaaatcctgacaagaggagagaggctggatggctttggactgttgctgaagaagatcatggctatactcatcaagaggttccaccacgcccgcaggaactggaaaggtctcattgctcaggttatcctccccatcgtctttgttaccactgccatgggccttggcacactgagaaattccagcaacagttatccagagattcagatctccccctctctttatggtacctccgnacagacagccttctatgctaattatcacccgagcacggaagcacttgtctcagcaatgtgggacttccctggaattgacaacatgtgtctgaacaccagtgatctacagtgtttaaacaaagacagtctggaaaaatggaacaccagtggagaacccatcactaattttggtgtttgctcctgctcagaaaatgtccaggaatgtcctaaatttaactattccccaccgcacagaagaacttactcatcccaggtaatttataacctcactgggcaacgagtggaaaattatcttatatcaactgcaaatgagtttgtccaaaaaagatatggaggttggagttttgggctgcctttgacaaaagaccttcgttttgatataacaggagtccctgccaatagaacacttgccaaggtatggtatgatccagaaggctatcactcccttccagcttacctcaacagcctgaataatttccttctgcgagttaacatgtcaaaatacgatgctgcccgacatggcatcatcatgtatagccatccttatccaggagtgcaagaccaagaacaagccacaatcagcagtttaatcgatattttagtggcactgtctatcttgatgggctactctgtcaccaccgccagctttgtcacctatgttgtaagggaacatcaaaccaaagccaaacagttgcagcacatttcaggcattggcgtgacatgctactgggtaacaaacttcatttatgacatggttttctacttggtgcctgtagcgttttcaattggtatcattgcgattttcaaattacctgcattctacagtgaaaacaacctaggcgctgtatctctcctacttctcctgtttgggcatgcaacattttcctggatgtacttgctggctgggctcttccatgaaacaggaatggccttcatcacttacgtctgtgtcaacttgttttttggcattaattccattgtttccctgtcagtggtatactttctttccaaggaaaagcctaatgatccgactttagaacttatttctgaaaccctcaagcgcattttcctgattttcccacaattctgttttggctacggtttgattgaactttctcaacaacagtcggtcctagacttcttaaaagcatatggagtggaatacccaaatgaaacctttgagatgaataaactaggtgcaatgtttgtggctttggtttctcagggcaccatgtttttttccttgcgactcttaatcaacgaatccctgataaagaaactcaggcttttcttcagaaaatttaattcttcacatgtaagggagacaatagatgaggatgaagatgtgcgggctgagagattaagagttgagagtggtgcagctgaatttgacttggtccaactttattgtctcacaaagacctaccaacttatccacaaaaagattatagctgtaaacaacatcagcatcgggatacctgctggagagtgttttgggcttcttggagtgaatggagcaggaaagaccactatattcaagatgctgacaggagacatcattccttcaagtggaaacattctgatcagaaataagaccggatctctgggtcacgttgattctcacagctcattagttggctactgtcctcaggaagatgccttagatgacctggtaactgtggaagaacatttgtatttctatgccagggtacatggaattccagaaaaggatattaaagaaactgttcataaactccttaggagacttcacctgatgcccttcaaggacagagctacctctatgtgcagttatggcacaaaaagaaaattatccactgcactggccttgatagggaaaccttccattctactgctggatgagccgagctctggcatggatccgaagtcgaaacggcacctctggaagatcatttcagaagaagtacagaacaaatgttccgtcatcctcacatctcacagcatggaagaatgtgaagctctctgtaccaggttggccattatggtgaatggaaagtttcaatgtattggatctttgcagcacataaagagcaggtttggacgaggatttactgtcaaagttcacttgaagaataacaaagtgaccatggagaccctcacaaagttcatgcagctgcactttccaaaaacatacttaaaagatcagcacctcagcatgctagagtatcatgtaccagtcacagcaggaggagtcgcaaacatttttgatctgctggaaaccaacaagactgctttaaatattacaaatttcttagtgagtcagaccactctggaagaggttttcatcaactttgccaaagaccagaagtcctatgaaactgctgataccagcagccaaggttccactataagtgttgactcacaagatgaccagatggagtcttaacacttccagcaaactcaatctcagcgtgtgaccaatggcttcattttgaagaaaagccacagaagatacacttccgcaagatatcttcattttaaagtaaagtaatatactgtatggaaagttacaactgtgttagactaacaagtaattataaaaggaaatttttccttctaaggtcagtgagtgttgttgctactgaaatgaattcctgtatactcaacactgtgagcatgctaatgtatatgctggtgattcttatgcaaaggtgaagccacctcaagatgaatatcttaatttattactttcaataaaaagacagtttaaaaggcatggattttggtagttgaaatataagagtggagaagaaaagtcagatggtttgtggcaggtgccaccgggcaagcagacaacataatttatttccagaaaacaacagaatgaacatcatcatgaatacatgaatcggctgtgatgtgtgaactgctaagggccaaatgaacgtttgnagagcagtgggcacaatgtttacaatgtatgngtatgtcactttcggtaccngtgaatgcatggggacgtgctgaacccgaaaaaaagtgcctttccataaggactgcaatagagagggcaatttaccctggtggtacacggaacctagattcactcctgccatnccttgccaatagtaagctgcagggtggaacaagaaatcacttgctctggggggaagggaggggggaatgggtgtgtcagctgggtagatacaaaccctgaaaagagaatccatgtgctnctggcaggcaacattttttaaagctctttcagaaaccctcatatttggggtttcttttcaggaaacattcctgtggagggaaaacgaatatgaagataattttcagctaattatctgggtgacccagaatcgtgtatatggctataggatagacttcttaataatggcaagtgacgtggccctggggaaaggtgctttatgtaccgtgtgtgcgtgtatgtgtgtgtatctatacaagtttgtcagctttggcatgactgtttgtctcgaaaaccaataaactcaaagtttagaaaaactcaaaaaaaaaaaaa

SEQ ID NO: 3 - DNA, 8350 bp, Homo sapiens
gaagagttgattgagaagtgcctcttggttaaggattaaccacagggaaaaatccagcagaaacagaagaactgtgggtttcttaccccagccctcaaggaagctatgccgtgaaaggggtactgatacactgacatacagcaagttggacggggcatcagttcttcatttgtggagtggagaaaagaagaggaaatctctcatttggggcatttgaaggatggcttccctgtttcatcagcttcagatcctggtctggaaaaattggctaggtgtaaaaaggcagccgctttggacacttgtcttgatcttatggccagtcattattttcataattttggctattactcggaccaaatttcctccaactgcaaaaccaacttgttacctcgcacctcgaaaccttcctagtactggattctttccattcctgcagaccctactctgtgacacagactctaaatgcaaagacacaccctatggcccacaagatctgcttcgtaggaaaggaattgatgatgcactatttaaagacagtgagattctgagaaagtcatccaacctggataaggacagcagtttatcattccagagcacccaagttccagaaagaaggcatgcatcactagccacagtatttcccagtccaagttctgatttggaaatccccggaacatatactttcaatggcagtcaagtgctcgcacgaattcttggcttggaaaagctgttaaagcaaaattcaacttcagaagatatacgaagagaactatgtgacagctattcaggatacattgtggatgatgccttctcttggacctttctaggaagaaatgtttttaacaaattttgcctttctaacatgacccttttagagtcttctctccaagaactaaacaaacagttctcccagctatccagtgaccccaacaatcagaagatagtgtttcaggaaatagtcagaatgctgtctttcttctcacaagtgcaagagcagaaagctgtgtggcagcttctgtctagttttccaaatgtgtttcagaatgacacatcactaagcaatctatttgatgttcttcgaaaggcaaacagtgtgctgctggttgtgcagaaggtttatccacgttttgcaactaacgaaggtttcagaaccctccagaagtctgttaaacatctgctgtacactctggactccccagctcaaggtgactccgataatataacgcatgtgtggaatgaggatgatggacagaccttatctccaagcagtctggctgcacagctcctaattctggaaaactttgaagatgccctcttaaatatatcagcaaatagtccttatattccttacttggcatgtgtgagaaatgtgactgacagtttggccagaggttcaccagaaaatctaagactcctgcagtccacaatacgatttaaaaaatcttttcttcgcaatggttcctatgaagattactttcctccagttcctgaagtcctaaaatcaaaactgtctcaacttcgaaacttgaccgaacttctttgtgaatctgaaactttcagtttgatagagaagtcatgccagctctctgatatgagctttgggagcctgtgtgaagaaagtgagtttgatctgcaactcctcgaagcggcagagctgggcaccgaaatagcagccagcttactgtaccatgacaatgtcatatctaaaaaagtgagagatttgctgactggagatccaagcaaaattaatttaaatatggatcagtttctagaacaggcactgcaaatgaattacttggaaaatatcactcagttaataccgatcatagaagccatgctgcatgtcaataacagtgcagatgcttctgaaaagccaggtcagttactagaaatgtttaaaaatgttgaagagctgaaagaagatttaaggagaacaacaggaatgtccaacaggactattgacaagttgctggccattcccatccctgataatagagctgagattatttctcaggtgttctggctgcattcctgtgatactaatatcaccactcccaaactagaagatgcaatgaaagaattctgcaacctgtctctttcagagagatcccggcagtcttacctcatcggactcacccttctgcactacttaaacatttacaacttcacagacaaggtgtttttcccgaggaaagatcaaaagccagtagaaaagatgatggagctcttcataagactaaaagagattctcaatcagatggcttctggcacacatccgctgctagacaaaatgagatccctgaagcaaatgcatctgcccagaagtgttccattaacacaggcaatgtacagaagcaaccgaatgaacacaccacaaggatcatttagcaccatctcccaagcattatgttctcaaggaattaccactgaatatttaactgccatgctgccctcttcccagaggccaaaaggcaaccacaccaaggattttttgacttataaattaactaaagagcaaattgcttcaaaatatggaattcccataaataccacaccattttgcttctccctttataaagacatcattaacatgcccgctggacctgtgatttgggctttcttgaaacctatgttgttgggaagaattttgcatgcaccatataacccagtcacaaaggcaataatggaaaagtccaatgtaactctgagacagctggcggaattaagagaaaaatctcaagagtggatggataagtcgccacttttcatgaattccttccatctgttaaaccaggcaattccaatgctccagaatactctaaggaacccttttgtgcaagtttttgtaaagttctccgtgggactcgatgctgttgaactattgaaacagatagatgaactcgatattctaagactgaaattagagaacaacattgacatcatcgatcagcttaacacactatcttccctgacagtaaatatttcctcttgtgtattatatgaccgtattcaggcagcaaaaaccatagatgaaatggagagagaggctaaaaggctctacaaaagcaacgaactctttggaagtgttatttttaagcttccttctaacagaagctggcacagaggctatgactctggaaatgtctttcttcctcctgtcataaaatataccatccggatgagtctcaagaccgcacagaccacaagaagcctaagaaccaagatttgggctccagggccacacaattctccatcacacaaccagatctatggcagggcttttatttatttacaggatagtattgaaagagcaatcattgaattgcaaactggaaggaactcccaggaaatagcagtccaggttcaagcaattccttatccctgcttcatgaaagacaacttcctaaccagtgtctcttattctcttccaattgtgcttatggttgcctgggttgtatttatagctgcctttgtaaaaaagcttgtctatgagaaagacctccggcttcatgagtacatgaagatgatgggtgtgaactcctgcagccatttctttgcctggcttatagagagtgttggatttttactggttaccatcgtgatcctcatcattatactcaagtttggcaatattcttcctaaaacaaatgggttcattttgttcctgtatttttcggactacagcttctcggttattgccatgagctatcttatcagtgtcttcttcaacaacaccaacattgcagctctgatcggaagcctcatctacatcattgccttctttccatttattgttctggttacagtggagaatgagttgagctatgtattgaaagtgttcatgagcctgctgtccccaacagcattcagctatgcaagccaatacattgcacgatacgaagaacagggcattggtcttcagtgggaaaatatgtacacctccccggttcaggatgacaccacctcatttggctggctgtgctgtctaatcctagctgactctttcatttatttccttattgcttggtatgtcaggaatgtcttcccagggacatacggtatggcagctccctggtattttccaattcttccttcctattggaaggagcgatttgggtgtgcagaggtgaagcctgagaagagcaatggcctcatgtttactaacatcatgatgcagaacaccaacccatctgccagtcctgaatacatgttttcctctaacatcgagcctgaacctaaagatctcacagtcggggttgccctgcatggggtcacaaagatctatggctcaaaagttgctgttgataacctcaatctgaacttttatgaagggcatattacttcattgctggggcccaatggagctgggaaaactactaccatttccatgttaactgggctgtttggggcctcagcaggcaccatttttgtatatggaaaagatatcaaaacagacctacacacggtacggaagaacatgggagtctgtatgcagcacgacgtcttgttcagttacctcactactaaggagcaccttctcctatatggttccatcaaagttcctcactggactaaaaagcagctccacgaggaagtaaaaaggactttaaaagatactggactatatagccatcgtcataagagagttggaacactgtcaggaggcatgaagaggaagttatctatatccatagctctcattggtggatcaagggtagtaattttggatgaaccatctactggagttgacccatgttctcgccgaagtatatgggatgttatatccaagaacaaaactgccagaacaatcattctgtcaacgcaccacttggacgaggctgaagtgctgagtgaccgcatcgccttcctggagcagggtgggcttaggtgctgtgggtccccattttacctcaaggaagcctttggcgatgggtatcacctcacgcttaccaagaagaagagtccaaatttaaatgcaaatgcagtatgtgacaccatggccgtgacagcaatgatccaatcacatctccccgaagcctacctcaaggaggatattgggggagagcttgtttatgtacttcctccattcagcaccaaagtctcaggggcctacctgtcactcctacgggcactcgacaatggcatgggtgacctcaacatcgggtgctacggcatttcagataccaccgtggaggaggtctttctgaacttgaccaaagagtcacaaaaaaatagtgctatgagtcttgagcacttaacacaaaagaaaattgggaattccaatgccaatggcatctcaactcctgacgatttatctgtgagcagcagcaatttcacagacagagatgacaaaatcctgacaagaggagagaggctggatggctttggactgttgctgaagaagatcatggctatactcatcaagaggttccaccacrcccgcaggaactggaaaggtctcattgctcaggttatcctccccatcgtctttgttaccactgccatgggccttggcacactgagaaattccagcaacagttatccagagattcagatctccccctctctttatggtacctccgaacagacagccttctatgctaattatcacccgagcacggaagcacttgtctcagcaatgtgggacttccctggaattgacaacatgtgtctgaacaccagtgatctacagtgtttaaacaaagacagtctggaaaaatggaacaccagtggagaacccatcactaattttggtgtttgctcctgctcagaaaatgtccaggaatgtcctaaatttaactattccccaccgcacagaagaacttactcatcccaggtaatttataacctcactgggcaacgagtggaaaattatcttatatcaactgcaaatgagtttgtccaaaaaagatatggaggttggagttttgggctgcctttgacaaaagaccttcgttttgatataacaggagtccctgccaatagaacacttgccaaggtatggtatgatccagaaggctatcactcccttccagcttacctcaacagcctgaataatttccttctgcgagttaacatgtcaaaatacgatgctgcccgacatggcatcatcatgtatagccatccttatccaggagtgcaagaccaagaacaagccacaatcagcagtttaatcgatattttagtggcactgtctatcttgatgggctactctgtcaccaccgccagctttgtcacctatgttgtaagggaacatcaaaccaaagccaaacagttgcagcacatttcaggcattggcgtgacatgctactgggtaacaaacttcatttatgacatggttttctacttggtgcctgtagcgttttcaattggtatcattgcgattttcaaattacctgcattctacagtgaaaacaacctaggcgctgtatctctcctacttctcctgtttgggcatgcaacattttcctggatgtacttgctggctgggctcttccatgaaacaggaatggccttcatcacttacgtctgtgtcaacttgttttttggcattaattccattgtttccctgtcagtggtatactttctttccaaggaaaagcctaatgatccgactttagaacttatttctgaaaccctcaagcgcattttcctgattttcccacaattctgttttggctacggtttgattgaactttctcaacaacagtcggtcctagacttcttaaaagcatatggagtggaatacccaaatgaaacctttgagatgaataaactaggtgcaatgtttgtggctttggtttctcagggcaccatgtttttttccttgcgactcttaatcaacgaatccctgataaagaaactcaggcttttcttcagaaaatttaattcttcacatgtaagggagacaatagatgaggatgaagatgtgcgggctgagagattaagagttgagagtggtgcagctgaatttgacttggtccaactttattgtctcacaaagacctaccaacttatccacaaaaagattatagctgtaaacaacatcagcatcgggatacctgctggagagtgttttgggcttcttggagtgaatggagcaggaaagaccactatattcaagatgctgacaggagacatcattccttcaagtggaaacattctgatcagaaataagaccggatctctgggtcacgttgattctcacagctcattagttggctactgtcctcaggaagatgccttagatgacctggtaactgtggaagaacatttgtatttctatgccagggtacatggaattccagaaaaggatattaaagaaactgttcataaactccttaggagacttcacctgatgcccttcaaggacagagctacctctatgtgcagttatggcacaaaaagaaaattatccactgcactggccttgatagggaaaccttccattctactgctggatgagccgagctctggcatggatccgaagtcgaaacggcacctctggaagatcatttcagaagaagtacagaacaaatgttccgtcatcctcacatctcacagcatggaagaatgtgaagctctctgtaccaggttggccattatggtgaatggaaagtttcaatgtattggatctttgcagcacataaagagcaggtttggacgaggatttactgtcaaagttcacttgaagaataacaaagtgaccatggagaccctcacaaagttcatgcagctgcactttccaaaaacatacttaaaagatcagcacctcagcatgctagagtatcatgtaccagtcacagcaggaggagtcgcaaacatttttgatctgctggaaaccaacaagactgctttaaatattacaaatttcttagtgagtcagaccactctggaagaggttttcatcaactttgccaaagaccagaagtcctatgaaactgctgataccagcagccaaggttccactataagtgttgactcacaagatgaccagatggagtcttaacacttccagcaaactcaatctcagcgtgtgaccaatggcttcattttgaagaaaagccacagaagatacacttccgcaagatatcttcattttaaagtaaagtaatatactgtatggaaagttacaactgtgttagactaacaagtaattataaaaggaaatttttccttctaaggtcagtgagtgttgttgctactgaaatgaattcctgtatactcaacactgtgagcatgctaatgtatatgctggtgattcttatgcaaaggtgaagccacctcaagatgaatatcttaatttattactttcaataaaaagacagtttaaaaggcaaaaaaaaaaaaa

SEQ ID NO: 4 - DNA, 8113 bp, Homo sapiens
gaagagttgattgagaagtgcctcttggttaaggattaaccacagggaaaaatccagcagaaacagaagaactgtgggtttcttaccccagccctcaaggaagctatgccgtgaaaggggtactgatacactgacatacagcaagttggacggggcatcagttcttcatttgtggagtggagaaaagaagaggaaatctctcatttggggcatttgaaggatggcttccctgtttcatcagcttcagatcctggtctggaaaaattggctaggtgtaaaaaggcagccgctttggacacttgtcttgatcttatggccagtcattattttcataattttggctattactcggaccaaatttcctccaactgcaaaaccaacttgttacctcgcacctcgaaaccttcctagtactggattctttccattcctgcagaccctactctgtgacacagactctaaatgcaaagacacaccctatggcccacaagatctgcttcgtaggaaaggaattgatgatgcactatttaaagacagtgagattctgagaaagtcatccaacctggataaggacagcagtttatcattccagagcacccaagttccagaaagaaggcatgcatcactagccacagtatttcccagtccaagttctgatttggaaatccccggaacatatactttcaatggcagtcaagtgctcgcacgaattcttggcttggaaaagctgttaaagcaaaattcaacttcagaagatatacgaagagaactatgtgacagctattcaggatacattgtggatgatgccttctcttggacctttctaggaagaaatgtttttaacaaattttgcctttctaacatgacccttttagagtcttctctccaagaactaaacaaacagttctcccagctatccagtgaccccaacaatcagaagatagtgtttcaggaaatagtcagaatgctgtctttcttctcacaagtgcaagagcagaaagctgtgtggcagcttctgtctagttttccaaatgtgtttcagaatgacacatcactaagcaatctatttgatgttcttcgaaaggcaaacagtgtgctgctggttgtgcagaaggtttatccacgttttgcaactaacgaaggtttcagaaccctccagaagtctgttaaacatctgctgtacactctggactccccagctcaaggtgactccgataatataacgcatgtgtggaatgaggatgatggacagaccttatctccaagcagtctggctgcacagctcctaattctggaaaactttgaagatgccctcttaaatatatcagcaaatagtccttatattccttacttggcatgtgtgagaaatgtgactgacagtttggccagaggttcaccagaaaatctaagactcctgcagtccacaatacgatttaaaaaatcttttcttcgcaatggttcctatgaagattactttcctccagttcctgaagtcctaaaatcaaaactgtctcaacttcgaaacttgaccgaacttctttgtgaatctgaaactttcagtttgatagagaagtcatgccagctctctgatatgagctttgggagcctgtgtgaagaaagtgagtttgatctgcaactcctcgaagcggcagagctgggcaccgaaatagcagccagcttactgtaccatgacaatgtcatatctaaaaaagtgagagatttgctgactggagatccaagcaaaattaatttaaatatggatcagtttctagaacaggcactgcaaatgaattacttggaaaatatcactcagttaataccgatcatagaagccatgctgcatgtcaataacagtgcagatgcttctgaaaagccaggtcagttactagaaatgtttaaaaatgttgaagagctgaaagaagatttaaggagaacaacaggaatgtccaacaggactattgacaagttgctggccattcccatccctgataatagagctgagattatttctcaggtgttctggctgcattcctgtgatactaatatcaccactcccaaactagaagatgcaatgaaagaattctgcaacctgtctctttcagagagatcccggcagtcttacctcatcggactcacccttctgcactacttaaacatttacaacttcacagacaaggtgtttttcccgaggaaagatcaaaagccagtagaaaagatgatggagctcttcataagactaaaagagattctcaatcagatggcttctggcacacatccgctgctagacaaaatgagatccctgaagcaaatgcatctgcccagaagtgttccattaacacaggcaatgtacagaagcaaccgaatgaacacaccacaaggatcatttagcaccatctcccaagcattatgttctcaaggaattaccactgaatatttaactgccatgctgccctcttcccagaggccaaaaggcaaccacaccaaggattttttgacttataaattaactaaagagcaaattgcttcaaaatatggaattcccataaataccacaccattttgcttctccctttataaagacatcattaacatgcccgctggacctgtgatttgggctttcttgaaacctatgttgttgggaagaattttgcatgcaccatataacccagtcacaaaggcaataatggaaaagtccaatgtaactctgagacagctggcggaattaagagaaaaatctcaagagtggatggataagtcgccacttttcatgaattccttccatctgttaaaccaggcaattccaatgctccagaatactctaaggaacccttttgtgcaagtttttgtaaagttctccgtgggactcgatgctgttgaactattgaaacagatagatgaactcgatattctaagactgaaattagagaacaacattgacatcatcgatcagcttaacacactatcttccctgacagtaaatatttcctcttgtgtattatatgaccgtattcaggcagcaaaaaccatagatgaaatggagagagaggctaaaaggctctacaaaagcaacgaactctttggaagtgttatttttaagcttccttctaacagaagctggcacagaggctatgactctggaaatgtctttcttcctcctgtcataaaatataccatccggatgagtctcaagaccgcacagaccacaagaagcctaagaaccaagatttgggctccagggccacacaattctccatcacacaaccagatctatggcagggcttttatttatttacaggatagtattgaaagagcaatcattgaattgcaaactggaaggaactcccaggaaatagcagtccaggttcaagcaattccttatccctgcttcatgaaagacaacttcctaaccagtgtctcttattctcttccaattgtgcttatggttgcctgggttgtatttatagctgcctttgtaaaaaagcttgtctatgagaaagacctccggcttcatgagtacatgaagatgatgggtgtgaactcctgcagccatttctttgcctggcttatagagagtgttggatttttactggttaccatcgtgatcctcatcattatactcaagtttggcaatattcttcctaaaacaaatgggttcattttgttcctgtatttttcggactacagcttctcggttattgccatgagctatcttatcagtgtcttcttcaacaacaccaacattgcagctctgatcggaagcctcatctacatcattgccttctttccatttattgttctggttacagtggagaatgagttgagctatgtattgaaagtgttcatgagcctgctgtccccaacagcattcagctatgcaagccaatacattgcacgatacgaagaacagggcattggtcttcagtgggaaaatatgtacacctccccggttcaggatgacaccacctcatttggctggctgtgctgtctaatcctagctgactctttcatttatttccttattgcttggtatgtcaggaatgtcttcccagggacatacggtatggcagctccctggtattttccaattcttccttcctattggaaggagcgatttgggtgtgcagaggtgaagcctgagaagagcaatggcctcatgtttactaacatcatgatgcagaacaccaacccatctgccagtcctgaatacatgttttcctctaacatcgagcctgaacctaaagatctcacagtcggggttgccctgcatggggtcacaaagatctatggctcaaaagttgctgttgataacctcaatctgaacttttatgaagggcatattacttcattgctggggcccaatggagctgggaaaactactaccatttccatgttaactgggctgtttggggcctcagcaggcaccatttttgtatatggaaaagatatcaaaacagacctacacacggtacggaagaacatgggagtctgtatgcagcacgacgtcttgttcagttacctcactactaaggagcaccttctcctatatggttccatcaaagttcctcactggactaaaaagcagctccacgaggaagtaaaaaggactttaaaagatactggactatatagccatcgtcataagagagttggaacactgtcaggaggcatgaagaggaagttatctatatccatagctctcattggtggatcaagggtagtaattttggatgaaccatctactggagttgacccatgttctcgccgaagtatatgggatgttatatccaagaacaaaactgccagaacaatcattctgtcaacgcaccacttggacgaggctgaagtgctgagtgaccgcatcgccttcctggagcagggtgggcttaggtgctgtgggtccccattttacctcaaggaagcctttggcgatgggtatcacctcacgcttaccaagaagaaggtctttctgaacttgaccaaagagtcacaaaaaaatagtgctatgagtcttgagcacttaacacaaaagaaaattgggaattccaatgccaatggcatctcaactcctgacgatttatctgtgagcagcagcaatttcacagacagagatgacaaaatcctgacaagaggagagaggctggatggctttggactgttgctgaagaagatcatggctatactcatcaagaggttccaccacgcccgcaggaactggaaaggtctcattgctcaggttatcctccccatcgtctttgttaccactgccatgggccttggcacactgagaaattccagcaacagttatccagagattcagatctccccctctctttatggtacctccgracagacagccttctatgctaattatcacccgagcacggaagcacttgtctcagcaatgtgggacttccctggaattgacaacatgtgtctgaacaccagtgatctacagtgtttaaacaaagacagtctggaaaaatggaacaccagtggagaacccatcactaattttggtgtttgctcctgctcagaaaatgtccaggaatgtcctaaatttaactattccccaccgcacagaagaacttactcatcccaggtaatttataacctcactgggcaacgagtggaaaattatcttatatcaactgcaaatgagtttgtccaaaaaagatatggaggttggagttttgggctgcctttgacaaaagaccttcgttttgatataacaggagtccctgccaatagaacacttgccaaggtatggtatgatccagaaggctatcactcccttccagcttacctcaacagcctgaataatttccttctgcgagttaacatgtcaaaatacgatgctgcccgacatggcatcatcatgtatagccatccttatccaggagtgcaagaccaagaacaagccacaatcagcagtttaatcgatattttagtggcactgtctatcttgatgggctactctgtcaccaccgccagctttgtcacctatgttgtaagggaacatcaaaccaaagccaaacagttgcagcacatttcaggcattggcgtgacatgctactgggtaacaaacttcatttatgacatggttttctacttggtgcctgtagcgttttcaattggtatcattgcgattttcaaattacctgcattctacagtgaaaacaacctaggcgctgtatctctcctacttctcctgtttgggcatgcaacattttcctggatgtacttgctggctgggctcttccatgaaacaggaatggccttcatcacttacgtctgtgtcaacttgttttttggcattaattccattgtttccctgtcagtggtatactttctttccaaggaaaagcctaatgatccgactttagaacttatttctgaaaccctcaagcgcattttcctgattttcccacaattctgttttggctacggtttgattgaactttctcaacaacagtcggtcctagacttcttaaaagcatatggagtggaatacccaaatgaaacctttgagatgaataaactaggtgcaatgtttgtggctttggtttctcagggcaccatgtttttttccttgcgactcttaatcaacgaatccctgataaagaaactcaggcttttcttcagaaaatttaattcttcacatgtaagggagacaatagatgaggatgaagatgtgcgggctgagagattaagagttgagagtggtgcagctgaatttgacttggtccaactttattgtctcacaaagacctaccaacttatccacaaaaagattatagctgtaaacaacatcagcatcgggatacctgctggagagtgttttgggcttcttggagtgaatggagcaggaaagaccactatattcaagatgctgacaggagacatcattccttcaagtggaaacattctgatcagaaataagaccggatctctgggtcacgttgattctcacagctcattagttggctactgtcctcaggaagatgccttagatgacctggtaactgtggaagaacatttgtatttctatgccagggtacatggaattccagaaaaggatattaaagaaactgttcataaactccttaggagacttcacctgatgcccttcaaggacagagctacctctatgtgcagttatggcacaaaaagaaaattatccactgcactggccttgatagggaaaccttccattctactgctggatgagccgagctctggcatggatccgaagtcgaaacggcacctctggaagatcatttcagaagaagtacagaacaaatgttccgtcatcctcacatctcacagcatggaagaatgtgaagctctctgtaccaggttggccattatggtgaatggaaagtttcaatgtattggatctttgcagcacataaagagcaggtttggacgaggatttactgtcaaagttcacttgaagaataacaaagtgaccatggagaccctcacaaagttcatgcagctgcactttccaaaaacatacttaaaagatcagcacctcagcatgctagagtatcatgtaccagtcacagcaggaggagtcgcaaacatttttgatctgctggaaaccaacaagactgctttaaatattacaaatttcttagtgagtcagaccactctggaagaggttttcatcaactttgccaaagaccagaagtcctatgaaactgctgataccagcagccaaggttccactataagtgttgactcacaagatgaccagatggagtcttaacacttccagcaaactcaatctcagcgtgtgaccaatggcttcattttgaagaaaagccacagaagatacacttccgcaagatatcttcattttaaagtaaagtaatatactgtatggaaagttacaactgtgttagactaacaagtaattataaaaggaaatttttccttctaaggtcagtgagtgttgttgctactgaaatgaattcctgtatactcaacactgtgagcatgctaatgtatatgctggtgattcttatgcaaaggtgaagccacctcaagatgaatatcttaatttattactttcaataaaaagacagtttaaaaggcaaaaaaaaaaaaa

SEQ ID NO: 5 - protein, 2595 aa, Homo sapiens
  Xaa (1)..(2595): Xaa = any amino acid
MASLFHQLQILVWKNWLGVKRQPLWTLVLILWPVIIFIILAITRTKFPPTAKPTCYLAPRNLPSTGFFPFLQTLLCDTDSKCKDTPYGPQDLLRRKGIDDALFKDSEILRKSSNLDKDSSLSFQSTQVPERRHASLATVFPSPSSDLEIPGTYTFNGSQVLARILGLEKLLKQNSTSEDIRRELCDSYSGYIVDDAFSWTFLGRNVFNKFCLSNMTLLESSLQELNKQFSQLSSDPNNQKIVFQEIVRMLSFFSQVQEQKAVWQLLSSFPNVFQNDTSLSNLFDVLRKANSVLLVVQKVYPRFATNEGFRTLQKSVKHLLYTLDSPAQGDSDNITHVWNEDDGQTLSPSSLAAQLLILENFEDALLNISANSPYIPYLACVRNVTDSLARGSPENLRLLQSTIRFKKSFLRNGSYEDYFPPVPEVLKSKLSQLRNLTELLCESETFSLIEKSCQLSDMSFGSLCEESEFDLQLLEAAELGTEIAASLLYHDNVISKKVRDLLTGDPSKINLNMDQFLEQALQMNYLENITQLIPIIEAMLHVNNSADASEKPGQLLEMFKNVEELKEDLRRTTGMSNRTIDKLLAIPIPDNRAEIISQVFWLHSCDTNITTPKLEDAMKEFCNLSLSERSRQSYLIGLTLLHYLNIYNFTDKVFFPRKDQKPVEKMMELFIRLKEILNQMASGTHPLLDKMRSLKQMHLPRSVPLTQAMYRSNRMNTPQGSFSTISQALCSQGITTEYLTAMLPSSQRPKGNHTKDFLTYKLTKEQIASKYGIPINTTPFCFSLYKDIINMPAGPVIWAFLKPMLLGRILHAPYNPVTKAIMEKSNVTLRQLAELREKSQEWMDKSPLFMNSFHLLNQAIPMLQNTLRNPFVQVFVKFSVGLDAVELLKQIDELDILRLKLENNIDIIDQLNTLSSLTVNISSCVLYDRIQAAKTIDEMEREAKRLYKSNELFGSVIFKLPSNRSWHRGYDSGNVFLPPVIKYTIRMSLKTAQTTRSLRTKIWAPGPHNSPSHNQIYGRAFIYLQDSIERAIIELQTGRNSQEIAVQVQAIPYPCFMKDNFLTSVSYSLPIVLMVAWVVFIAAFVKKLVYEKDLRLHEYMKMMGVNSCSHFFAWLIESVGFLLVTIVILIIILKFGNILPKTNGFILFLYFSDYSFSVIAMSYLISVFFNNTNIAALIGSLIYIIAFFPFIVLVTVENELSYVLKVFMSLLSPTAFSYASQYIARYEEQGIGLQWENMYTSPVQDDTTSFGWLCCLILADSFIYFLIAWYVRNVFPGTYGMAAPWYFPILPSYWKERFGCAEVKPEKSNGLMFTNIMMQNTNPSASPEYMFSSNIEPEPKDLTVGVALHGVTKIYGSKVAVDNLNLNFYEGHITSLLGPNGAGKTTTISMLTGLFGASAGTIFVYGKDIKTDLHTVRKNMGVCMQHDVLFSYLTTKEHLLLYGSIKVPHWTKKQLHEEVKRTLKDTGLYSHRHKRVGTLSGGMKRKLSISIALIGGSRVVILDEPSTGVDPCSRRSIWDVISKNKTARTIILSTHHLDEAEVLSDRIAFLEQGGLRCCGSPFYLKEAFGDGYHLTLTKKKSPNLNANAVCDTMAVTAMIQSHLPEAYLKEDIGGELVYVLPPFSTKVSGAYLSLLRALDNGMGDLNIGCYGISDTTVEEVFLNLTKESQKNSAMSLEHLTQKKIGNSNANGISTPDDLSVSSSNFTDRDDKILTRGERLDGFGLLLKKIMAILIKRFHHXRRNWKGLIAQVILPIVFVTTAMGLGTLRNSSNSYPEIQISPSLYGTSEQTAFYANYHPSTEALVSAMWDFPGIDNMCLNTSDLQCLNKDSLEKWNTSGEPITNFGVCSCSENVQECPKFNYSPPHRRTYSSQVIYNLTGQRVENYLISTANEFVQKRYGGWSFGLPLTKDLRFDITGVPANRTLAKVWYDPEGYHSLPAYLNSLNNFLLRVNMSKYDAARHGIIMYSHPYPGVQDQEQATISSLIDILVALSILMGYSVTTASFVTYVVREHQTKAKQLQHISGIGVTCYWVTNFIYDMVFYLVPVAFSIGIIAIFKLPAFYSENNLGAVSLLLLLFGHATFSWMYLLAGLFHETGMAFITYVCVNLFFGINSIVSLSVVYFLSKEKPNDPTLELISETLKRIFLIFPQFCFGYGLIELSQQQSVLDFLKAYGVEYPNETFEMNKLGAMFVALVSQGTMFFSLRLLINESLIKKLRLFFRKFNSSHVRETIDEDEDVRAERLRVESGAAEFDLVQLYCLTKTYQLIHKKIIAVNNISIGIPAGECFGLLGVNGAGKTTIFKMLTGDIIPSSGNILIRNKTGSLGHVDSHSSLVGYCPQEDALDDLVTVEEHLYFYARVHGIPEKDIKETVHKLLRRLHLMPFKDRATSMCSYGTKRKLSTALALIGKPSILLLDEPSSGMDPKSKRHLWKIISEEVQNKCSVILTSHSMEECEALCTRLAIMVNGKFQCIGSLQHIKSRFGRGFTVKVHLKNNKVTMETLTKFMQLHFPKTYLKDQHLSMLEYHVPVTAGGVANIFDLLETNKTALNITNFLVSQTTLEEVFINFAKDQKSYETADTSSQGSTISVDSQDDQMES

SEQ ID NO: 6 - protein, 2516 aa, Homo sapiens
  Xaa (1)..(2516): Xaa = any amino acid
MASLFHQLQILVWKNWLGVKRQPLWTLVLILWPVIIFIILAITRTKFPPTAKPTCYLAPRNLPSTGFFPFLQTLLCDTDSKCKDTPYGPQDLLRRKGIDDALFKDSEILRKSSNLDKDSSLSFQSTQVPERRHASLATVFPSPSSDLEIPGTYTFNGSQVLARILGLEKLLKQNSTSEDIRRELCDSYSGYIVDDAFSWTFLGRNVFNKFCLSNMTLLESSLQELNKQFSQLSSDPNNQKIVFQEIVRMLSFFSQVQEQKAVWQLLSSFPNVFQNDTSLSNLFDVLRKANSVLLVVQKVYPRFATNEGFRTLQKSVKHLLYTLDSPAQGDSDNITHVWNEDDGQTLSPSSLAAQLLILENFEDALLNISANSPYIPYLACVRNVTDSLARGSPENLRLLQSTIRFKKSFLRNGSYEDYFPPVPEVLKSKLSQLRNLTELLCESETFSLIEKSCQLSDMSFGSLCEESEFDLQLLEAAELGTEIAASLLYHDNVISKKVRDLLTGDPSKINLNMDQFLEQALQMNYLENITQLIPIIEAMLHVNNSADASEKPGQLLEMFKNVEELKEDLRRTTGMSNRTIDKLLAIPIPDNRAEIISQVFWLHSCDTNITTPKLEDAMKEFCNLSLSERSRQSYLIGLTLLHYLNIYNFTDKVFFPRKDQKPVEKMMELFIRLKEILNQMASGTHPLLDKMRSLKQMHLPRSVPLTQAMYRSNRMNTPQGSFSTISQALCSQGITTEYLTAMLPSSQRPKGNHTKDFLTYKLTKEQIASKYGIPINTTPFCFSLYKDIINMPAGPVIWAFLKPMLLGRILHAPYNPVTKAIMEKSNVTLRQLAELREKSQEWMDKSPLFMNSFHLLNQAIPMLQNTLRNPFVQVFVKFSVGLDAVELLKQIDELDILRLKLENNIDIIDQLNTLSSLTVNISSCVLYDRIQAAKTIDEMEREAKRLYKSNELFGSVIFKLPSNRSWHRGYDSGNVFLPPVIKYTIRMSLKTAQTTRSLRTKIWAPGPHNSPSHNQIYGRAFIYLQDSIERAIIELQTGRNSQEIAVQVQAIPYPCFMKDNFLTSVSYSLPIVLMVAWVVFIAAFVKKLVYEKDLRLHEYMKMMGVNSCSHFFAWLIESVGFLLVTIVILIIILKFGNILPKTNGFILFLYFSDYSFSVIAMSYLISVFFNNTNIAALIGSLIYIIAFFPFIVLVTVENELSYVLKVFMSLLSPTAFSYASQYIARYEEQGIGLQWENMYTSPVQDDTTSFGWLCCLILADSFIYFLIAWYVRNVFPGTYGMAAPWYFPILPSYWKERFGCAEVKPEKSNGLMFTNIMMQNTNPSASPEYMFSSNIEPEPKDLTVGVALHGVTKIYGSKVAVDNLNLNFYEGHITSLLGPNGAGKTTTISMLTGLFGASAGTIFVYGKDIKTDLHTVRKNMGVCMQHDVLFSYLTTKEHLLLYGSIKVPHWTKKQLHEEVKRTLKDTGLYSHRHKRVGTLSGGMKRKLSISIALIGGSRVVILDEPSTGVDPCSRRSIWDVISKNKTARTIILSTHHLDEAEVLSDRIAFLEQGGLRCCGSPFYLKEAFGDGYHLTLTKKKVFLNLTKESQKNSAMSLEHLTQKKIGNSNANGISTPDDLSVSSSNFTDRDDKILTRGERLDGFGLLLKKIMAILIKRFHHARRNWKGLIAQVILPIVFVTTAMGLGTLRNSSNSYPEIQISPSLYGTSXQTAFYANYHPSTEALVSAMWDFPGIDNMCLNTSDLQCLNKDSLEKWNTSGEPITNFGVCSCSENVQECPKFNYSPPHRRTYSSQVIYNLTGQRVENYLISTANEFVQKRYGGWSFGLPLTKDLRFDITGVPANRTLAKVWYDPEGYHSLPAYLNSLNNFLLRVNMSKYDAARHGIIMYSHPYPGVQDQEQATISSLIDILVALSILMGYSVTTASFVTYVVREHQTKAKQLQHISGIGVTCYWVTNFIYDMVFYLVPVAFSIGIIAIFKLPAFYSENNLGAVSLLLLLFGHATFSWMYLLAGLFHETGMAFITYVCVNLFFGINSIVSLSVVYFLSKEKPNDPTLELISETLKRIFLIFPQFCFGYGLIELSQQQSVLDFLKAYGVEYPNETFEMNKLGAMFVALVSQGTMFFSLRLLINESLIKKLRLFFRKFNSSHVRETIDEDEDVRAERLRVESGAAEFDLVQLYCLTKTYQLIHKKIIAVNNISIGIPAGECFGLLGVNGAGKTTIFKMLTGDIIPSSGNILIRNKTGSLGHVDSHSSLVGYCPQEDALDDLVTVEEHLYFYARVHGIPEKDIKETVHKLLRRLHLMPFKDRATSMCSYGTKRKLSTALALIGKPSILLLDEPSSGMDPKSKRHLWKIISEEVQNKCSVILTSHSMEECEALCTRLAIMVNGKFQCIGSLQHIKSRFGRGFTVKVHLKNNKVTMETLTKFMQLHFPKTYLKDQHLSMLEYHVPVTAGGVANIFDLLETNKTALNITNFLVSQTTLEEVFINFAKDQKSYETADTSSQGSTISVDSQDDQMES

SEQ ID NO: 7 - DNA, 21 bp, Artificial Sequence
  primer
gaagagttgattgagaagtgc

SEQ ID NO: 8 - DNA, 21 bp, Artificial Sequence
  primer
cgaagagaactatgtgacagc

SEQ ID NO: 9 - DNA, 20 bp, Artificial Sequence
  primer
cttctcacaagtgcaagagc

SEQ ID NO: 10 - DNA, 24 bp, Artificial Sequence
  primer
cgcaatggttcctatgaagattac

SEQ ID NO: 11 - DNA, 28 bp, Artificial Sequence
  primer
cagaagggtgagtccgatgaggtaagac

SEQ ID NO: 12 - DNA, 21 bp, Artificial Sequence
  primer
gctgtcacatagttctcttcg

SEQ ID NO: 13 - DNA, 24 bp, Artificial Sequence
  primer
gtaatcttcataggaaccattgcg

SEQ ID NO: 14 - DNA, 25 bp, Artificial Sequence
  primer
cctacacacggtacggaagaacatg

SEQ ID NO: 15 - DNA, 27 bp, Artificial Sequence
  primer
gccatcgtcataagagagttggaacac

SEQ ID NO: 16 - DNA, 19 bp, Artificial Sequence
  primer
gtgcttatggttgcctggg

SEQ ID NO: 17 - DNA, 20 bp, Artificial Sequence
  primer
cttccatctgttaaaccagg

SEQ ID NO: 18 - DNA, 18 bp, Artificial Sequence
  primer
ggtgttctggctgcattc

SEQ ID NO: 19 - DNA, 20 bp, Artificial Sequence
  primer
gcctcatctacatcattgcc

SEQ ID NO: 20 - DNA, 27 bp, Artificial Sequence
  primer
gtgttccaactctcttatgacgatggc

SEQ ID NO: 21 - DNA, 25 bp, Artificial Sequence
  primer
catgttcttccgtaccgtgtgtagg

SEQ ID NO: 22 - DNA, 20 bp, Artificial Sequence
  primer
ggcaatgatgtagatgaggc

SEQ ID NO: 23 - DNA, 19 bp, Artificial Sequence
  primer
cccaggcaaccataagcac

SEQ ID NO: 24 - DNA, 30 bp, Artificial Sequence
  primer
cttttctactggcttttgatctttcctcgg

SEQ ID NO: 25 - DNA, 19 bp, Artificial Sequence
  primer
ccttgatagggaaaccttc

SEQ ID NO: 26 - DNA, 20 bp, Artificial Sequence
  primer
caccagcatatacattagca

SEQ ID NO: 27 - DNA, 19 bp, Artificial Sequence
  primer
gaaggtttccctatcaagg

SEQ ID NO: 28 - DNA, 27 bp, Artificial Sequence
  primer
gtatcatgtaccagtcacagcaggagg

SEQ ID NO: 29 - DNA, 28 bp, Artificial Sequence
  primer
ccaaagaccagaagtcctatgaaactgc

SEQ ID NO: 30 - DNA, 20 bp, Artificial Sequence
  primer
gagtggagaagaaaagtcag

SEQ ID NO: 31 - DNA, 21 bp, Artificial Sequence
  primer
cacggaacctagattcactcc

SEQ ID NO: 32 - DNA, 18 bp, Artificial Sequence
  primer
cccagagcaagtgatttc

SEQ ID NO: 33 - DNA, 18 bp, Artificial Sequence
  primer
cgagtgcccgtaggagtg

SEQ ID NO: 34 - DNA, 22 bp, Artificial Sequence
  primer
ttgcacctagtttattcatctc

SEQ ID NO: 35 - DNA, 23 bp, Artificial Sequence
  primer
gtcataaatgaagtttgttaccc

SEQ ID NO: 36 - DNA, 21 bp, Artificial Sequence
  primer
caacagttatccagagattca

SEQ ID NO: 37 - DNA, 19 bp, Artificial Sequence
  primer
gagtccctgccaatagaac

SEQ ID NO: 38 - DNA, 20 bp, Artificial Sequence
  primer
gcaaatgcagtatgtgacac